(19) United States
(12) Patent Application Publication
(10) Pub. No.: US 2008/0171916 A1
(43) Pub. Date: Jul. 17, 2008

(54) Practical computer program that diagnoses diseases in actual patients

(76) Inventors: Carlos Feder, Palo Alto, CA (US); Tomas Feder, Palo Alto, CA (US)

Correspondence Address: CARLOS FEDER, 433 TENNESSEE LANE, PALO ALTO, CA, 94306, US

(21) Appl. No.: 12/075,609

(22) Filed: Mar. 13, 2008

Related U.S. Application Data
(63) Continuation-in-part of application No. 11/133,726, filed on May 20, 2005.

(51) Int. Cl. A61B 5/00

(52) U.S. Cl. 600/300

(57) ABSTRACT

This algorithm and corresponding computer program emulates the diagnostic reasoning of a clinician. Accurate and efficient, it concludes only those final diagnoses that agree with the diseases that actually afflict a patient. A differential diagnosis list is created and the probability of each diagnosis is calculated with a novel procedure that we call Mini-Max Procedure that uses the positive predictive value of clinical data present to increase probability and the sensitivity of clinical data absent to reduce probability. The probability of a diagnosis is considered equal to the maximum positive predictive value of all clinical data present that support the diagnosis, circumventing more complex and inaccurate prior art methods. The Mini-Max Procedure also identifies concurrent diseases. Bayes formula, because of its inability to process properly interdependent clinical data and concurrent diseases, is used with modifications. The algorithm recommends at each diagnostic step, the best cost-benefit clinical datum next to investigate. Furthermore, the algorithm can simultaneously recommend several best cost-benefit clinical data, avoiding the need to contact the patient after the result of each single test is obtained. Heuristic parameters and abridged output files reduce the great number of best cost benefit clinical data recommended, without compromising the accuracy of the diagnostic procedure. Interactions of drugs and concurrent diseases with clinical data of the primary disease is detected, precluding ruling out of serious diseases due to this masking effect. Overlooking of important diagnoses is precluded by searching and processing diagnoses that are related to confirmed diagnoses. The algorithm diagnoses clinical forms of disease and complex clinical presentations, where disease, syndromes, complications, and other clinical entities coexist in a single patient. The algorithm processes efficiently synonyms of clinical data and diagnoses. The algorithm is straightforward, logical and mathematically simple; heuristic restrictions preclude excessive proliferation of clinical data and diagnoses. Because it is expressed in natural language, it is readily understandable and user friendly.

### CROSS-REFERENCE TO RELATED APPLICATIONS

This application is a continuation-in-part of application Ser. No. 11/133,726 filed on May 20, 2005, titled COMPUTERIZED MEDICAL DIAGNOSIS: A FRESH APPROACH TO AN OLD UNSOLVED PROBLEM.

### FEDERALLY SPONSORED RESEARCH

Not Applicable SEQUENCE LISTING OR PROGRAM

Not Applicable

### BACKGROUND OF THE INVENTION

1. Field of Invention

Our invention is a novel computer algorithm that diagnoses diseases in actual patients; it encompasses the fields of medicine and computer science.

2. Prior Art

Existing diagnostic programs, some commercially available, typically offer only limited diagnostic information and are considered clinical training tools rather than an aid to diagnose diseases afflicting actual patients. Our computer system offers the following novel advantages:

It confirms one or more diseases that indeed afflict a specific patient, instead of a long list of potential diagnoses that compete with each other.

It is programmed in a way that enables processing any known disease, including very rare diseases that other programs exclude.

It diagnoses concurrent diseases afflicting simultaneously a specific patient.

It calculates accurately the probability of each diagnosis with our novel mini-max procedure, in contrast with some other programs that rely on Bayes formula, inadequate for this purpose, because this formula requires that clinical data (symptom, physical sign, test or procedure results) manifested by a patient be independent and exhaustive, and diagnoses be incompatible, conditions that are not fulfilled by internal medicine and actual clinical cases. Bayes calculation deals only with competing diagnoses and is unable to diagnose concurrent diseases.

It gives primary importance to the cost of obtaining each clinical datum. Our algorithm interprets cost not only as dollar expense, but also includes discomfort and risk of the procedure; it uses a novel method of qualitatively estimating these three elements and considering the greatest of such elements as equal to the overall cost. It makes little sense to order expensive, uncomfortable, and risky tests or procedures, before resorting to less costly ones that may suffice to confirm or rule out a diagnosis.

It recommends the probabilistically calculated best cost-benefit clinical data to investigate next in the patient at each diagnostic step, to achieve more efficiently and economically, a final diagnosis. This best choice function eliminates the request of many unnecessary tests, which is extremely important in this era of managed care, in which medical insurers are pressuring physicians to reduce expenses; on the other hand it will preclude curtailing really necessary tests or procedures. It also would protect physicians from unjustified malpractice suits; if the computer program is flawless and universally respected, its recommendations will become standard of care.

It recommends a set of best cost-benefit clinical data to be investigated simultaneously in the patient, based on diverse heuristic strategies. This is essential in emergency situations, but also important in an outpatient setting, to avoid the need of the physician to contact the patient after each single new test result to order the next one.

It can display, for clinical analysis purposes, the entire set of recommended best cost-benefit clinical data able to impact the probability of potential diagnoses; on the other hand, as the number of such data may be huge, it can display selected partial lists that turns the diagnostic task more economic and manageable, without compromising the accuracy of the result. It accomplishes this through parameters that can be set at diverse values and through abridged output files, giving the user the choice to select the ones that fit best his or her preferences.

It includes complex clinical presentations models in the database, which list associations among diseases, such as causes, complications, and other relations; each confirmed diagnosis is compared to these models and when a match is found, all the other diagnoses in the model are processed for presence of absence in the patient. This precludes overlooking associated diseases.

It deals with interactions between concurrent diseases or drugs that mask important clinical data of the primary disease, precluding the improper ruling out of the corresponding diagnosis.

It is ease to update, simply by adding to the database a newly developed clinical datum (test or procedure) for a known disease or a newly discovered disease with the corresponding clinical data and their sensitivities (frequency with which the disease manifest each clinical datum). Finally, including the disease in a complex clinical presentation model, if some associations exist with other diseases or having masking properties.

Such an algorithm, if successful in medicine, may represent a more general model of reasoning; a paradigm of mental structure and functioning applicable to other inexact disciplines such as law, sociology, politics, defense, or corporate strategy.

Most prior diagnostic programs are based on entangled networks, Bayesian networks, neural networks, any of them difficult or impossible to implement and update for a program like ours, devised to encompass all known diseases. Our algorithm is straightforward, involving only simple mathematical formulas, limited probability calculations, and categorical matching of patient clinical data and diagnoses with diverse kind of models (disease models, clinical entity models, and complex clinical presentation models). In the following paragraphs we explain the basic principles of our algorithm.

### Mini-Max Procedure

The Mini-Max Procedure enables the simultaneous mathematical processing of clinical data present in a patient, that favor a diagnosis and the clinical data absent that disfavor this diagnosis, in the same patient. The resulting probability of this diagnosis, based on these clinical data, is more accurate than other methods because it circumvents improper use of Bayes formula. The Mini-Max Procedure has important properties, which will be described in detail in this application. The Mini-Max Procedure will be described functioning with medical diagnosis as a model or paradigm of human reasoning; but we also expect it to be applicable to other inexact disciplines, such as finance, law, defense, sociology, politics, and others. If possible, we would like the scope of this patent extended to applications other than medical diagnosis.

### Best Cost-Benefit Clinical Datum to Investigate Next in a Patient

Best cost-benefit clinical datum to investigate next in the patient is an important function that can substantially shorten and reduce the cost of a diagnostic quest by precluding a search for futile clinical data. Prior art recommends clinical data to be investigated in a patient, but our algorithm resolves this function in a more accurate and novel manner, based on our original Mini-Max Procedure.

### Simultaneous Recommendation of Several Best Cost-Benefit Clinical Data Next to Investigate

Serially recommending single best cost-benefit clinical data is impractical; were the patient to require several tests, she/he would need to be contacted repeatedly for additional instructions. For this reason, physicians often simultaneously order a set of several analyses, tests, or procedures; this approach is critical for emergency cases. Our algorithm can emulate such human behavior by iterating the best cost-benefit clinical data function, first assuming each newly recommended best cost-benefit clinical datum as virtually present and then as virtually absent, while observing the effect that each iteration has on the probability of each diagnosis. After several iterations, a set of several best cost-benefit clinical data to be investigated in the patient is recommended.

### Parameters and Abridged Output Files to Reduce the Number, Cost, and Burden to Manage a Huge Number of Recommended Cost-Benefit Clinical Data

When several diagnoses are processed, the number of recommended cost-benefit clinical data to investigate can be quite numerous. Our algorithm is able to reduce this number without compromising the accuracy of the diagnostic process. This was achieved by creating Parameters that can be set at empirical values and Abridged output files recommending a reduced number of such clinical data. These functions will be explained in the DESCRIPTION and FLOW CHART.

The Greatest Positive Predictive Value of Clinical Data Supporting a Diagnosis Equals the Probability of this Diagnosis

Authors use values such as sensitivity, specificity, predictive value, weights, or an estimated value of clinical data to indicate how strongly these clinical data support a corresponding diagnosis. These authors add, subtract, multiply, or average these values supporting a diagnosis, or iterate Bayes formula with each additional clinical datum to establish a total score or probability of the diagnosis. Sensitivity (S) of a clinical datum for a specific disease (frequency with which the disease manifest such clinical datum) can be established statistically. Positive predictive value (PP value) of a clinical datum for a specific disease, that we consider the most reliable index of how strongly this clinical datum supports the corresponding disease, is derived in our diagnostic program from S values. This is achieved by an equation that yields mathematically accurate PP values that can be calculated before the program is delivered to the user, as opposed to be calculated in real time, which saves computational time.

Because many clinical data that a patient manifest are intimately related by a common cause or lesion, they have many times a similar meaning. For example, jaundice, dark urine, and increased direct serum bilirubin are clinical data related by similar pathophysiologic mechanisms generated by a single lesion: biliary tract obstruction. Were we arithmetically to combine the several PP values of these three equivalent and “redundant” clinical data, the probability (P) of diagnosis biliary tract obstruction would be improperly increased thereby providing an undue advantage to this diagnosis, as compared to competing diagnoses. Additionally, were common bile duct-obstructing gallstones revealed by endoscopic retrograde cholangiopancreatography (ERCP)—a clinical datum that alone has a PP value of 1—the P of obstructing gallstones as a diagnosis, if supporting clinical data values are added, would exceed 1, which is probabilistically impossible. With our algorithm, the PP value of gallstone obstruction supersedes all other clinical data with lesser PP value (jaundice, dark urine, increased serum bilirubin) because—whether present or absent—they would not change the diagnosis of obstructing gallstones already confirmed by ERCP. We equal the greatest positive predictive value of all clinical data present that support a diagnosis to the probability of this diagnosis. Considering the maximum positive predictive value of clinical data supporting a specific diagnosis equal to the probability of this diagnosis is more rational, more accurate, and more efficient than the other mentioned probabilistic or scoring methods.

### Competing Diagnosis and Concurrent Diseases

Distinguishing competing diagnoses from concurrent diseases is a fundamental property of the Mini-Max Procedure.

### Disregarding Qualities of Clinical Data

Clinical data, especially subjective symptoms, typically have diverse non-exclusive qualities. For example, angina pectoris typically is retrosternal, radiating to the neck, jaw, and upper extremities; is oppressive, lasting only a few minutes; is exertion related and relieved by nitroglycerine. Some authors confer values to these qualities, their chronology, and their evolution. This is correct, when such qualities, powerfully suggest a diagnosis. Nevertheless, our algorithm purposely does not consider such clinical data qualities; we believe that computation of clinical datum qualities is not critical for calculation of probability (P) of a diagnosis. Reasons will be discussed in the DESCRIPTION

### Disregarding Prevalence of Diseases

Purposely disregarding disease prevalence is doubly advantageous: (1) Prior probabilities of diseases (equivalent to prevalence) are eliminated from Bayes formula, which is transformed into a simplified equation for calculating the PP value from statistically established S values of clinical data. (2) Small prevalence no longer is a reason for excluding a rare disease from a differential diagnosis list; this disease must get a chance to become a final diagnosis based solely on merit of its supporting clinical data.

### Complex Clinical Presentations

Complex clinical presentations, quite frequent in actual cases, are combinations of related or unrelated concurrent diseases in a single patient. Our algorithm partitions the entire diagnostic process into two steps: (1) uses probability to confirm or rule out single diagnoses; (2) identifies and integrates categorically, concurrent diseases into complex clinical presentations. These kinds of presentations cannot be processed probabilistically because of the computational complexity involved; they must be linked categorically.

### Complex Clinical Presentations Models

Complex clinical presentation models are files that list single diagnoses that are related in some pathophysiologic or statistical way. They have three functions in our algorithm: (1) precluding overlooking related concurrent diagnoses; once a confirmed diagnosis matches any of the diagnoses in the model, all the other diagnoses are processed for presence or absence; (2) detecting interactions of certain drugs or diseases that mask a clinical datum of a concurrent disease, that would otherwise improperly rule out the latter due to the apparently missing (masked) clinical datum; (3) establishing whether concurrent diagnoses are related or not.

Recommending empiric treatment, diagnosis by exclusion, and deferred diagnosis for some clinical situations, are also important heuristic aspects of our algorithm.

### Preserving High Quality of Medicine

Although a patent specification must be “a full, clear, concise, and complete description” of the invention, it is important to understand the actual role of the computer in current diagnostic medicine. The United States has available one of the best medical science in the world; however, despite this great technological progress, the social aspects of medicine have declined. First, health management organizations (HMOs) have deteriorated quality of care by pressing physicians to see patients in a shorter time and restricting the request of costly tests and number of tests. General practice physicians were degraded to “gate keepers” ordered to distribute patients among specialists; Medicare reimbursement was reduced for the former and increased for the latter. Later, the tide changed; general practice physicians were pressed to severely limit referrals to the more expensive specialists. As this crisis deepens, we encounter prior art promoting a medicine without physicians: patients that answer prefabricated medical questionnaires and physically examine themselves, sending their observations over the Internet to a computer that is expected to return a diagnosis and treatment advise. Computers provide amazing data storage capacity and data processing speed, but obviously cannot replace physicians, especially regarding patient-physician rapport, physical examination, recommendations, clarifications, human understanding, humility, sympathy, reassurance, compassion, hope, consolation, and solutions to ethical problems. Our computer program is an assisting—not a replacing—tool for physicians; its database aims to comprise all known diseases and clinical data, and precludes overlooking a disease that might afflict a patient. It aspires to replace medical specialists where they are not readily available.

### SUMMARY

This algorithm and computer program emulate the diagnostic reasoning of a clinician. Accurate and efficient, it concludes only those final diagnoses that agree with the diseases that actually afflict a patient, instead of ending with a list of multiple hypothetical competing diagnoses, as most of prior art does. Clinical data manifested by a patient are matched with all disease models, which comprise all clinical data that a disease can potentially manifest. Matched disease models become potential diagnoses which integrate a differential diagnosis list; probability of each diagnosis is calculated with a novel procedure that we call Mini-Max Procedure. This procedure simultaneously processes clinical data present and clinical data absent, using the positive predictive value of clinical data present to increase probability and the sensitivity of clinical data absent to reduce probability. The probability of a diagnosis is considered equal to the maximum positive predictive value of all clinical data present that support the diagnosis, circumventing more complex and inaccurate prior art methods. The mini-max procedure also identifies concurrent diseases. Bayes formula, because of its inability to properly process interdependent clinical data and concurrent diseases, is used with modifications that overcome these limitations. An important function of the algorithm is to recommend at each diagnostic step, the best cost-benefit clinical data next to investigate in a patient. This enables a quicker and more economical achievement of final diagnoses, especially significant in an era of restrictive measures and physician efficiency ratings by health maintenance organizations (HMOs). Furthermore, the algorithm can simultaneously recommend several best cost-benefit clinical data, avoiding the need to contact the patient after the result of each single test is obtained. Interactions of drugs and concurrent diseases with clinical data of the primary disease are detected, precluding ruling out of serious diseases due to this masking effect. Overlooking of important diagnoses is precluded by search for diagnoses that are causally related with the primary final diagnosis. This algorithm diagnoses clinical forms of disease and complex clinical presentations, where diseases, syndromes, complications, and other clinical entities coexist in a single patient. The algorithm is straightforward, logical, and mathematically simple when compared to other attempts in this field. To simplify and facilitate algorithm implementation and updating, we have avoided complicated tools of artificial intelligence, such as causal, hierarchical, and probabilistic trees and networks. Heuristic restrictions preclude excessive proliferation of clinical data and diagnoses. Because it is expressed in natural language, it is readily understandable and user friendly.

### DRAWINGS OF FLOWCHART

### Figures

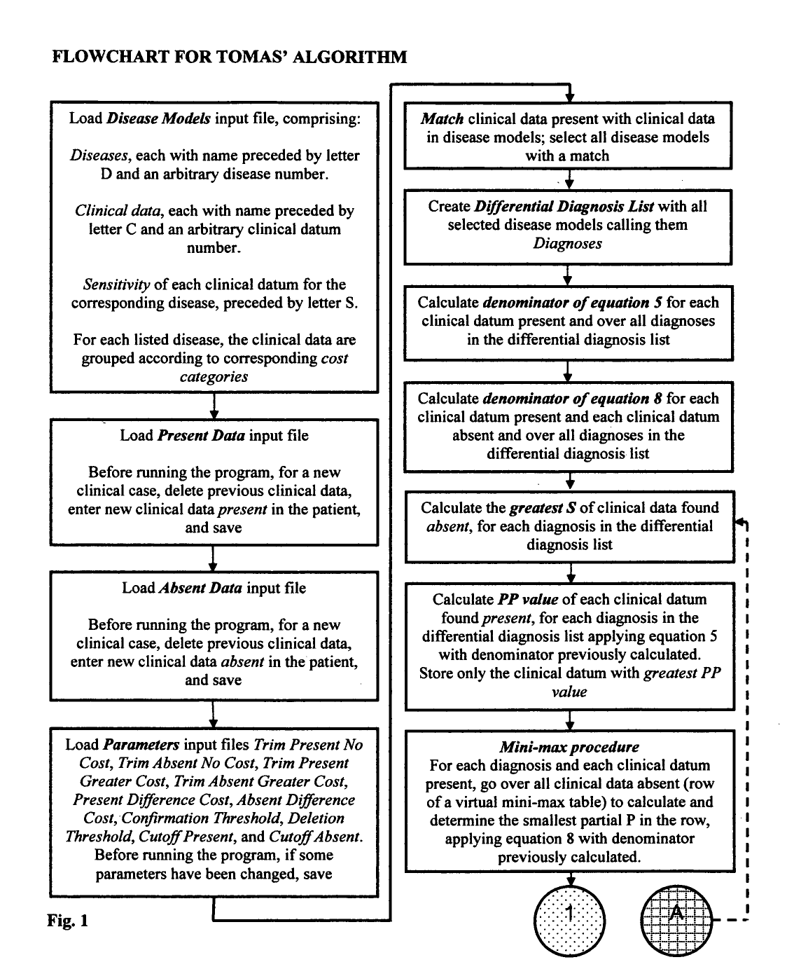

FIGS. 1 through 5 depicts the flowchart of our main computer program—Diagnostic Program.

FIG. 1. Depicts the initial steps of the diagnostic algorithm and program: creation of Disease Models; creation of the input files Present Data and Absent Data where clinical data present and clinical data absent in the patient are respectively entered in the computer; creation of Parameters input files Trim Present No Cost, Trim Absent No Cost, Trim Present Greater Cost, Trim Absent Greater Cost, Present Difference Cost, Absent Difference Cost, Confirmation Threshold, Deletion Threshold, Cutoff Present, and Cutoff Absent, meant to reduce the quantity of recommended best cost-benefit clinical data next to investigate in the patient. The following steps of FIG. 1 search a match of clinical data present in the patient with clinical data in the diseases models, integrating a differential diagnosis list with those disease models that fulfill this match; these diseases are now renamed potential diagnoses. Next steps refer to probability P computation for each diagnosis in the differential diagnosis list, based on our mini-max procedure, explained in great detail in the DESCRIPTION of this patent application.

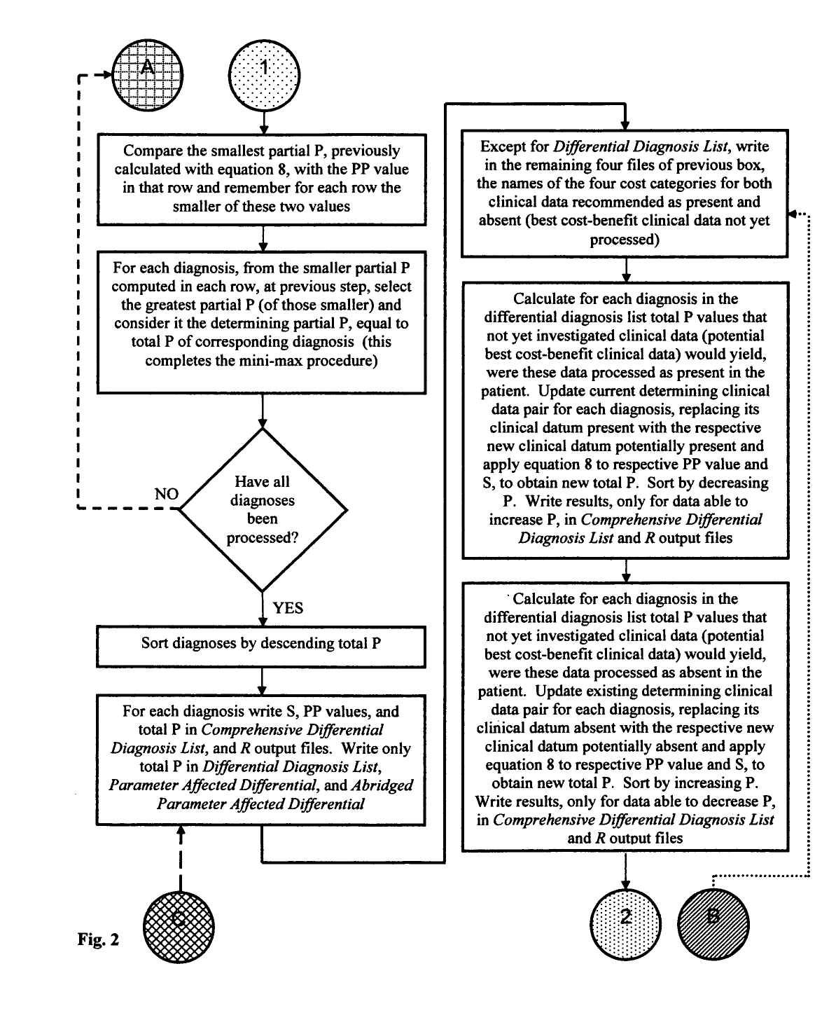

FIG. 2. Depicts the completion of the mini-max procedure, sorting diagnoses by descending total P; creation of diverse output files: Comprehensive Differential Diagnosis List, R output files, Differential Diagnosis List, Parameter Affected Differential, and Abridged Parameter Affected Differential, recommending best cost-benefit clinical data to investigate sorted by cost categories, computing prospective probability P of corresponding diagnosis, were these data found present or absent in the patient.

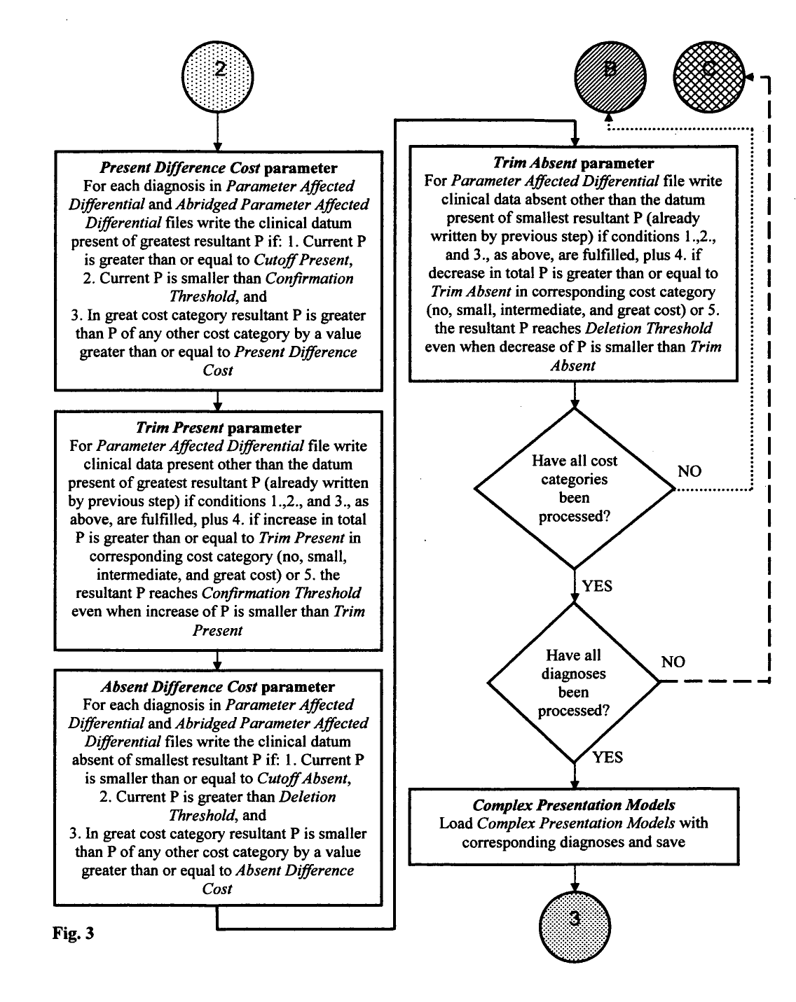

FIG. 3. Depicts the divers parameters mentioned in FIG. 1, devised to reduce the number of best cost-benefit clinical data to investigate, and creation of Complex Presentation Models.

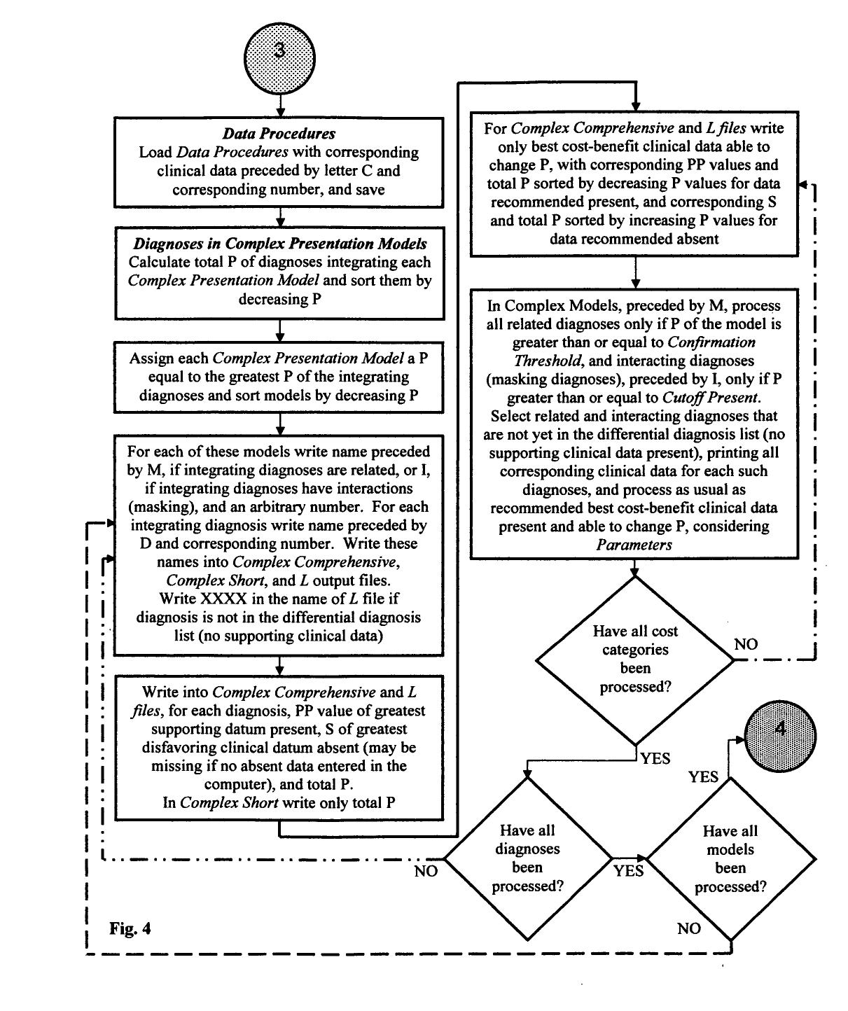

FIG. 4. Depicts grouping of clinical data by procedures necessary to obtain them; integration of diverse types of Complex Presentation Models: Comprehensive Complex, Complex Short, and L output files, computing probability P of diagnoses comprised by these models, considering related diagnoses, and diagnoses with interaction (masking).

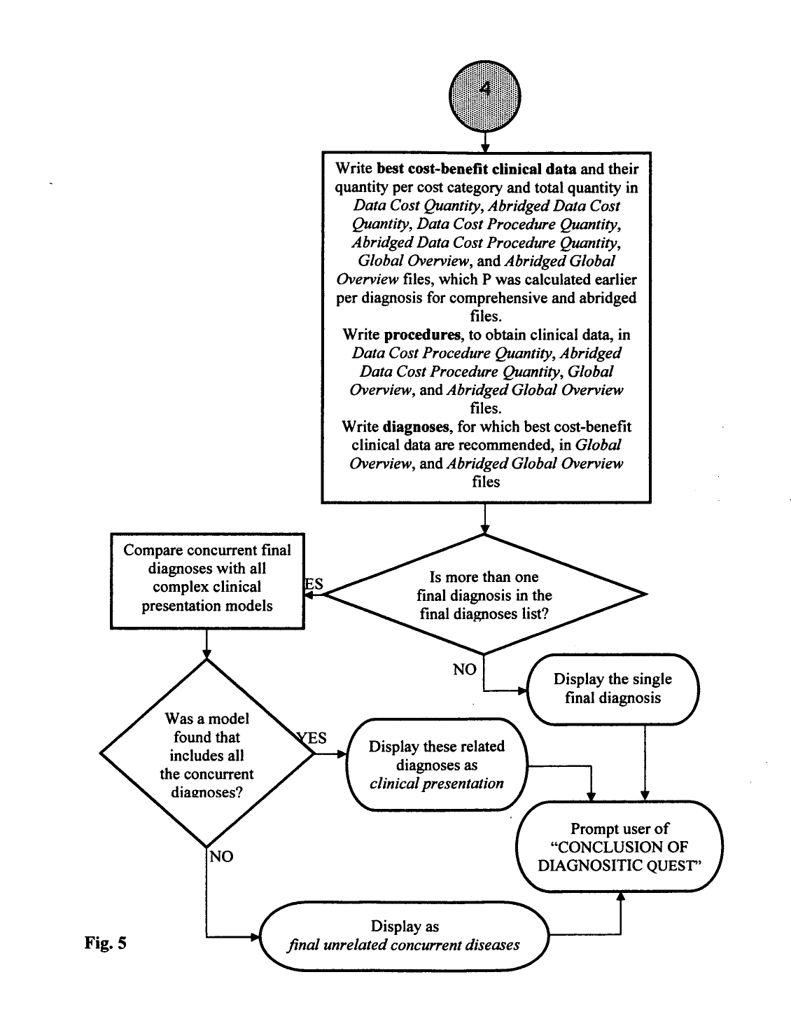

FIG. 5. Depicts integration of diverse output files recommending best cost-benefit clinical data: Data Cost Quantity, Abridged Data Cost Quantity, Data Cost Procedure Quantity, Abridged Data Cost Procedure Quantity, Global Overview, and Abridged Global Overview files, displaying the quantity of clinical data to investigate, the procedure and cost to obtain them, and the diagnoses for which they are investigated. Finally, displaying the end result of the diagnostic quest.

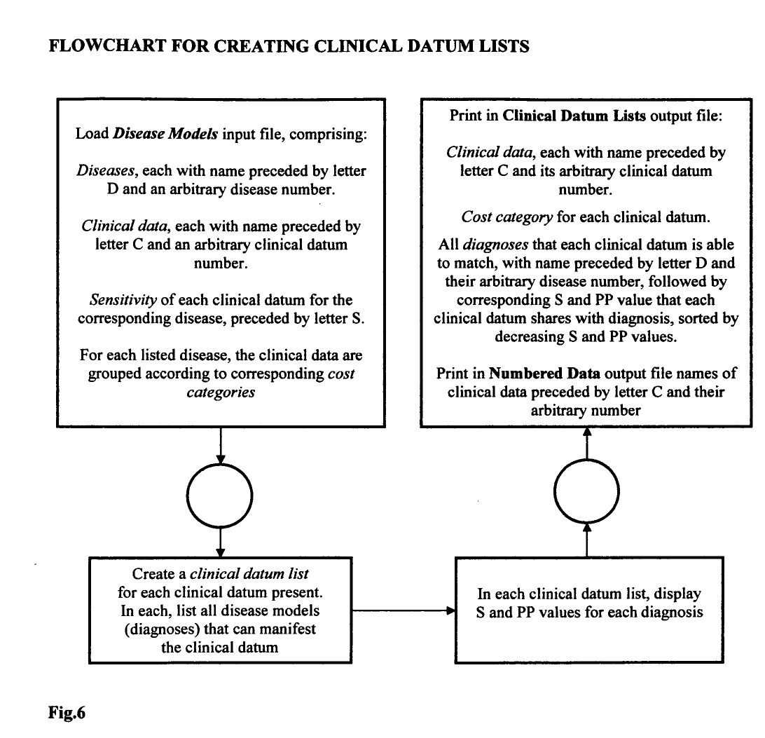

FIG. 6. Depicts the flowchart of our auxiliary program—Datum Program—that creates clinical datum lists reciprocating the information of the disease models. While each disease model in the database comprises all clinical data that a specific disease can potentially manifest, each clinical datum list comprises all diseases able to manifest a specific clinical datum. Diagnoses in each clinical datum list display the sensitivity (S) and the positive predictive value (PP value) that each diagnosis shares with the clinical datum heading this list.

DETAILS OF FLOWCHART are provided by the boxes in the Figures and in the description that follows.

### DESCRIPTION

To understand the present patent application, basic medical and computational concepts must be recalled, and the entire algorithm, involving many novel aspects, needs to be explained. Undoubtedly, the mini-max procedure with its properties, core of my algorithm, is entirely novel.

Some confusion and a lack of consensus exist concerning medical terminology; definitions provided by medical dictionaries often are incomplete or incorrect. The terminology used in this application represents a compromise among current usage, our personal experience, and applicability to our computer algorithm.

### Medical Concepts and Terminology

Disease is a condition in which physicochemical parameter values are out of range and health qualities, such as well-being, harmony in all body functions, and the ability to establish and fulfill goals in life, are altered; in other words, a malfunctioning of the organism. It typically leads to structural changes or lesions.

Etiology is the discipline that studies the causes of diseases; sometimes, the term is used as a synonym for cause of a specific disease.

Pathogenesis is the mechanism by which the cause of a disease produces lesions.

Pathophysiology is the study of the mechanisms by which a lesion causes abnormal function. Abnormal functioning is evidenced by three types of clues:
1. Symptoms, in the strict medical sense, are subjective clues (e.g., pain, nausea, vertigo) that the patient experiences. These clues are revealed by the patient during history taking.
2. Signs are objective clues (e.g., jaundice, swelling, wheezing) that a clinician detects during steps of the physical examination: inspection (observing the patient); palpation (feeling the shape, temperature, consistency, tenderness, of organs); percussion (tapping and listening to the elicited sound); auscultation (listening to sounds produced by organs); and other maneuvers. A patient may or may not be aware of his signs.
3. Results of tests, studies, or procedures are clues obtained through laboratory tests, electrocardiograms, X-ray images, computed tomograms, sonograms, endoscopies, and other techniques.

Various synonyms (data, features, manifestations, traits, attributes, findings) are given to these clues that encompass symptoms, signs, and results of tests, studies, and procedures. We use the term clinical datum (pl. DATA) to refer to any of these clues.

Clinical data tend to cluster into characteristic patterns called syndromes (“running together”). The clinical data that a syndrome comprises typically result from pathophysiologic mechanisms that originate in a common lesion. From the clinical data, tracing back these mechanisms leads to the diagnosis of the lesion.

Concurrent diseases are those that simultaneously afflict a single patient. We call them unrelated concurrent diseases, when the concurrence is random and one disease is completely independent from the other; related concurrent diseases, when dependence exists.

Complication is a secondary disease or medical condition that is a consequence of a primary disease. Typically, a lesion of the primary disease conditions the action of a secondary cause producing the complication. Example: an ingrown nail (primary disease) that provokes a wound (primary lesion), which allows the entry of bacteria (secondary or added cause) provoking infection (secondary disease, which is the complication of the primary disease).

A specific disease may present with diverse clinical pictures involving combinations of clinical data, syndromes, or complications. This diversity is referred to by several terms such as clinical form, clinical presentation, stage, or degree. We were unable to find formal definitions or clear-cut differences between these terms, some of which overlap.

Clinical form: one of the diverse constellations of clinical data manifested, resulting from a single cause or type of lesion. An acute form, displays symptoms that appear suddenly and briefly evolve toward a cure, chronicity, or death (e.g., viral hepatitis). A chronic form has a protracted course (e.g., rheumatoid arthritis). Some forms depend or lesion localization (e.g., pulmonary, intestinal, renal, or genital tuberculosis). Other forms depend on lesion characteristics (e.g., fibrotic, caseous, miliary, or cavitary tuberculosis).

Complex clinical presentation: we reserve this term for cases where two or more final diagnoses are needed to account for all manifested clinical data. For example, coronary artery disease, acute myocardial infarction, congestive heart failure, shock, and thromboembolism in a single patient.

Stage refers to the change of clinical data a disease presents over time. An example is syphilis that progresses through 3 stages, each with totally different syndromes that appear as if they pertain to unrelated diseases.

Degree refers to severity and often is related to duration and progression (stage) of the disease. Examples are congestive heart failure degrees I, II, III, and IV.

Clinical entity: a generic term for any element of a complex clinical presentation, such as a cause, lesion, syndrome, complication, disease, clinical form, stage, or degree.

Disease model, as defined in this patent application, is an abstract concept that comprises all clinical data manifested by all patients with a specific disease. A patient typically never manifests all clinical data that his disease potentially can provoke. Integration of a specific disease model with all of its possible manifestations requires the statistical study of a large patient population. Each clinical form, stage, and degree of disease has its own disease model. Because health, death, and iatrogenic diseases are diagnoses that must be established clinically, the corresponding disease models must also be created. All disease models are stored in the database.

Diagnosis is the identification of an abnormal condition that afflicts a patient, based on manifested clinical data.

Any diagnostic algorithm conceived is likely to be based on disease models stored in the computer database or in the physician's memory. Then, all available information from a specific patient is collected and compared with all disease models. When a successful match between the patient's clinical data and those included in a disease model is achieved, the patient's disease has been diagnosed. This interpretation is related to pattern recognition and perhaps is one way the human mind solves the diagnostic problem; artificial intelligence emulates this process with computers.

In the context of our patent application, diagnosis means probability of disease. This definition refers properly to potential diagnosis; whereas the dictionary definition—the identification of an abnormal condition that affects a specific patient, based on manifested clinical data—is what we call a final or confirmed diagnosis. Unless otherwise qualified, diagnosis will denote potential diagnosis.

Sometimes, the terms disease and diagnosis are wrongly interchanged: for example pneumonia disease for pneumonia diagnosis and vice versa. A disease is a change in the patient's body, whereas a diagnosis is a physician's mental construct. Contradicting diseases (e.g., hyperthyroidism and hypothyroidism) cannot coexist in a given patient, but these two diagnoses could conceivably exist in the same differential diagnosis list, but with different probabilities, because both diseases may share certain symptoms (e.g., pretibial edema.) This confusion leads some computer program researchers to wrongly eliminate one of these apparently contradicting diagnoses from the differential diagnosis list.

Disease prevalence is the fraction of a population afflicted by a specific disease at a specific time. It also can be interpreted as the likelihood of a person belonging to that population to be afflicted by that disease.

### Principles of our Diagnostic Computer Program

### Disregard Disease Prevalence

Prevalence statistics are of epidemiological importance. However, it may be harmful to include prevalence values when calculating the probability of a patient having a rare disease. This happens because the small prevalence value for such a rare disease could considerably reduce the probability of the corresponding diagnosis, causing it to be improperly ruled out. If a patient has a disease afflicting only one in a million persons, the probability of that diagnosis would be very small, but for him or her it represents one hundred percent. A perfect program should diagnose every possible disease, including those that are rare. After all, we do not need a computer to diagnose a common cold during an epidemic. Furthermore, accurate epidemiological information is difficult to obtain because many disease cases remain unreported. Accordingly, our diagnostic algorithm purposely does not take prevalence into account; this is equivalent to assuming that all diseases occur with the same probability.

### Disregard Qualities of Clinical Data

Clinical data, especially subjective symptoms, typically have diverse non-exclusive qualities. For example, chest pain of angina pectoris typically is retrosternal, radiating to the neck, jaw, and upper extremities; is oppressive, lasting only a few minutes; is exertion related and relieved by nitroglycerine. Some authors confer values to these pain qualities, their chronology, and their evolution. This is correct, when such qualities, powerfully suggest a diagnosis. Nevertheless, our algorithm purposely does not consider such clinical data qualities; we believe that computation of clinical datum qualities is not critical for calculation of probability of a diagnosis. Reasons are: clinical data qualities and chronology are subjective and widely variable; chest pain of angina pectoris sometimes is mild, referred to the upper abdomen, not radiating, is burning, or even absent in patients with diabetes. Accordingly, these qualities may not be reliable. Anxious or hypochondriac patients can imagine such qualities. To confirm angina pectoris, more reliable tests, such as stress ECG and sometimes angiogram are needed, which provide clinical data with greater supporting value that anyway will supersede the oppressive quality of chest pain that has a lesser supporting value. Disregarding these unreliable qualities simplifies the diagnostic process without losing accuracy. It would be difficult, if not impossible, to determine the sensitivity, necessary to calculate the supporting positive predictive value, of each diverse quality that thousand of known clinical data can manifest. In the especial case where the quality of a clinical datum is essential, such as the case of prolonged retrosternal pain for myocardial infarction, this quality can be included as a separate clinical datum in the corresponding disease model.

### Categorical and Probabilistic Reasoning in Medical Diagnosis

Whether a computer program should be more categorical (deterministic) or more probabilistic (mathematical) has been repeatedly discussed in the medical diagnosis literature (Szolovits P and Pauker SG, 1978). We are convinced that a diagnostic algorithm must be predominantly categorical. We will insist in later sections that mathematical formulas like the Bayes theorem do not work well for the intended purpose. Physicians in general have great curiosity; they want to know the why, what, where, and when of a diagnosis. They do not trust black boxes with esoteric formulas that yield a diagnosis; they want to know what is inside the box. They want to know details such as clinical presentation, syndromes, pathophysiology, lesion, pathogenesis, and cause. In addition to diagnosing, an algorithm that provides such details can be useful for teaching and research. A mathematical model that attempts to represent the many complex diagnostic situations and nuances by esoteric formulas, matrices, or vectors is divorced from clinical reality.

### Heuristic Versus Exhaustive Search for Medical Information

Heuristics consider problem-solving methods, based on experience, rules of thumb, insight, or intuition, for simplifying and shortening a computer process, as opposed to an algorithm of exhaustive searching, collecting, and processing of information.

Diagnostic reasoning-whether by physicians or computers—is affected by two opposing forces. On one hand, the more clinical data gathered and potential diagnoses processed, the less likely we are to miss an occult disease that might threaten life or expose health care providers to liability. On the other hand, exhaustive collection of medical information is prohibitive because of the incurred cost—price, discomfort, and risk—and processing burden.

Our diagnostic algorithm is based on the emulation of human medical reasoning; accordingly, it uses many heuristic methods as seen in following sections.

### Synonyms of Diagnoses and Clinical Data

Diagnoses and clinical data terms must be recognized and accepted by the algorithm. A given diagnosis or clinical datum can be referred to by different synonyms. Standard terms for clinical data, for example Medical Subject Headings (MeSH) or Systematized Nomenclature of Medicine (SNOMED) are preferred, but if a synonym is used, the algorithm must be able recognize it. When this does not happen, the user is notified and he is prompted to try another synonym. This synonym problem is addressed in the RECONSIDER program (Blois, et al, 1981); the idea of recognizing synonyms is not novel, but the manner in which our algorithm addresses it is novel.

Our program, lists each diagnosis in the database, preceded by a letter D (for Diagnosis) followed by a number up to four digits (Dxxxx <name of diagnosis>), and lists each clinical datum, preceded by a letter C (for Clinical datum) followed by a number up to four digits (Cxxxx <name of clinical datum>). The program recognizes and processes the diagnosis and datum by their numbers, but is blind to the name. We include in the database, after the diagnosis number, the most common synonyms for each diagnosis: e.g., D0008 (Addison's disease, primary adrenocortical deficiency) and after the clinical datum number the most common synonyms for each clinical datum: e.g., C0010 (dyspnea, shortness of breath, respiratory distress). With Find function, the user finds the name of the investigated clinical datum—standard name or synonym—copies it with its corresponding number and pastes it in Present Data or Absent Data; the program will recognize the number and process it, whichever the corresponding term is. Synonyms referring to the same clinical datum must also have the same sensitivity (S) for the corresponding disease. We have noticed in some publications that this S was different for similar synonyms, which would yield different results depending on which synonym used. This is precluded in our program, because each clinical datum is followed by the corresponding S, processed with the clinical datum number, unique for any of the listed synonyms.

### Indices of Clinical Data

In our database, three indices are associated with each clinical datum: sensitivity, positive predictive value, and cost.

### 1. Sensitivity (S)

Sensitivity is defined as the conditional probability P of a clinical datum C, given a disease D:

S=P(C|D) (I)

Where: S=sensitivity of clinical datum C for disease D

P(C|D)=probability of clinical datum C, given a disease D

A practical way to calculate S of a specific clinical datum for a given disease is to determine statistically the fraction of patients afflicted by this disease who manifest the clinical datum:

$$Sensitivity ( S ) = \frac{\begin{matrix}Number of disease cases \\ manifesting the clinical datum\end{matrix}}{Total number of disease cases} \qquad ( 2 )$$

Sensitivity can be expressed either as a decimal (e.g., 0.30), or as a percentage (e.g. 30%.)

A given clinical datum can be manifested by more than one disease. Accordingly, both the clinical datum and the disease that manifests it, determine the value of S. This value is stored in the database linked to the corresponding clinical datum and disease model.

If the numerator and denominator of equation 1 are equal, S of the datum will equal 1, which is unlikely, as it requires that all the clinical cases so far reviewed manifested the clinical datum. Otherwise, the numerator will always be smaller than the denominator, S will be less than 1, and an additional clinical case will increase S if the clinical datum is present, or reduce it if the datum is absent. Accordingly, the computer recalculates the sensitivity of each clinical datum each time the database is updated with new cases. The greater the number of cases analyzed, the greater the accuracy of the sensitivity. If a clinical datum never is manifested by a specific disease, its S equals 0 for this disease. When sufficient number of cases have been reviewed, the disease model will include all the clinical data this disease can potentially manifest, and the sensitivities will approach their true values.

### 2. Positive Predictive Value (PP Value)

Positive predictive value is defined as the probability of a specific disease in a patient that manifests a specific clinical datum:

PP value=P(D|C) (3)

Where: P (D|C)=conditional probability P of specific disease D, given specific clinical datum C

Bayes formula calculates conditional probabilities. Thomas Bayes (1702-1761), a theologian and a mathematician, proposed his formula which was posthumously published in 1763. To my knowledge, Ledley and Lusted first employed Bayes formula for the calculation of the probability of a specific diagnosis, given a clinical datum:

$$P ( {D}_{i} \vert C ) = \frac{P ( {D}_{i} ) P ( C \vert {D}_{i} )}{\begin{matrix}P ( {D}_{1} ) P ( C \vert {D}_{1} ) + … + \\ P ( {D}_{i} ) P ( C \vert {D}_{i} ) + … + P ( {D}_{n} ) P ( C \vert {D}_{n} )\end{matrix}} \qquad ( 4 )$$

### Where:

P(Dᵢ)=probability of disease Dᵢ; also called prior probability because it is the probability of the disease before considering clinical datum C
P(Dᵢ|C)=probability of disease Dᵢ, given specific clinical datum C; also called posterior probability of the disease because it is the result of the equation after considering clinical datum C
D₁ . . . Dₙ=all diseases that manifest clinical datum C, including Dᵢ
P(C|D)=probability of clinical datum C, given a disease D; by definition it equals the sensitivity (S) of the clinical datum for this disease D: P(C|D)=S. This is valid for any disease (D₁ . . . Dₙ) that manifests clinical datum C

We explained earlier the reasons why we purposely do not take into account prevalence of diseases, called prior probability of diseases here in Bayesian context. This is equivalent to assuming that all diseases have the same prior probability [P (D)]; accordingly, we can simplify equation 4 by deleting the prior probability of all diseases [P (D₁) . . . P (Dᵢ) . . . P (Dₙ)]. Then, if we replace P (D|C) with PP value (as in equation 3), and P (C|D) with sensitivity (S) (as in equation 1), we obtain the following equation:

$$PP {value}_{i} = \frac{{S}_{i}}{{S}_{1} + … + {S}_{i} + … + {S}_{n}} \qquad ( 5 )$$

### Where:

PP valueᵢ=positive predictive value of the clinical datum for the disease (i) under consideration
Si=sensitivity of the clinical datum for the disease (i) under consideration
S1 . . . Sn=sensitivities of the same clinical datum for all corresponding diseases*
“All corresponding diseases” could either refer only to diseases that manifest the clinical datum, or alternatively to all known diseases. For either of these alternatives, the result will be identical, because S of a clinical datum for a disease that never manifests such datum is zero. Adding zeros to the value of the denominator established by S of the diseases that manifest the datum will neither change the value of the denominator nor the result of the equation.

Equation 5 expresses the PP value as a function of the sensitivity of a clinical datum for a specific disease (Sᵢ) and the sensitivities of the same clinical datum for all diseases (S₁ . . . Sᵢ . . . Sₙ). It enables the computer to calculate PP value of all clinical data for all diseases. Equation 5 shows that Sᵢ (numerator of the right member) and PP valueᵢ are directly proportional.

PP value=1 when the clinical datum is manifested only by the disease under consideration (that is, when for all other diseases S=0); conversely, PP value approaches 0 when the clinical datum is always manifested in all other diseases (that is, when for all other diseases S=1). In remaining situations, PP value takes an intermediate value between 0 and 1.

PP value quantifies how characteristic or exclusive a clinical datum is for a specific disease or diagnosis. According to equation 5, the fewer the diseases that manifest a given clinical datum and the less often this clinical datum is manifested by each of these diseases (the less the S), the greater the PP value of the clinical datum and the probability for the specific diagnosis or disease. For example, the presence of Mycobacterium tuberculosis in sputum is pathognomonic of pulmonary tuberculosis because no other disease manifests this clinical datum; accordingly PP value=1. For us, PP value is the most accurate index of how strongly a clinical datum supports a diagnosis.

Calculated PP values are linked to the corresponding clinical data in the disease models. Because PP values are based on statistically established S stored in the database, they do not depend on specific clinical cases, and therefore can be calculated before the diagnostic program is delivered to the user. These values remain fixed unless new disease models are added to the database or revised statistics change the values of the sensitivities upon which PP values are based. Should such changes occur as a result of an occasional update, all PP values must be recalculated.

Definition of PP value based on equation 5 is rational, simple, accurate, practical, and novel.

For the diagnostic algorithm to function properly, the database must include all currently known diseases. Should a disease be omitted, its disease model will not be created and the sensitivities of the corresponding clinical data will not be available and will miss from the denominator in Equation 5. Consequently, the calculation of PP value of a clinical datum for any other disease will be inaccurate; still worse, the excluded disease will never be included in the differential diagnosis. For this reason the entire diagnostic algorithm can function properly only when models of all known diseases are included in the database. However, we devised a stratagem that overcomes this limitation, enabling to process medical diagnoses that encompasses a reduced realm of diseases corresponding to a specific specialty; this stratagem (OTHER DISEASES and OTHER DISEASES SAME) will be explained later. We tested this stratagem with our prototype new algorithm and program, proving to work nicely.

As a consequence of Ledley and Lusted's efforts, Bayes formula has become extensively used in medical applications. However, when improperly applied in a diagnostic algorithm, as often is the case, it can cause significant inaccuracies.

Bayes formula is valid only when three conditions are fulfilled:
(i) The diseases processed by the formula must be exhaastive: all known diseases that manifest the considered clinical datum must be included in its denominator. If this condition is violated, some clinical datum originated by a disease not included in the formula will distort the calculated result. Accordingly, the calculated probability of the diagnosis under consideration will be incorrect and will adversely affect the differential diagnosis.
   (ii) Clinical data used for calculation of the conditional probability of a diagnosis must be independent: that is, a specific clinical datum should neither favor nor disfavor any other clinical datum of the same disease. In other words, the probability that one clinical datum is manifested by a specific disease, should not depend on the presence of another clinical datum. This is not true in actual clinical cases, where clinical data result from a chain of reactions that originate in a common cause or lesion and are necessarily related. These clinical data configure syndromes that by definition are associations of related clinical data (e.g., jaundice, increased blood bilirubin, and dark urine). Bayes formula often is applied erroneously to interrelated clinical data of a specific disease, violating this condition of independence and yielding an inaccurate result. To solve the problems of independence and incompatibility, so-called Bayesian networks have been devised, but their application to diagnostic algorithms is excessively complicated and hard to compute.
   (iii) The diseases must be incompatible, which means that clinical data justified by one disease cannot be justified by another disease. When concurrent diseases occur, some clinical data may be caused by more than one of them. Because Bayes formula is only capable to calculate probabilities of competing diagnosis, which are incompatible, it is unsuitable to handle concurrent diseases.

Equation 5 that calculates the PP value of clinical data, derives from Bayes formula, when deleting the prior probabilities. This equation does not violate the aforementioned conditions because it includes in its denominator the S of all known diseases capable of manifesting the clinical datum, complying with the exhaustive condition. These S refer to clinical data pertaining to unrelated disease models (as opposed to a specific patient) complying with the independent condition. Because of the incompatible condition of Bayes formula, our algorithm processes concurrent diseases in a way that does not violate this condition, as explained later (MINI-MAX PROCEDURE).

### 3. Cost

Cost in the context of this patent application, involves not only expense, but also risk and discomfort resulting from the required test or procedure. Expense is quantifiable in dollars or any other currency. Risk can be statistically quantified by outcomes of the procedure, although it also depends on operator skill. Discomfort is a subjective feeling that depends in part on the invasiveness of the procedure and in part on patient apprehension, although the latter can be controlled with sedation or anesthesia. Discomfort cannot be expressed as an exact numerical value, but only can be assigned an estimated qualitative level such as none, small, intermediate, or great. Expense, risk, and discomfort—like apples and oranges—cannot be arithmetically combined into an exact overall cost; however, expense and risk can be qualitatively expressed in levels similar to discomfort, to make the latter comparable to the former two. It is practical, and novel, to consider the maximum qualitative level of expense, risk, and discomfort, as representative of overall cost level:

Cost=max(expense,risk,discomfort)

Because cost does not participate in the calculation of the probability of diagnoses, its inexactness is not critical; it is considered only when selecting the most suitable clinical datum to investigate next in the patient.

We assign to each clinical datum one of four overall cost categories: no cost (clinical data typically obtained through medical history and physical examination), small cost (e.g., obtained through routine laboratory analysis, ECG, and other ancillary studies), intermediate cost (e.g., colonoscopy, lymph node excision biopsy), and great cost (e.g., liver biopsy, laparotomy.)

Cost must be compared to the benefit expected to result from acquiring a clinical datum. Benefit has two components: a quantitative component and a qualitative component. The quantitative component depends on the PP value and S of the clinical datum, which in turn determine P of the corresponding diagnoses. PP value of a clinical datum present in a patient tends to increase the P of the corresponding diagnoses; S of a clinical datum absent tends to reduce the P of the corresponding diagnoses. The clinical datum that has the greatest PP value or the greatest S will result in the greatest benefit because it increments the difference between the P of the most likely diagnoses and the P values of those less likely, which are ultimately eliminated from the differential diagnosis list. The magnitude of the increment of the aforementioned difference of diagnostic P values quantifies the benefit of the clinical datum that produces it. The quantitative component of benefit can be determined before actually investigating a clinical datum for presence or absence in the patient, by virtually testing with the algorithm both possible outcomes.

The qualitative component of benefit cannot be quantified; it depends on multiple factors such as patient health status and ability to tolerate the procedure, patient financial status, insurance company approval, prognosis, involved physician liability, and existence of efficacious and available treatments for the diseases listed in the differential diagnosis. Benefit must equal or exceed cost. The evaluation of cost-to-benefit of a clinical datum and the decision to implement a procedure to obtain it must be discussed with and approved by the patient. If the patient is wealthy, is not discouraged by the risk, or can tolerate discomfort, a procedure that incurs a greater cost may be acceptable. Confirmation of an uncertain diagnosis of a potentially life-threatening but treatable disease also may justify implementation of a more costly procedure.

### Ruling In and Ruling Out Diagnoses

A diagnosis is ruled in when it is included in the differential diagnosis; this occurs whenever patient clinical data match at least one clinical datum in the disease model.

A diagnosis is ruled out when it is deleted from the differential diagnosis; this occurs whenever the probability of the diagnosis falls below an empirical threshold. Clinical data that reduce the probability of a diagnosis favor the deletion of this diagnosis from the differential diagnosis.

These statements imply that a diagnosis must be ruled in before it can be ruled out.

When a new patient, for whom no clinical data are known, comes to our attention, we first collect clinical data manifested by the patient. These clinical data present, when matched with disease model clinical data, introduce the respective diagnoses in a differential diagnosis list, gradually incrementing the number of such potential diagnoses. This process is called ruling in diagnoses. The greater PP value of a clinical datum that is present, the more likely the corresponding diagnosis. For example, microhemagglutination for Treponema pallidum test (MHA-TP) is a clinical datum of great PP value for syphilis; accordingly, if positive, it rules in this disease with great probability, because no other disease manifests this clinical datum. A clinical datum that is present, with great PP value, strongly rules in the diagnosis, even if S is small, meaning that this clinical datum is not frequently found, but as it is already present in this case, S is irrelevant. For example, filarias present in a blood sample is a clinical datum with great PP value for filariasis, confirming this diagnosis, despite a small S. On the other hand, a clinical datum present, typically would not favor a diagnosis only because it has a great S, because it simply tells that this clinical datum is frequently manifested by the specified disease, but many other diseases also may manifest it (small PP value). For example, weight loss has a great S for hyperthyroidism, but a small PP value; therefore, to rule in hyperthyroidism, a clinical datum with a greater PP value, such as suppressed thyroid stimulating hormone (TSH) must be investigated. A clinical datum that is present, with small S, typically would not rule in a diagnosis, because it simply means that this clinical datum is rare for the disease, which is not a reason per se to rule in the disease. For example, diarrhea (small S and small PP value) for duodenal ulcer. Accordingly, ruling in a diagnosis relies on clinical data that are present and the greater the PP value the more it will support this diagnosis. S is irrelevant if the clinical datum is present.

Once some diagnoses are ruled in, integrating the differential diagnosis list, we consider clinical data absent in the patient. For example, when we notice that he is a male, we realize that he cannot have an ovarian cancer; because he is young, prostate cancer is unlikely, and so forth. This process is called ruling out potential diagnoses. To rule out a potential diagnosis, we rely on the sensitivity of clinical data that are absent in the patient. The greater the S of a clinical datum that is absent, the less likely the corresponding diagnosis, even if the PP value is great, because the clinical datum is absent. For example, microhemagglutination for Treponema pallidum (MHA-TP) is a clinical datum of great S for syphilis; accordingly, if negative, it rules out this disease because it is positive in essentially all cases of syphilis (false negative tests are rare). As mentioned in the previous paragraph, weight loss is a clinical datum with great S for severe hyperthyroidism, because it is manifested in all such cases. Accordingly, if this clinical datum is absent, the disease is ruled out. A clinical datum that is absent, with small S, has little influence on the probability of the diagnosis, even if PP value is great. For example, filarias negative in blood (great PP value, but small S) for filariasis. Small sensitivity of an absent clinical datum does not rule out the corresponding diagnosis because it only means that the clinical datum is rare for the disease; absence of a rare clinical datum does not exclude a diagnosis. For example diarrhea with small S and small PP value for duodenal ulcer, if absent, does not rule out this diagnosis. Accordingly, ruling out a diagnosis relies on clinical data that are absent and with great S, PP value is irrelevant if the clinical datum is absent.

Summarizing, a clinical datum present rules in the corresponding diagnosis with strength proportional to its positive predictive value (PP value). A clinical datum absent rules out the corresponding diagnosis with strength proportional to its sensitivity (S).

The following table shows how PP value and S of a clinical datum affect the probability P and ruling in or ruling out of a diagnosis according to whether the datum is present or absent in the patient.

### Ruling In and Ruling Out Diagnoses

| Clinical | | | | Effect on | | |
|---|---|---|---|---|---|---|
| datum | PP value | S | P | Diagnosis | Example | Comments |
| Present | Great | Great | Increased | Strongly | MHA-TP test for | Ruling in a |
| | | | | ruled in | syphilis | diagnosis |
| | Great | Small | Increased | Strongly | Filariae in blood | relies on |
| | | | | ruled in | for filariasis | clinical data |
| | Small | Great | Unchanged | Weakly | Weight loss for | present, with |
| | | | | ruled in | hyperthyroidism | great PP |
| | Small | Small | Unchanged | Weakly | Diarrhea for | value; |
| | | | | ruled in | duodenal ulcer | S is irrelevant |
| Absent | Great | Great | Reduced | Strongly | MHA-TP test for | Ruling out a |
| | | | | ruled out | syphilis | diagnosis |
| | Great | Small | Slightly | Weakly | Filariae in blood | relies on |
| | | | changed | ruled out | for filariasis | clinical data |
| | | | according to | | | absent, with |
| | | | value of S | | | great S; |
| | Small | Great | Reduced | Strongly | Weight loss for | PP value is |
| | | | | ruled out | hyperthyroidism | irrelevant |
| | Small | Small | Slightly | Weakly | Diarrhea for | |
| | | | changed | ruled out | duodenal ulcer | |
| | | | according to | | | |
| | | | value of S | | | |

MHA-TP, microhemagglutination for Treponema pallidum, a highly exclusive and sensitive test for syphilis;
PP value, positive predictive value;
S, sensitivity;
P, probability.

Eight combinations are possible—clinical datum present with great PP value, clinical datum present with small PP value, clinical datum absent with great PP value, clinical datum absent with small PP value, clinical datum present with great S, clinical datum present with small S, clinical datum absent with great S, and clinical datum absent with small S. Of these eight combinations, only two are useful—clinical datum present with great PP value and clinical datum absent with great S-because only they can significantly change the P of the corresponding diagnosis; all other combinations are disregarded.

### Operation of our Diagnostic Algorithm

### Initial Clinical Data Collection

The diagnostic process begins with collection of initial clinical data gleaned from the patient's history, physical examination, and prior consultations. These initial clinical data, entered in the computer, are unrefined because we do not yet know their PP values nor S. These values depend on the specific clinical datum collected, but also on its corresponding, but as yet not ruled in diagnoses. Initially, collection is focused primarily on clinical data present because only these can rule in diagnoses. At this early phase, clinical data processing is purely categorical because we have not yet applied any probabilistic calculations. Only after potential diagnoses are selected can the sensitivity and positive predictive value of clinical data be determined, the probability of each diagnosis be calculated, and a differential diagnosis list be created. Then, if warranted, diagnoses can be ruled out by processing clinical data absent.

Two alternative methods exist for collecting and processing clinical data:
1. The comprehensive method collects as many no cost and small cost clinical data as possible, obtained from the history and physical examination and perhaps some ancillary studies. This approach includes clinical data manifested by both apparent and occasionally occult diseases. We prefer this method because the algorithm emulates traditional physician processing of comprehensive initial information, thereby reducing the risk that the algorithm would arrive at an incomplete or even incorrect diagnosis, as exemplified by the abridged method described in the next paragraph.
2. The abridged method begins with the patient's chief complaint, then gradually refines the diagnostic process by investigating new clinical data recommended by the algorithm. The chief complaint is a symptom (e.g., chest pain) or sign (e.g., bleeding) that prompts the patient to seek medical attention. Accordingly, it might be convenient, although not mandatory, to begin the diagnostic process with the chief complaint; if the patient manifests other significant clinical data, they can be processed at the same time. This abridged, managed-care-style medical examination risks overlooking concurrent occult diseases, arriving at an incomplete or incorrect diagnosis. Examples of how insufficient clinical data can lead to an incomplete or incorrect diagnosis are:
   Incomplete diagnosis: dyspnea, distal edema, bibasal pulmonary rales, hepatomegaly, and cardiac gallop rhythm are the clinical data provided to the computer. Without additional information, the algorithm would correctly diagnose congestive heart failure syndrome. The algorithm might conclude that this syndromic diagnosis is the only final diagnosis, overlooking its underlying cause (e.g., myocardial infarction). This would be even more likely if the chest pain of myocardial infarction is masked by concurrent diabetes. Such an incomplete diagnosis could have dismal consequences because treatment primarily should be directed against the cause and secondarily against the resulting syndrome.
   Incorrect diagnosis: headache, fever, vomiting, photophobia, and neck rigidity are the clinical data provided to the computer. An experienced physician would immediately recognize this constellation of clinical data as characteristic of meningitis even before performing a detailed history and physical examination (this is called the “clinical eye”). The algorithm processes sequentially the first clinical data as they are provided (e.g., headache, fever, and vomiting) and might prematurely conclude an incorrect final diagnosis (e.g., acute gastritis), before requesting more characteristic data (e.g., neck rigidity).
   However, experimenting with our new prototype diagnostic program proved that the abridged method may be as accurate as the comprehensive method, because those clinical data not entered initially will be recommended as best cost-benefit clinical data at following iterations of the program, although this may require more program iterations and more respective patient-physician encounters. Diagnoses not selected by the abridged method will be selected by complex clinical presentation function, although only if those diagnoses are related to confirmed diagnoses, implying a somewhat greater risk of missing valid diagnoses.

How many clinical data should be collected? Ideally and theoretically, to avoid missing a patient's disease, all existing clinical data (several thousand) should be investigated for presence or absence in every patient. Realistically speaking, this is impossible because of time and cost restrictions; it would kill every patient and the socioeconomic system.

How can we reconcile these two opposing forces, namely gathering sufficient medical information vs. time and cost restrictions? A perfect solution does not exist, but heuristic methods became handy. We begin by collecting all possible no cost clinical data that are obtainable from the history and physical examination, and perhaps from the low cost ancillary tests. Later on, we will describe and utilize heuristic restrictive methods (e.g., best cost-benefit clinical datum to investigate next, parameters, abridged output files) to avoid the collection of futile clinical data. We also want to check interaction of clinical data and diagnoses so as to preclude overlooking dangerous diseases. Tracing of links among clinical entities, watchful deferral of diagnoses, empirical treatment, and diagnosis by exclusion, are additional useful heuristic methods, as described later.

Our algorithm has built-in safeguards that preclude incomplete or incorrect diagnoses. An application searches for clinical entities related to the final diagnoses, such as causes or complications, and include them in the differential diagnosis to be confirmed or ruled out.

### Selecting Potential Diagnoses

Following initial clinical data collection, the algorithm next must compare each clinical datum manifested by the patient with all clinical data listed in all disease models stored in the database, selecting those disease models that contain matching clinical data. Such disease models represent potential diagnoses that will become the differential diagnosis list. This task involves important difficulties that are, in my opinion, a major reason why a satisfactory diagnostic algorithm has not yet been achieved. On one hand, a disease typically never manifests all clinical data listed in its disease model; also, the cost of obtaining some of these clinical data may be prohibitive. On the other hand, similar clinical data can be manifested by diverse diseases; in other words, most clinical data are not pathognomonic. The algorithm selects all disease models that include one or more clinical data manifested by the patient. Then it must establish whether the selected diagnoses are competing for a single final diagnosis or whether concurrent diseases exist. Later on we will explain how our algorithm deals with this problem. Clinical Datum Lists

Having selected the matching disease models that now represent potential diagnoses, the algorithm generates, for each clinical datum present, a list that has for heading this clinical datum and comprises all potential diagnoses able to manifest such clinical datum.

### Clinical Datum

Matching disease model 1→Potential diagnosis 1
Matching disease model 2→Potential diagnosis 2
..
..
..
   Matching disease model n→Potential diagnosis n

S and PP value of the clinical datum for each potential diagnosis is shown. The diagnoses are sorted by decreasing PP values.

### Examples of Clinical Datum Lists are:

| Cough | S | PP value |
|---|---|---|
| Foreign body aspiration | 1.00 | 0.20 |
| Bronchitis | 0.98 | 0.196 |
| Tuberculosis | 0.70 | 0.14 |
| Lung cancer | 0.50 | 0.10 |
| Lung infarction | 0.40 | 0.08 |
| Pneumonia | 0.20 | 0.04 |
| . | . | . |
| . | . | . |
| . | . | . |

| Hemoptysis | | |
|---|---|---|
| (bleeding from respiratory tract) | S | PP value |
| Tuberculosis | 0.70 | 0.35 |
| Lung cancer | 0.40 | 0.20 |
| Lung infarction | 0.30 | 0.15 |
| Bronchitis | 0.05 | 0.025 |
| Pneumonia | 0.03 | 0.015 |
| . | . | . |
| . | . | . |
| . | . | . |

| Dyspnea (difficulty to breathe) | S | PP value |
|---|---|---|
| Asthma | 0.98 | 0.208 |
| Congestive heart failure | 0.80 | 0.17 |
| Foreign body aspiration | 0.80 | 0.17 |
| Pneumonia | 0.40 | 0.085 |
| Emphysema | 0.39 | 0.083 |
| Carbon monoxide intoxication | 0.22 | 0.047 |
| Lung cancer | 0.20 | 0.042 |
| Lung infarction | 0.19 | 0.04 |
| Tuberculosis | 0.17 | 0.036 |
| Intense anemia | 0.12 | 0.025 |
| . | . | . |
| . | . | . |
| . | . | . |

The numeric values for S and PP value in the above examples were not obtained from actual statistics or calculations.

In a complete clinical datum list, the sum of the PP values equals 1. The fewer diagnoses a clinical datum list comprises, the more the clinical datum supports those diagnoses and the greater the corresponding PP values. When a clinical datum list contains only one diagnosis, PP value=1, meaning the clinical datum is exclusive or pathognomonic for this diagnosis. Conversely, the more diagnoses a clinical datum list comprises, the less the clinical datum supports those diagnoses, and the smaller their corresponding PP value.

### Differential Diagnosis List

This step creates a differential diagnosis list that comprises potential diagnoses transferred from the clinical datum lists. How many diagnoses should be included in the differential diagnosis list? Again, a concern is avoiding an excessive number of diagnoses, but on the other hand not excluding some that might represent a true disease afflicting the patient. Diverse methods can be followed regarding which of all the diagnoses selected in the entire set of clinical datum lists should be included in the differential diagnosis list.

Our algorithm demands what we call the all-inclusive method that includes in the differential diagnosis list all diagnosis listed in all clinical datum lists, without repeating similar diagnoses; in other words, all the potential diagnoses selected thus far. This method generates a relatively long differential diagnosis list and involves the burden of investigating the presence or absence of clinical data related to each diagnosis. However, this burden is not problematic because this method is applied at an early diagnostic stage when collected initial clinical data are already known to be present or absent, and the number of matched diagnoses is not excessive. Later, heuristically restrictive tools such as the best cost-benefit clinical datum to investigate next (to be explained) will further limit proliferation of clinical data and diagnoses.

Probability of Diagnoses. Mini-Max Procedure

At this point we have a differential diagnosis list. Next, the algorithm must determine which of these potential diagnoses will become one or more final diagnoses.

We devised a procedure for calculating P of a diagnosis by combining the PP value of clinical data when present (favoring a diagnosis) with the S of clinical data when absent (disfavoring a diagnosis). We call it the Mini-Max Procedure. This is the most important and novel aspect of our medical diagnostic algorithm, and main claim for application of a patent. In successive steps, we will explain this procedure with examples.

### Step 1. Process Clinical Data Present

To establish the value of P, prior art programs add, subtract, multiply, or average the sensitivities, specificities, predictive values, estimated values of clinical data supporting a diagnosis, or iterate Bayes formula with each additional clinical datum. These approaches have flaws.

For example, jaundice, dark urine, and increased direct serum bilirubin are clinical data related by similar pathophysiologic mechanisms generated by a single lesion: biliary tract obstruction. Were we arithmetically to combine the individual PP values of these three equivalent and “redundant” clinical data, P of diagnosis biliary tract obstruction would be improperly increased thereby providing an undue advantage to this diagnosis, as compared to competing diagnoses. Furthermore, assume that an endoscopic retrograde cholangiopancreatography (ERCP)—a clinical datum that alone has a PP value of 1—confirms common bile duct-obstructing gallstones. If we add the PP value of other supporting clinical data values, the P of the diagnosis obstructing gallstones would exceed 1, which is probabilistically impossible.

With our algorithm, the PP value of gallstone obstruction, which equals 1, supersedes all other clinical data with lesser PP value (jaundice, dark urine, increased serum bilirubin) because—whether present or absent—they would not change the diagnosis of obstructing gallstones already confirmed by ERCP.

Accordingly, we consider that the greatest PP value of these related clinical data better represents the probability of the diagnosis than any arithmetical combination of the individual values. The algorithm determines P for a specific diagnosis by selecting the greatest PP value that supports this specific diagnosis from all PP values in the entire set of clinical datum lists. The selected greatest PP value equals the P value for this diagnosis.

Pᵢ=max(PP value₁ . . . PP valueᵢ . . . PP valueₙ) (6)

### Where

Pᵢ=probability of the diagnosis under consideration
max=maximum of
PP value₁, . . . PP valueᵢ . . . PP valueₙ=positive predictive values of clinical data present, that support the diagnosis (i) under consideration

The algorithm then iterates the same routine to determine the P of each diagnosis in the differential diagnosis list.

### EXAMPLE

a patient presents with cough, hemoptysis, dyspnea, expectoration, and Mycobacterium tuberculosis (Mycobacterium TB) in sputum. Five clinical datum lists are generated:

| Cough | S | PP value | Hemoptysis | S | PP value |
|---|---|---|---|---|---|
| Pulmonary tuberculosis | 0.80 | 0.276 | Pulmonary tuberculosis | 0.40 | 0.222 |
| Pulmonary embolism | 0.50 | 0.172 | Pulmonary embolism | 0.60 | 0.333 |
| Bronchiectasis | 0.90 | 0.310 | Bronchiectasis | 0.30 | 0.167 |
| Lung cancer | 0.70 | 0.241 | Lung cancer | 0.50 | 0.278 |

| Dyspnea | S | PP value | Expectoration | S | PP value |
|---|---|---|---|---|---|
| Pulmonary tuberculosis | 0.20 | 0.148 | Pulmonary tuberculosis | 0.80 | 0.417 |
| Pulmonary embolism | 0.50 | 0.370 | Pulmonary embolism | 0.02 | 0.010 |
| Bronchiectasis | 0.05 | 0.037 | Bronchiectasis | 0.90 | 0.469 |
| Lung cancer | 0.60 | 0.444 | Lung cancer | 0.20 | 0.104 |

| Mycobacterium TB | S | PP value |
|---|---|---|
| Pulmonary tuberculosis | 0.70 | 1.000 |
| Pulmonary embolism | 0.00 | 0.000 |
| Bronchiectasis | 0.00 | 0.000 |
| Lung cancer | 0.00 | 0.000 |

S values in the above example are for demonstration purposes only and do not represent actual statistics. PP values were calculated by applying equation 5 to these S values. We assume that only the four listed diagnoses exist and that any of them could account for the five clinical data.

In the entire set of clinical datum lists, the greatest PP value for pulmonary tuberculosis is 1.000 and equals P for this diagnosis. Similarly, 0.370 for pulmonary embolism, 0.469 for bronchiectasis, and 0.444 for lung cancer.

A differential diagnosis list is generated, with each diagnosis showing the respective P that competes with the P of the other diagnoses for a final diagnosis. The diagnoses are sorted by decreasing P values.

| Differential Diagnosis List | P |
|---|---|
| Pulmonary tuberculosis | 1.000 |
| Bronchiectasis | 0.469 |
| Lung cancer | 0.444 |
| Pulmonary embolism | 0.370 |

These P values do not yet satisfy thresholds that enable meaningful selection of a final diagnosis (how such thresholds are determined will be explained later.) To satisfy this threshold requirement, the algorithm automatically determines which additional best cost-benefit clinical datum (to be explained later) should next be investigated for its presence or absence. Fever is first recommended for investigation, followed by a pulmonary cavity lesion:

| Fever | S | PP value |
|---|---|---|
| Pulmonary tuberculosis | 0.70 | 0.636 |
| Pulmonary embolism | 0.30 | 0.273 |
| Bronchiectasis | 0.00 | 0.000 |
| Lung cancer | 0.10 | 0.091 |

| Cavity | S | PP value |
|---|---|---|
| Pulmonary tuberculosis | 0.60 | 0.600 |
| Pulmonary embolism | 0.00 | 0.000 |
| Bronchiectasis | 0.10 | 0.100 |
| Lung cancer | 0.30 | 0.300 |

Were fever and a pulmonary cavity lesion also present, we would now have a total of seven clinical datum lists. Were the PP value associated with any of these diagnoses in these 2 new clinical datum lists to exceed the P of the same diagnosis in any of the prior 5 clinical datum lists, that greater PP value would replace the existing P.

PP value of a clinical datum present can only increase the probability of a diagnosis (equation 6.)

### Step 2. Process Clinical Data Absent

Earlier, we presented a rational explanation and example of why we believe that the greatest PP value of all the clinical data present that supports a specific diagnosis equals the P of this diagnosis. This is consequent to the fact that clinical data present are related by a common lesion or cause; arithmetically combining the PP value of these clinical data excessively increases this P. We also mentioned how the S of a clinical datum absent typically reduces the P of the corresponding diagnosis. To reduce P of the corresponding diagnosis, some prior art arithmetically combine the S of all absent clinical data, or sequentially apply Bayes formula to the each S of such clinical data. We observed that this procedure excessively decreases the P of the diagnosis to a value that might incorrectly rule out the corresponding disease. For this reason, to reduce the P of a diagnosis, we use only the greatest S of the all clinical data absent corresponding to this diagnosis. This approach for disfavoring a diagnosis is less intuitive than using the greatest PP value of clinical data present for supporting a diagnosis. Clinical data absent are not related by a common lesion or cause; however, they might be related by a specific characteristic of patient's body that is responsible for the failure to react, or the cause is insufficient to evoke all potential clinical data. This common denominator justifies considering only the datum absent of greatest S as the representative of all clinical data absent. The next example supports this approach:

Consider again a patient with a suspected common bile duct obstruction by gallstones. An endoscopic retrograde cholangiopancreatography (ERCP) in this case was negative—i.e., no gallstones were present in the common bile duct, an absent clinical datum of great S (close to 1) for the mentioned diagnosis. To rule out this diagnosis, it is unnecessary to consider additional clinical data absent of lesser S, such as right upper abdominal pain or vomiting.

So far, we have explained how clinical data present and their associated PP values determine the P of a diagnosis. Now we will explain how clinical data absent and their associated S values further influence this P.

Originally, we tried this equation:

P_{S}=PP value×(1−S) (7)

### Where

PP value=probability of a diagnosis before considering the sensitivity of a clinical datum absent; this probability equals the greatest positive predictive (PP value) value of all clinical data present that support the same diagnosis (equation 6)
P_{S}=probability of the same diagnosis after considering the sensitivity (S) of a clinical datum absent pertaining to the same diagnosis
S=sensitivity of a clinical datum absent pertaining to the same diagnosis; when more than one clinical datum absent refers to this diagnosis, equation 7 should be applied to the greatest S among these clinical data absent
   With equation 7, the greater the S of a clinical datum absent, the more it reduces the P of a diagnosis.

Let's assume that fever in a previous example, were investigated and found absent and let's apply equation 7. Mycobacterium tuberculosis was found in the sputum, a clinical datum present with a PP value=1, which confers a P=1 to tuberculosis, confirming this diagnosis. Next, our example considers fever, a clinical datum absent with S=0.7. Applying equation 7, we obtain:

P_{S}=PP value×(1−S)=1×(1−0.7)=0.3

Note that the absence of fever decreases PP value=1 to P_{S}=0.3. It is unacceptable that a relatively unimportant clinical datum such as fever should cause a substantial decrease in P, which tends to rule out the already confirmed diagnosis of tuberculosis.

To temper this unacceptable decrease in P caused by equation 7, we instead use:

$${P}_{i} = \frac{PP {value}_{i} ( 1 - {S}_{i} )}{\begin{matrix}PP {value}_{1} ( 1 - {S}_{1} ) + … + \\ PP {value}_{i} ( 1 - {S}_{i} ) + … + PP {value}_{n} ( 1 - {S}_{n} )\end{matrix}} \qquad ( 8 )$$

### Where

Pᵢ=probability of a diagnosis (e.g., tuberculosis)
PP valueᵢ=positive predictive value of the clinical datum present (e.g., Mycobacterium tuberculosis in sputum for tuberculosis)
Sᵢ=sensitivity of the clinical datum absent (fever for tuberculosis)
PP value₁ . . . PP valueᵢ . . . PP valueₙ=positive predictive value of the same clinical datum present (Mycobacterium tuberculosis) for each respective diagnosis in the differential diagnosis list (4 diagnoses per our example)
S₁ . . . Sᵢ . . . Sₙ=sensitivity of the clinical datum absent (fever) for each respective diagnosis in the differential diagnosis list (4 diagnoses per our example)

Note that the numerator of equation 8 is identical to the right member of equation 7, and that a denominator has been introduced, the effect of which is to “temper” the result. This denominator comprises several terms, each of which refers to a diagnosis in the differential diagnosis list. Each comprises the PP value of the clinical datum present (Mycobacterium tuberculosis) and the S of the clinical datum absent (fever). These clinical data present and absent remain unchanged for all terms; but their respective PP values and S values change to values associated with each diagnosis. Equation 7 is then applied to these values in each denominator term of equation 8.

Equation 8 is related to Bayes formula, but is here used differently than in other programs; it involves two supposedly independent clinical data—one present and the other absent. Accordingly, Bayes condition of independence is not violated.

Referring to our example of Mycobacterium tuberculosis present in sputum and fever absent, we now must apply equation 8 to calculate the probability of tuberculosis (P_{TB}):

$${P}_{TB} = \frac{PP {value}_{TB} ( 1 - {S}_{TB} )}{\begin{matrix}{PPvalue}_{TB} ( 1 - {S}_{TB} ) + \\ {PPvalue}_{bronchiectasis} ( 1 - {S}_{bronchiectasis} ) + \\ {PPvalue}_{cancer} ( 1 - {S}_{cancer} ) + \\ {PPvalue}_{embolism} ( 1 - {S}_{embolism} )\end{matrix}}$$

Substituting PP values and S values from the clinical datum lists of a previous example into this equation, we obtain

$$\begin{matrix}{P}_{TB} = \frac{1.00 ( 1 - 0.70 )}{\begin{matrix}1.00 ( 1 - 0.70 ) + 0.00 ( 1 - 0.00 ) + \\ 0.00 ( 1 - 0.10 ) + 0.00 ( 1 - 0.30 )\end{matrix}} = \\ = \frac{0.30}{0.30 + 0.00 + 0.00 + 0.00} \\ = 1.00\end{matrix}$$

Note that Equation 8 retains the correct value of P=1 for confirmed tuberculosis, instead of P=0.30, as was obtained with Equation 7.

Equation 8 yields identical result if all PP values of the clinical data present are substituted with the corresponding S′ of the same clinical datum, S otherwise typically used with clinical data absent:

$$\begin{matrix}{P}_{i} = \frac{PP {value}_{i} ( 1 - {S}_{i} )}{PP {value}_{1} ( 1 - {S}_{1} ) + PP {value}_{i} ( 1 - {S}_{i} ) + PP {value}_{n} ( 1 - {S}_{n} )} \\ = \frac{{S}_{i}^{′} ( 1 - {S}_{i} )}{{S}_{1}^{′} ( 1 - {S}_{1} ) + {S}_{i}^{′} ( 1 - {S}_{i} ) + {S}_{n}^{′} ( 1 - {S}_{n} )}\end{matrix}$$

Notice that the value of S′ (sensitivity of the clinical datum present) is not the same as the value of S (sensitivity of the clinical datum absent).

Equation 8, with PP values, yields identical result as with S′ because PP value and S′ of a given clinical datum for a given diagnosis are directly proportional. When all PP values are substituted with the right member of equation 5, equation 8 can be simplified to its substituted form (right member) shown above. This simplification is possible because the sum S₁+ . . . +Sᵢ+ . . . +Sₙ in the numerator and denominator of equation 8 have the same values and cancel each other.

For clarity and consistency, we will retain the original equation 8 for all further calculations, but the substituted form, an alternative embodiment, might be useful for computer programming.

Equation 8 is then iterated to calculate the P that the Mycobacterium tuberculosis-fever clinical data pair confers to pulmonary embolism and the other remaining diagnoses in the differential diagnosis list. PP values and S corresponding to each diagnosis must be substituted in the numerator; the denominator remains unchanged. Equation 8 also normalizes the probabilities of the diagnoses, meaning that their sum (P_{TB}+P_{bronchietasis}+P_{cancer}+P_{embolism}) now equals 1. Referring to our example:

| Mycobacterium TB-Fever | PP value | S | | | P |
|---|---|---|---|---|---|
| Pulmonary tuberculosis | 1.000 × | (1 − 0.70) = | 0.300 (numerator) ÷ | 0.300 (denominator) = | 1.000 |
| Pulmonary embolism | 0.000 × | (1 − 0.30) = | 0.000 (numerator) ÷ | 0.300 (denominator) = | 0.000 |
| Bronchiectasis | 0.000 × | (1 − 0.00) = | 0.000 (numerator) ÷ | 0.300 (denominator) = | 0.000 |
| Lung cancer | 0.000 × | (1 − 0.10) = | 0.000 (numerator) ÷ | 0.300 (denominator) = | 0.000 |
| | | Sum = | 0.300 (denominator) | Sum = | 1.000 |

### Step 3. Create Clinical Data Pairs

A clinical data pair comprises one clinical datum present and one clinical datum absent. Each clinical data pair confers a partial probability to a diagnosis. To calculate the total probability (explained later) of each diagnosis, the Mini-Max Procedure must generate all possible clinical data pairs with all thus-far investigated clinical data present and absent. The number of clinical data pairs generated will equal the number of clinical data present multiplied by the number of clinical data absent.

Returning to our previous example, we had 5 clinical data present (cough, expectoration, hemoptysis, dyspnea, and Mycobacterium tuberculosis) and 2 clinical data absent (cavity and fever), generating a total of 10 clinical data pairs (cough-cavity, cough-fever, hemoptysis-cavity, hemoptysis-fever, dyspnea-cavity, dyspnea-fever, expectoration-cavity, expectoration-fever, Mycobacterium tuberculosis-cavity, and Mycobacterium tuberculosis-fever).

The resultant number of partial P values equals the number of clinical data pairs generated multiplied by the number of diagnoses in the differential diagnosis list. In our example, we had 10 clinical data pairs and 4 diagnoses (pulmonary tuberculosis, pulmonary embolism, bronchiectasis, and lung cancer), yielding a total of 40 partial P.

### Step 4. Create Clinical Data Pair Tables

Then, the 40 partial P values are organized as 10 clinical data pair tables, one table for each clinical data pair. Each table is headed by the clinical data pair; its first column lists the diagnoses of the differential diagnosis list; intermediate columns apply equation 8, and its last column lists the resultant partial P values (see above example of clinical data pair Mycobacterium TB-Fever.)

### Step 5. Calculate Partial P of Each Clinical Data Pair

To calculate the partial P that each clinical data pair confers to each diagnosis in the differential diagnosis list, equation 8 is applied to the PP value of the clinical datum present and the S of the clinical datum absent for each diagnosis.

For our example:

| | PP value | | S | Partial P |
|---|---|---|---|---|
| Cough-Cavity | | | | |
| Pulmonary tuberculosis | 0.276 × (1 − 0.60) = | 0.110 | ÷ 0.730 = | 0.151 |
| Pulmonary embolism | 0.172 × (1 − 0.00) = | 0.172 | ÷ 0.730 = | 0.236 |
| Brochiectasis | 0.310 × (1 − 0.10) = | 0.279 | ÷ 0.730 = | 0.382 |
| Lung cancer | 0.241 × (1 − 0.30) = | 0.169 | ÷ 0.730 = | 0.231 |
| | | 0.730 | | 1.000 |
| Cough-Fever | | | | |
| Pulmonary tuberculosis | 0.276 × (1 − 0.70) = | 0.083 | ÷ 0.731 = | 0.113 |
| Pulmonary embolism | 0.172 × (1 − 0.30) = | 0.121 | ÷ 0.731 = | 0.165 |
| Brochiectasis | 0.310 × (1 − 0.00) = | 0.310 | ÷ 0.731 = | 0.425 |
| Lung cancer | 0.241 × (1 − 0.10) = | 0.217 | ÷ 0.731 = | 0.297 |
| | | 0.731 | | 1.000 |
| Hemoptysis-Cavity | | | | |
| Pulmonary tuberculosis | 0.222 × (1 − 0.60) = | 0.089 | ÷ 0.767 = | 0.116 |
| Pulmonary embolism | 0.333 × (1 − 0.00) = | 0.333 | ÷ 0.767 = | 0.435 |
| Brochiectasis | 0.167 × (1 − 0.10) = | 0.150 | ÷ 0.767 = | 0.196 |
| Lung cancer | 0.278 × (1 − 0.30) = | 0.194 | ÷ 0.767 = | 0.254 |
| | | 0.767 | | 1.000 |
| Hemoptysis-Fever | | | | |
| Pulmonary tuberculosis | 0.222 × (1 − 0.70) = | 0.067 | ÷ 0.717 = | 0.093 |
| Pulmonary embolism | 0.333 × (1 − 0.30) = | 0.233 | ÷ 0.717 = | 0.325 |
| Brochiectasis | 0.167 × (1 − 0.00) = | 0.167 | ÷ 0.717 = | 0.233 |
| Lung cancer | 0.278 × (1 − 0.10) = | 0.250 | ÷ 0.717 = | 0.349 |
| | | 0.717 | | 1.000 |
| Dyspnea-Cavity | | | | |
| Pulmonary tuberculosis | 0.148 × (1 − 0.60) = | 0.059 | ÷ 0.774 = | 0.077 |
| Pulmonary embolism | 0.370 × (1 − 0.00) = | 0.370 | ÷ 0.774 = | 0.478 |
| Brochiectasis | 0.037 × (1 − 0.10) = | 0.033 | ÷ 0.774 = | 0.043 |
| Lung cancer | 0.444 × (1 − 0.30) = | 0.311 | ÷ 0.774 = | 0.402 |
| | | 0.774 | | 1.000 |
| Dyspnea-Fever | | | | |
| Pulmonary tuberculosis | 0.148 × (1 − 0.70) = | 0.044 | ÷ 0.741 = | 0.060 |
| Pulmonary embolism | 0.370 × (1 − 0.30) = | 0.259 | ÷ 0.741 = | 0.350 |
| Bronchiectasis | 0.037 × (1 − 0.00) = | 0.037 | ÷ 0.741 = | 0.050 |
| Lung cancer | 0.444 × (1 − 0.10) = | 0.400 | ÷ 0.741 = | 0.540 |
| | | 0.741 | | 1.000 |
| Expectoration-Cavity | | | | |
| Pulmonary tuberculosis | 0.417 × (1 − 0.60) = | 0.167 | ÷ 0.672 = | 0.248 |
| Pulmonary embolism | 0.010 × (1 − 0.00) = | 0.010 | ÷ 0.672 = | 0.016 |
| Bronchiectasis | 0.469 × (1 − 0.10) = | 0.422 | ÷ 0.672 = | 0.628 |
| Lung cancer | 0.104 × (1 − 0.30) = | 0.073 | ÷ 0.672 = | 0.109 |
| | | 0.672 | | 1.000 |
| Expectoration-Fever | | | | |
| Pulmonary tuberculosis | 0.417 × (1 − 0.70) = | 0.125 | ÷ 0.695 = | 0.180 |
| Pulmonary embolism | 0.010 × (1 − 0.30) = | 0.007 | ÷ 0.695 = | 0.010 |
| Bronchiectasis | 0.469 × (1 − 0.00) = | 0.469 | ÷ 0.695 = | 0.675 |
| Lung cancer | 0.104 × (1 − 0.10) = | 0.094 | ÷ 0.695 = | 0.135 |
| | | 0.695 | | 1.000 |
| Mycobacterium TB-Cavity | | | | |
| Pulmonary tuberculosis | 1.000 × (1 − 0.60) = | 0.400 | ÷ 0.400 = | 1.000 |
| Pulmonary embolism | 0.000 × (1 − 0.00) = | 0.000 | ÷ 0.400 = | 0.000 |
| Bronchiectasis | 0.000 × (1 − 0.10) = | 0.000 | ÷ 0.400 = | 0.000 |
| Lung cancer | 0.000 × (1 − 0.30) = | 0.000 | ÷ 0.400 = | 0.000 |
| | | 0.400 | | 1.000 |
| Mycobacterium TB-Fever | | | | |
| Pulmonary tuberculosis | 1.000 × (1 − 0.70) = | 0.300 | ÷ 0.300 = | 1.000 |
| Pulmonary embolism | 0.000 × (1 − 0.30) = | 0.000 | ÷ 0.300 = | 0.000 |
| Bronchiectasis | 0.000 × (1 − 0.00) = | 0.000 | ÷ 0.300 = | 0.000 |
| Lung cancer | 0.000 × (1 − 0.10) = | 0.000 | ÷ 0.300 = | 0.000 |
| | | 0.300 | | 1.000 |

At this point, we have calculated the partial probability that each clinical data pair confers to each diagnosis.

### Step 6. Create Mini-Max Tables

Now, we must determine the total probability that the partial probabilities mentioned in steps 4 and 5 confer to each diagnosis in the differential diagnosis list. This is achieved by generating a mini-max table for each such diagnosis (see tables on next pages).

The first column of each mini-max diagnosis table lists each clinical datum present.

The second column lists the PP value of each clinical datum present; its bottom cell repeats the greatest of these values, which is the total P of the diagnosis before clinical data absent are considered.

The next several columns show the partial P values that each clinical data pair confers to the diagnosis; the number of these columns equals the number of clinical data absent. The heading of each column shows the clinical datum absent and its S for the diagnosis. Each partial P value is transferred from the clinical data pair table to the mini-max table cell where the clinical data present and absent converge. The bottom cell of each column repeats the greatest partial P value appearing in the column.

The last column repeats the smallest value appearing in each row. The bottom cell of this column, which also is the last cell of the mini-max table, repeats the greatest value of the column; it equals the total P of the diagnosis, after clinical data absent have been considered.

### Mini-Max Table for Tuberculosis

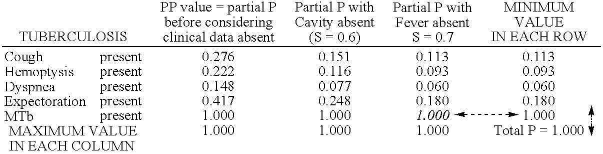

MTb, Mycobacterium tuberculosis
The total probability of tuberculosis at this diagnostic step is the maximum value (1.000) in the last column.

### Mini-Max Table for Pulmonary Embolism

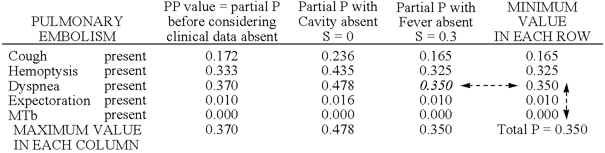

The total probability of pulmonary embolism at this diagnostic step is the maximum value (0.350) in the last column.

### Mini-Max Table for Bronchiectasis

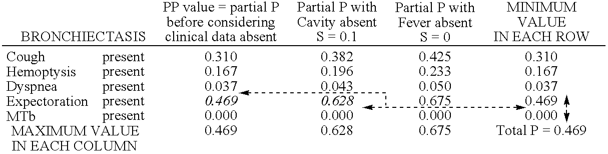

The total probability of bronchiectasis at this diagnostic step is the maximum value (0.469) in the last column. Were the second column not included in the calculation, total P of this diagnosis would be 0.628; see property 4 B of Mini-Max Procedure (page 36).

### Mini-Max Table for Lung Cancer

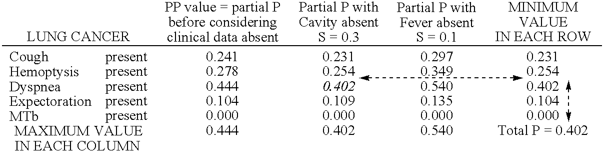

The total probability of lung cancer at this diagnostic step is the maximum value (0.402) in the last column.

### Step 7. Determine Total P of a Diagnosis

The algorithm determines the total P value of a diagnosis based on partial P values and these concepts:
1. A clinical data pair comprises a clinical datum present and a clinical datum absent.
2. A specific clinical data pair (in some clinical data pair tables) is responsible for the total P of a specific diagnosis.
3. In this specific clinical data pair, the clinical datum present has the greatest PP value of all clinical data present and the greatest rule-in effect for the specific diagnosis.
4. In this specific clinical data pair, the clinical datum absent has the greatest S of all clinical data absent and the greatest rule-out effect for the specific diagnosis.
5. Applying Equation 8 to the PP value of the clinical datum present and the S of the clinical datum absent in the specific clinical data pair yields a specific partial P for the specific diagnosis.
6. A specific cell for this specific partial P exists in the mini-max diagnosis table of this specific diagnosis.
   In this specific cell, the clinical datum present (mentioned in 3) converges with the clinical datum absent (mentioned in 4.)
   In this specific cell, the value (italicized) of the specific partial P (mentioned in 5) was transferred from the clinical data pair table to the mini-max table.
   In this specific cell, the value of the specific partial P is at once the smallest in its row and the greatest in its column.
7. We call the specific clinical data pair (mentioned in 2) the determining clinical data pair (because it determines the value of the specific partial P (mentioned in 5.)
8. We call this specific partial P the determining partial P because it determines and equals the value of the total P of the specific diagnosis (see arrows in mini-max tables.)

To find the clinical data pair (determining clinical data pair) responsible for the current total P of a diagnosis, we must backtrack the steps that lead from that pair to the total P. Start at the last cell (total P) of the mini-max table and ascend (following the arrows in any mini-max diagnosis table) to any cell with the same value, then go left on that row until any cell with the same value (the determining partial P) is encountered. The clinical datum present and the clinical datum absent that converge to this cell comprise the requisite clinical data pair (determining clinical data pair); their respective PP value and S values are as shown in the mini-max table.

### EXAMPLE

In the mini-max table of Lung Cancer the last cell shows the current total P (0.402) of this diagnosis. Following the arrows takes us to another cell with the italicized value 0.402, which is the determining partial P. To this cell converge PP value (0.444) of dyspnea present and S (0.3) of pulmonary cavity absent. The clinical data pair dyspnea-cavity is responsible for the current determining partial P and total P (0.402.)

Mini-max diagnosis tables are not based on Bayes formula and therefore circumvent the problem of clinical data independence and disease incompatibility.

### Broken Monotony

Typically, the partial P values in the rows of the mini-max table present a monotone relation; this means that when in one row the partial P value increases or decreases from one cell to the next, in the other rows the changes occur in the same direction. However, sometimes this monotone relation is broken. This is due to the especial interrelation among the diverse S and PP values in the clinical data pairs. Broken monotony has several consequences:
A single determining partial P that is at the same time the greatest partial P of its column and the smallest partial P of its row may no longer exist.
The maximum partial P in the last column and the minimum partial P value in the last row do no longer yield the same value, equal to total P of the diagnosis, as it occurs when monotony is not broken.
A clinical datum of smaller PP value is able to increase the total P more than a clinical datum with greater PP value, violating the rule that the greatest PP value of clinical data supporting a diagnosis equals the total P of this diagnosis (equation 6, page 25).
A clinical datum of smaller S is able to decrease the total P more than a clinical datum with greater S, which does not occur when monotony is preserved.
   Our new diagnostic algorithm ignores broken monotony, selecting one (greatest value of last column in mini-max table) of the two different resultant total P values for the same diagnosis, because their difference in magnitudes is insignificant, and our program proved to remain accurate and efficient.

### Step 8. Update the Differential Diagnosis List

Next, we again sort all diagnoses in the differential diagnosis list, according to decreasing total P values:

| Differential Diagnosis List | Total P |
|---|---|
| Tuberculosis | 1.000 |
| Bronchiectasis | 0.469 |
| Lung cancer | 0.402 |
| Pulmonary embolism | 0.350 |

Note that the total P values of the diagnoses have changed and are more widely dispersed, but they still do not satisfy our thresholds for meaningful selection of a final diagnosis. Accordingly, additional clinical data must be investigated. Then the Mini-Max Procedure must be iterated with each additional clinical datum, and the total P of all diagnoses recalculated, until requirements for conclusion of the diagnostic quest are satisfied.

### Properties of the Mini-Max Procedure:

1. Each additional clinical datum present generates a new row in an existing mini-max table.
2. Each additional clinical datum absent generates a new column in an existing mini-max table.
3. A mini-max table has only one determining partial P cell, the value of which (italicized in the table) is the smallest of its row and the greatest of its column. The clinical datum present and the clinical datum absent that converge to this cell constitute the clinical data pair that originated this partial P that equals the total P of the diagnosis.
4. When an additional clinical datum present is processed with the mini-max procedure, the total P of the diagnosis may increase, depending on its PP value. Typically, when an additional clinical datum absent is processed with the Mini-Max Procedure, the greater its S, the more it decreases the P of the diagnosis. However, exceptions to this rule result due to the particular interrelations that S of a clinical datum absent has with the S of the same clinical datum for the other diagnoses in the clinical data pair table, and from the interaction of the resulting partial P values in the mini-max table. The total P of the diagnosis will either decrease, increase, or remain unchanged:
   A. Total P decreases. Let's concentrate on a clinical data pair table. For a specific diagnosis, the S value of the clinical datum absent is inversely related to its partial P and directly related to the partial P values of the other diagnoses. An additional clinical datum absent typically reduces the total P of a diagnosis if its S is greater than the S of the absent clinical datum in the determining clinical data pair, in turn responsible for the current determining partial P of the diagnosis. If this condition is fulfilled, this new partial P will be less than the current determining partial P and becomes the new determining partial P that equals total P in the mini-max diagnosis table.
   B. Total P increases. The mini-max procedure is not intended to increase the total P of a diagnosis based on clinical data absent. Nevertheless, this occasionally occurs, but only when an initial clinical datum absent is processed; because at this point only one clinical datum absent column is generated, smaller values do not exist in the rows. The greatest partial P value in this column becomes the determining partial P and if it exceeds the current total P, it will replace the latter. Any subsequent clinical datum absent that is processed—regardless of its S value and resulting partial P—can only decrease the total P, because only the smallest partial P in a row can become a determining partial P. If we do not want an initial clinical datum absent to increase the current total P of a diagnosis, then the second column of the mini-max table must be included in the calculation. In this way, we avoid violating the general rule that a clinical datum absent must never increase the total P.

An example is the mini-max table for bronchiectasis (see table page 33), where the total P of this diagnosis would have been 0.628 (italicized) instead of 0.469, were the second column not included in the calculation.
C. Total P does not change, when a clinical datum absent does not fulfil any of the conditions for decreasing or increasing total P. This occurs frequently; furthermore, the total P of a diagnosis is quite resistant to change, especially for diagnoses with a great total P. This is an important advantage of the Mini-Max Procedure, because it precludes ruling out a confirmed diagnosis (strongly supported by clinical data present) by some relatively unimportant clinical datum absent (as seen earlier, in the example of tuberculosis, page 29).
5. The order in which clinical data are processed is irrelevant; it will change only the relative position of the generated new row or column without affecting the total probability of the diagnosis. This commutative order is intuitive and consistent with experience.
6. When an additional clinical datum present or absent is incorporated into a mini-max table, the previously calculated partial P values of the diagnosis in the table remain unchanged and need not be recalculated. Such P values are retained in case a need arises to determine which clinical datum pair generated a partial probability in a cell. The algorithm need remember the values in the last column only. Whenever an additional clinical datum is processed, new clinical data pairs are generated and new partial P values are calculated. The algorithm then compares these new partial P values with the existing partial P values in the last column and calculates the new total probability of the diagnosis.
7. An interesting property of the mini-max procedure is revealed when the sum of the total P of all diagnoses in the differential diagnosis list is substantially greater than 1; it suggests that not all such diagnoses are competing, but that some represent concurrent diseases. The degree of support that a clinical datum gives to a diagnosis is directly proportional to its corresponding PP value. This value can be found in the clinical datum list associated with the diagnosis or in the second column of the mini-max table. If all clinical data predominantly support the same diagnosis, the remaining diagnoses tend to compete and the sum of the probabilities of all diagnoses in the differential diagnosis list is close to 1. When some clinical data predominantly favor one diagnosis and other clinical data predominantly favor another diagnosis, these diagnoses tend to be concurrent; the sum of their probabilities will be considerably greater than 1. Concurrent diagnoses are supported by different clinical data; accordingly, each concurrent diagnosis can by itself attain a probability up to 1. The greater the sum of the probabilities, the greater the number of concurrent diseases.
8. When a clinical datum present with a PP value that approaches or equals 1 strongly supports or confirms a diagnosis, a clinical datum absent—regardless of its S value—cannot reduce the great P that such a clinical datum present confers to the diagnosis. This property also is true for concurrent diagnoses with great probability in the differential diagnosis list. This important advantage precludes a confirmed diagnosis from being ruled out by a relatively unimportant clinical datum absent (see previous tuberculosis example, page 29). However, the P of a diagnosis without a confirming clinical datum present may be reduced by the S of such a clinical datum absent. Retaining diagnoses with great P, while simultaneously ruling out diagnoses with a small P, enables concurrent diagnoses to be distinguished from competing diagnoses in a differential diagnosis list. The manner in which the algorithm processes concurrent diagnoses will be addressed later.
9. Each time an additional clinical datum becomes available, the Mini-Max Procedure recalculates de novo the total P values of the diagnoses, processing all present and absent clinical data.

What happens when a diagnosis with a great P, based on a clinical datum present with a PP value=1, is confronted with an additional clinical datum absent with an S=1? Would the clinical datum present or the clinical datum absent win the rule in/rule out contest? In an actual case, this confrontation would be most unlikely to occur because S=1 means that this clinical data is always present. Furthermore, the Mini-Max Procedure precludes the discredit of a diagnosis with a great total P by a clinical data absent.

### Competing and Concurrent Diagnoses

Total P of each diagnosis is calculated by its corresponding mini-max table, based on PP value of clinical data present, S of clinical data absent, and resultant partial P values, all of which are specific for this diagnosis, allowing a certain independence among diverse diagnoses. This enables that each diagnosis which P reaches a confirmation threshold is declared final diagnosis, irrelevant of how many other diagnoses also reach this threshold; all of such diagnoses are considered concurrent. Competing diagnoses are ruled out, by reaching a deletion threshold.

### Best Cost-Benefit Clinical Datum Next to Investigate

The best cost-benefit clinical datum next to investigate for presence or absence in a patient is an important function that can substantially shorten and reduce the cost of a diagnostic quest by precluding investigation of futile clinical data. This has important socioeconomic implications, especially in this era of managed care, when insurance companies curtail tests and procedures, and when physicians are rated by their proficiency in ordering tests in general. The way our algorithm determines the best cost-benefit clinical datum next to investigate for presence or absence is novel and a claim to be patented; it depends on the Mini-Max Procedure.

Computers are faster and more accurate than the human brain in selecting the most convenient clinical datum next to investigate for presence or absence at each diagnostic step.

As stated earlier, our algorithm recommends the best cost-benefit clinical datum next to investigate, based on cost and diagnostic power.

Because the term best cost-benefit clinical datum next to investigate in a patient is lengthy, we shorten it to best cost-benefit clinical datum.

The best cost-benefit clinical datum function enables us to predict which new clinical datum will most increase or decrease the total probability (P) of a diagnosis, reducing the number of clinical data required to achieve a final diagnosis.

A recommended best cost-benefit clinical datum can be evaluated-fore actually accomplishing the corresponding test or procedure—by virtually considering it either present or absent, while observing whether it improves the diagnostic outcome.

Initial clinical data collection was achieved during the history and physical examination. We accepted whatever clinical data were revealed, without considering their rule-in or rule-out power. Subsequent clinical data collection is more selective, because we have a differential diagnosis list and a better-structured diagnostic process that enables to apply statistical and probabilistic concepts, and choose the best cost-benefit clinical datum, based on cost, positive predictive value, and sensitivity.

To select a best cost-benefit clinical datum, several steps must be followed:

### Step 1. Select Clinical Data Not Yet Investigated in the Patient

The algorithm examines every diagnosis in the differential diagnosis list and selects from its respective disease model all clinical data not yet investigated. These clinical data differ from those initially collected; they are expected to be numerous because each disease model will contribute many new clinical data. However, only clinical data of appropriate cost and either of great PP value or great S need to be investigated for presence or absence.

### Step 2. Organize Clinical Data Not Yet Investigated According to Cost Category, Diagnosis, PP Value, and S

Clinical data not yet investigated are organized according to three hierarchical levels (see diagram on next page). The first level is COST CATEGORIES, comprising four categories: none, small, intermediate, and great. The second level is DIAGNOSES, comprising all diagnoses in the differential diagnosis list, identically repeated in each cost category, in order of decreasing P value. The third level is CLINICAL DATA, comprising two lists that we call PP VALUE LIST and S LIST containing only those clinical data that have a cost similar to the corresponding cost category. Both lists contain the same clinical data, but ordered according to decreasing PP value and decreasing S respectively, and consequently these clinical data are shown with different sequence in each list.

Diagram. Nested Loops for Selecting Best Cost-Benefit Clinical Datum

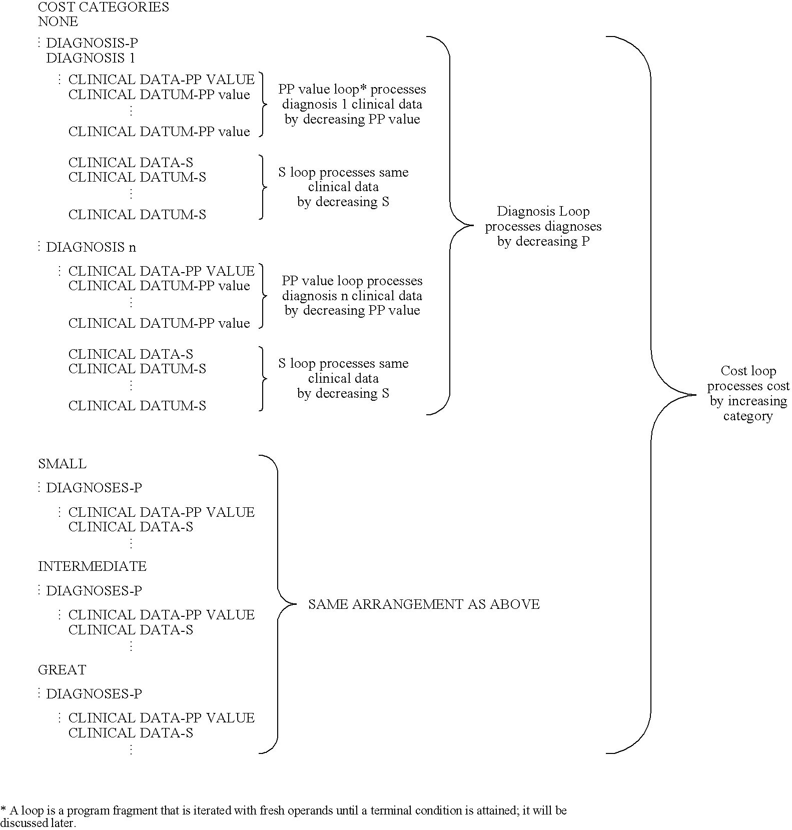

### Step 3. Recommend a New Clinical Datum as Best Clinical Datum Assuming it Present

The algorithm moves to the lowest as-yet-unprocessed COST category, selects the as-yet-unprocessed DIAGNOSIS with greatest P, and from the corresponding PP VALUE LIST, selects the as-yet-unprocessed clinical datum with the greatest PP value. This PP value then is compared to the PP value of the clinical datum present in the current determining clinical data pair. The value of the latter appears in the bottom cell of the second column of the mini-max diagnosis table for this diagnosis, and equals the current P of the diagnosis before processing clinical data absent. New clinical data with equal or smaller PP value can be disregarded because—even if present—they will not change the current P of this diagnosis; then, the algorithm moves to step 4 (page 43). When the new clinical datum has a PP value that exceeds the current P of the diagnosis before considering clinical data absent (bottom cell of second column), the algorithm recommends it as best cost-benefit clinical datum. The user then verifies whether this clinical datum is absent or present. When this clinical datum is absent, it is disregarded, because if able to change the total P of the diagnosis, it will be detected by the S loop at the next STEP 4, which processes clinical data assumed absent. When the recommended best cost-benefit clinical datum is present, the algorithm generates a new clinical datum list headed by this datum, and the current P of the diagnosis before considering clinical data absent assumes the PP value of this new datum. To recalculate the total P of the diagnosis after considering clinical data absent, several new clinical data pairs, combination of the best cost-benefit clinical datum present with each clinical datum absent, are generated; new clinical data pair tables are created and the partial P values for the diagnosis are calculated. A new row with these values is inserted in each mini-max diagnosis table and the total P of the corresponding diagnosis is established.

### EXAMPLE

Assume we need to know whether the clinical datum pulmonary mass as evidenced by X-ray plain films, when present, can increase the current total P of lung cancer (see mini-max table for lung cancer, page 34). The PP value of a pulmonary mass for lung cancer, stored in the disease model in the database, is 0.857. Equation 6 states that the greatest of the clinical data PP values supporting a diagnosis equals the P value of this diagnosis:

$$\begin{matrix}{P}_{lung cancer} = max ( \begin{matrix}PP {value}_{cough ,} PP {value}_{hemoptysis ,} PP {value}_{dyspnea ,} \\ PP {value}_{expectoration ,} PP {value}_{MTb ,} PP {value}_{mass}\end{matrix} ) \\ = max ( 0.241 , 0.278 , 0.444 , 0.104 , 0.000 , 0.857 ) \\ = 0.857\end{matrix}$$

Accordingly, the previous P of lung cancer (0.444), before considering clinical data absent (bottom cell of second column in the lung cancer mini-max table), is increased to its new value of 0.857. The algorithm then generates a new clinical datum list:

| Pulmonary mass | S | PP value |
|---|---|---|
| Lung cancer | 0.9 | 0.857 |
| Pulmonary tuberculosis | 0.1 | 0.095 |
| Pulmonary embolism | 0.05 | 0.048 |
| Bronchiectasis | 0.0 | 0.000 |

To determine the total P of the diagnosis, after considering clinical data absent, the algorithm generates clinical data pair tables and calculates the partial P values for lung cancer:

| Mass-Cavity | PP value | S | | | Partial P |
|---|---|---|---|---|---|
| Lung cancer | 0.857 × | (1 − 0.30) = | 0.600 | ÷ 0.686 = | 0.875 |
| Pulmonary | 0.095 × | (1 − 0.60) = | 0.038 | ÷ 0.686 = | 0.055 |
| tuberculosis | | | | | |
| Pulmonary | 0.048 × | (1 − 0.00) = | 0.048 | ÷ 0.686 = | 0.070 |
| embolism | | | | | |
| Bronchiectasis | 0.000 × | (1 − 0.10) = | 0.000 | ÷ 0.686 = | 0.000 |
| | | | 0.686 | | 1.000 |

| Mass-Fever | PP value | S | | | Partial P |
|---|---|---|---|---|---|
| Lung cancer | 0.857 × | (1 − 0.10) = | 0.771 | ÷ 0.833 = | 0.925 |
| Pulmonary | 0.095 × | (1 − 0.70) = | 0.028 | ÷ 0.833 = | 0.034 |
| tuberculosis | | | | | |
| Pulmonary | 0.048 × | (1 − 0.30) = | 0.034 | ÷ 0.833 = | 0.041 |
| embolism | | | | | |
| Bronchiectasis | 0.000 × | (1 − 0.00) = | 0.000 | ÷ 0.833 = | 0.000 |
| | | | 0.833 | | 1.000 |

In the mini-max diagnosis table for lung cancer, the algorithm generates a new row that shows these partial P value; then, the total P of this diagnosis after considering clinical data absent is established:

Mini-Max Diagnosis Table for Lung Cancer when Pulmonary Mass is Present

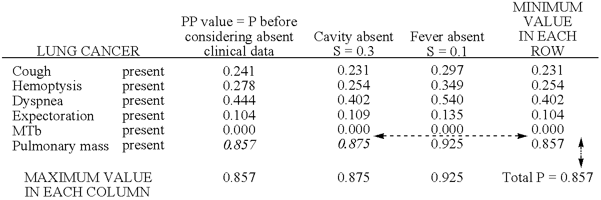

MTb, Mycobacterium tuberculosis

The total P of lung cancer at this diagnostic step is the maximum value (0.857) in the last column. Because of pulmonary mass present in chest X-ray plain films, the total P of lung cancer increased from 0.402 to 0.857.

Notice that except for the last two rows, partial P values shown in all other cells remain unchanged. However, the value and location of the determining partial P cell changed.

### Step 4. Recommend a New Clinical Datum as Best Clinical Datum Assuming it Absent

Typically, the greater the S of a clinical datum absent, the more it decreases the total P of a diagnosis. However, with the mini-max procedure, a clinical datum absent can occasionally increase total P (mini-max property 4 B, page 36).

In the same COST category and DIAGNOSIS in which a new best cost-benefit clinical datum assumed present was processed involving PP value list, the algorithm moves now to the S LIST. From this list, the clinical datum of greatest S, not yet selected, is selected and substitutes the clinical datum absent in the current determining clinical data pair, creating a new clinical data pair.

In our previous main patent application, we described a 3-Step method to determine whether a new clinical datum absent will decrease total P, devised to shorten computer processing time; this method is still valid and could be valuable when the database includes a great number of disease models to be processed. However, after experimenting with our new prototype program we realized that at least for limited diseases models in the database, the 3-step method is unnecessary. We replaced it with a simplified mini-max procedure that continues applying the original method to calculate all the partial P (all the cells) of the mini-max table after entering new clinical data confirmed present or absent in the patient; but for recommending best cost-benefit clinical data it is sufficient to calculate only the determining partial P. This is achieved by directly applying equation 8 to the clinical datum present with the greatest PP value and the clinical datum absent with the greatest S, equivalent to filling only one cell of the new column corresponding to the clinical datum absent. This manner to precede does not compromise the accuracy of the diagnostic process, because the next iteration of the algorithm, after inclusion of new clinical data, will calculate the partial P for all the cells in all mini-max tables (all diagnoses).

When the resulting P of the respective diagnosis, determined with only one partial P in the new column, is smaller than the current P, the clinical datum assumed absent is recommended as best cost-benefit clinical datum. If this new clinical datum is confirmed absent in the patient and entered as such in the computer, a new column will be generated in the mini-max table and all cells of this column will be filled with corresponding partial P, after program iteration, enabling determination of a more reliable total P.

### EXAMPLE

Assume we need to know whether the clinical datum pulmonary mass as evidenced by chest X-ray plain films, if absent, can decrease the current total P of lung cancer, which is 0.402, as shown in the last cell of the corresponding mini-max table (see table on page 34.) The P of lung cancer before considering clinical data absent was 0.444 (equals greatest PP value in the second column, corresponding to dyspnea present); S of pulmonary mass on X-ray films for lung cancer is 0.9, as shown in the corresponding S LIST.

To calculate resultant P that a best cost-benefit clinical datum assumed absent confers to each respective diagnosis, we calculate new partial P only for one cell in the new column of corresponding mini-max table; but as we must achieve this for all diagnoses in the differential diagnosis list, we apply equation 8 to each of these diagnoses in the clinical data pair Dyspnea-Mass:

| Dyspnea-Mass | PP value | S | | | Partial P |
|---|---|---|---|---|---|
| Pulmonary | 0.148 × | (1 − 0.10) = | 0.133 | ÷ 0.566 = | 0.235 |
| tuberculosis | | | | | |
| Pulmonary | 0.370 × | (1 − 0.05) = | 0.352 | ÷ 0.566 = | 0.622 |
| embolism | | | | | |
| Bronchiectasis | 0.037 × | (1 − 0.00) = | 0.037 | ÷ 0.566 = | 0.065 |
| Lung cancer | 0.444 × | (1 − 0.90) = | 0.044 | ÷ 0.566 = | 0.078 |
| | | | 0.566 | | 1.000 |

For this example we show equation 8 applied only to the diagnosis lung cancer and the corresponding mini-max table:

$${P}_{lumg cancer} == \frac{PP {value}_{lung caner} ( 1 - {S}_{lung cancer} )}{\begin{matrix}{PPvalue}_{lung cancer} ( 1 - {S}_{lung cancer} ) + \\ {PPvalue}_{TB} ( 1 - {S}_{TB} ) + \\ {PPvalue}_{embolism} ( 1 - {S}_{embolism} ) + \\ {PPvalue}_{bronchiectasis} ( 1 - {S}_{bronchiectasis} )\end{matrix}}$$

$$\begin{matrix}{P}_{lung cancer} = \frac{0.444 ( 1 - 0.90 )}{\begin{matrix}0.444 ( 1 - 0.90 ) + 0.148 ( 1 - 0.10 ) + \\ 0.370 ( 1 - 0.05 ) + 0.037 ( 1 - 0.00 )\end{matrix}} \\ = \frac{0.044}{0.044 + 0.133 + 0.352 + 0.037} \\ = 0.078\end{matrix}$$

Mini-Max Table for Lung Cancer when Pulmonary Mass is Absent

| | PP value = partial | | | | |
|---|---|---|---|---|---|
| | P before | | | | |
| | considering | | | | MINIMUM |
| | absent clinical | Cavity absent | Fever absent | Mass absent | VALUE |
| LUNG CANCER | data | S = 0.3 | S = 0.1 | S = 0.9 | IN EACH ROW |
| Cough | 0.241 | 0.231 | 0.297 | | |
| present | | | | | |
| Hemoptysis | 0.278 | 0.254 | 0.349 | | |
| present | | | | | |
| Dyspnea | 0.444 | 0.402 | 0.540 | 0.078 | 0.078 |
| present | | | | | |
| Expectoration | 0.104 | 0.109 | 0.135 | | |
| present | | | | | |
| MTb | 0.000 | 0.000 | 0.000 | | |
| present | | | | | |
| MAXIMUM VALUE | 0.444 | 0.402 | 0.540 | | Total P = 0.078 |
| IN EACH COLUMN | | | | | |

MTb, Mycobacterium tuberculosis

According to the above explanation, as expected total P (0.078) is smaller than the current P (0.402), “Mass absent” is recommended as best cost-benefit clinical datum. If this datum is confirmed absent, entered in the computer, and processed, the complete mini-max procedure is applied and all cells of the mini-max tables, including the new column, will be filled with partial P.

### Mini-Max Table for Lung Cancer

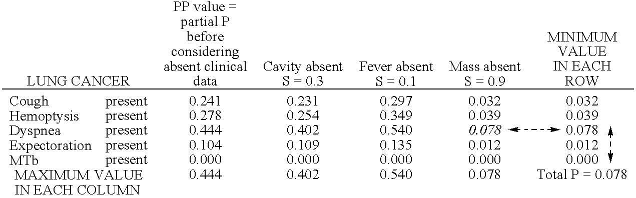

MTb, Mycobacterium tuberculosis
Mini-Max Table after Mass absent confirmed absent

All diagnoses in each cost category are similarly processed. The user is prompted each time the loop goes to a greater cost category. The remaining differential diagnoses with their P are displayed and the user is asked whether he wants to proceed in the greater cost category or prefers a deferred diagnosis, diagnosis by exclusion, or empirical treatment. The entire looping process terminates when all final diagnoses are obtained, competing diagnoses are ruled out, the cost of investigating recommended clinical data exceeds the benefit, or all the clinical data able to change P of diagnoses are processed. Clinical data that have the greatest PP value or the greatest S typically involve costly pathological investigations, such as biopsy or even autopsy. To request a biopsy or even an autopsy for a patient with tonsillitis would make little sense. This exaggeration emphasizes the importance of initially considering the cost of a clinical datum, before evaluating its PP value or S. However, in an emergency or when a patient's condition is deteriorating, investigation of confirmatory clinical data of great PP value takes priority over cost.

In summary, to select the best cost-benefit clinical datum to investigate next, the algorithm loops at three nested levels (Diagram page 40): outer, intermediate, and inner. (1) The outer cost loop processes clinical data not yet investigated in order of increasing cost category: none, small, intermediate, and great. (2) Within each cost category, the intermediate diagnosis loop processes the diagnoses of the differential diagnosis list in order of decreasing P because those with greatest P values, are the best candidates for a final diagnosis, and can sooner conclude the diagnostic quest. (3) The inner clinical data loop comprises two sub-loops: the first begins at the top of the PP value list and terminates when no clinical datum exists able to increase current P of diagnoses. The second sub-loop begins at the top of the S list and terminates when no clinical datum exists able to decrease current P of diagnoses.

Each new best cost-benefit clinical datum present creates a new clinical datum list that includes the diagnosis from which it was selected. This diagnosis appears in some or all previous clinical datum lists because it originated the search for the new clinical datum; this datum just increases the number of clinical data that support this diagnosis. Some of the new clinical datum lists may include previously unlisted diagnoses that also may manifest this clinical datum. When this occurs, such new diagnoses will not have clinical data in common with any previous diagnosis because they were not included in previous clinical datum lists; accordingly, previous and new diagnoses must be concurrent. New clinical datum lists bring up new diagnoses; these, in turn, bring up new clinical data. At first thought, this cycle may seem to iterate indefinitely until the universe of clinical data is exhausted. In reality, this does not occur, because a single patient cannot manifest all clinical data. At some point, the newly recommended best cost-benefit clinical datum will simply be absent and will not create a new clinical datum list, aborting the cycle; still, it must be investigated so as to confirm its absence. Neither can a patient be afflicted by a multitude of concurrent diseases. Another factor limiting the number of diagnoses is that a best cost-benefit clinical datum is selected for its great PP value and accordingly is either pathognomonic for a single diagnosis or supportive of only a few diagnoses.

The recommended best cost-benefit clinical datum could be a common symptom quickly asked or immediately observed by the physician. Should obtaining a clinical datum require an involved test or procedure, the diagnostic process must be interrupted until the result becomes available. The “position of the game board”, so to say, must be saved in the computer and opportunely retrieved to continue the “game”, because each new clinical datum, with its presence or absence in the patient, sets a new stage for the selection of the next best cost-benefit clinical datum. A disease is not a static process; if the clinical picture changes considerably prior to obtaining the diagnostic procedure result, a new diagnostic evaluation must be accomplished, sometimes from the beginning.

In our previous main patent application and also in this continuation-in-part, we stated that a best cost-benefit clinical datum selected from the recommended data present list (PP value list) but investigated in the patient is actually absent, should be disregarded. Similarly, a best cost-benefit clinical datum selected from the recommended data absent list (S list) but investigated in the patient is actually present, should be disregarded. However we decided to enter anyway those previously disregarded clinical data in the computer, because, although they will not change diagnosis P, they will be a remainder for the user that those clinical data were already processed, and will not be recommended again.

### Simultaneous Recommendation of Best Cost-Benefit Clinical Data

Simultaneous recommendation of best cost-benefit clinical data is essential for a diagnostic algorithm to be able to diagnose actual patient cases. Two examples, one dramatic, and the other non-dramatic will illustrate this statement. (1) Dramatic example: a patient is rushed by ambulance to an emergency service; he is suffering an acute myocardial infarction complicated with congestive heart failure, cardiac arrhythmia, and shock. A program that recommends best cost-benefit clinical data sequentially, would first recommend an electrocardiogram; twenty minutes later, when the report is entered in the computer, the next recommendation would be a troponin analysis, then chest x-rays, and so on.

Four hours later, the necessary tests to complete the diagnostic quest are finally available; unfortunately the patient expired in the meantime. (2) Non-dramatic example: a patient comes to your office with fever, cough, and some abdominal discomfort. The computer recommends one test, perhaps a complete blood count; after two days the patient returns to your office for the result. This time the computer recommends an erythrocyte sedimentation rate, the patient has to have blood drawn again, return after another two days, and the physician still does not know what is going on. It is most likely that after the third visit the patient is consulting another physician who has no computer.

Few diagnostic computer programs recommend, based on probability calculation, a single best cost-benefit clinical datum next to investigate; we know of none that simultaneously recommends a set of such data.

Physicians typically order a set of several analyses, tests, or procedures simultaneously; A computer can emulate such human behavior by iterating the best cost-benefit clinical data function, first assuming each newly recommended best cost-benefit clinical datum as virtually present and then as virtually absent, while observing the effect that each iteration has on the P of each diagnosis.

Such iteration can be represented as a trichotomy tree (see diagram on next page). Each tree represents a single diagnosis in the differential diagnosis list. Virtual branches represent best cost-benefit clinical data present or absent; nodes represent the P and cost of the diagnosis. Each node originates three new branches; four cost level iterations are involved; accordingly, the total number of branches is 3¹+3²+3³+3⁴=120. Each top branch originating at a node assumes that the best cost-benefit clinical datum-selected from the PP value list—is present; accordingly, it increases P of the diagnosis and is depicted by an ascending arrow. Each middle branch assumes that this best cost-benefit clinical datum is absent; accordingly, it is disregarded, does not change P, and is depicted by a horizontal arrow. Each bottom branch assumes that the best cost-benefit clinical datum—elected from the S list—is absent; accordingly, it decreases P and is represented by a descending arrow. The same middle branch also assumes that this best cost-benefit clinical datum, selected from the S list, is present, disregarded, and does not change P. Additionally, this middle branch represents situations in which no best cost-benefit clinical datum was found in either the PP value list or S list; accordingly, it offers no best cost-benefit clinical data to investigate. This reduces the best cost-benefit clinical data to investigate to only two branches per node, the top (present) and the bottom (absent) branches; the middle branch is preserved however, because it takes us to a next node. In the entire tree, the total number of best cost-benefit clinical data to investigate now is reduced to 80. This result, multiplied by the number of diagnoses in the differential diagnosis list yields the number of best cost-benefit clinical data to investigate, provided no best cost-benefit clinical data are shared with other trees (diagnoses).

A clinical datum frequently is shared by diverse diagnoses. However, a best cost-benefit clinical datum selected from the PP value list has a great PP value; and when present, it is either very specific or pathognomonic for a diagnosis. Such clinical datum is typically not shared with any other tree. When a best cost-benefit clinical datum is selected from the S list and is absent, it might be shared by diverse diagnoses, for which this clinical datum occurs frequently.

In a single tree, the best cost-benefit clinical datum present, represented by the top branch, typically has a great PP value and strongly supports a diagnosis. The best cost-benefit clinical datum absent, represented by the bottom branch, typically has a great S and strongly opposes a diagnosis. Occasionally, an identical best cost-benefit clinical datum, when it has a great PP value and a great S, may be recommended simultaneously in the top and bottom branch. This apparent opposition is not conflicting because it refers to virtual present and absent alternatives that do not coexist in a real patient case.

Processing the entire set of best cost-benefit clinical data perhaps could at once confirm as final those diagnoses with a P close to 1 and rule out those with a P close to 0. However, exhaustively traversing all branches of this exponentially growing trees is limited by the increasing number of clinical data to investigate and the cost involved. At best, such an approach will enable us to move only a few steps forward. Fortunately, heuristic shortcuts might dispel this concern. As noted on page 38, clinical data present of great PP value that strongly favor a diagnosis are unlikely to be opposed by clinical data absent of great S that strongly disfavor the same diagnosis. Accordingly, when a diagnosis with an initial great P is processed, we would expect the algorithm to recommend a best cost-benefit clinical datum of greater PP value that would further enhance that P, rather than recommend a best cost-benefit clinical datum of great S. Conversely, when a diagnosis with an initial small P is processed, we would expect the algorithm to recommend a best cost-benefit clinical datum of greater S that would further reduce that P, rather than recommend a best cost-benefit clinical datum of great PP value. This expectation would favor virtual traversing from present to present branches toward greater P values of the diagnosis or from absent to absent branches toward smaller P values of the diagnosis. Thus in the tree, the process would tend to traverse solid exterior branches only, while avoiding zigzag traversal along dashed alternating present and absent interior branches.

If we elect not to exclusively traverse extreme exterior branches, a few virtual best cost-benefit clinical data alternatively absent and present can be accepted. This maintains traversal near the exterior branches, leading to nodes with P near to diagnosis confirmation or elimination values.

Ideally, the algorithm explores—for each diagnosis in the differential diagnosis list—all possible virtual traversals until maximal or minimal P are attained, or until all available clinical data are exhausted. To accomplish this goal, prompts and authorization requests to continue in the next greater cost category must be bypassed. Cost momentarily is disregarded so as to obtain an ample overview of all best cost-benefit clinical data available. A decision regarding which best cost-benefit clinical data to select and up to what cost can be made afterwards according to disease severity.

Experimenting with our new prototype program showed that recommended best cost-benefit clinical data could be very numerous, sometimes several hundreds, increasing prohibitively the cost and burden to investigate them. This mandated the devising of heuristic strategies to reduce this number without compromising the efficiency and accuracy of the diagnostic quest.

Regarding which heuristic strategies to devise for selecting a set of recommended best cost-benefit clinical data, the following considerations seem valid. An important advantage of our program is that it lists all best cost-benefit clinical data able to modify P of each diagnosis, and each clinical datum recommended shows in advance the value of resulting P when this clinical datum is found either present or absent. A tradeoff exists between the burden or cost of requesting an excessive number of recommended best cost-benefit clinical data and the risk of missing valid diagnoses by requesting an insufficient number. The following strategies and parameters are devised to reduce the number of best cost-benefit clinical data recommended while minimizing the risk of missing valid diagnoses.

Our program involves four types of heuristic restricting strategies to safely reduce the number of best cost-benefit clinical data to investigate: (1) Each clinical datum that yields more benefit (greater diagnosis P change) supersedes and removes all other clinical data in the same cost category that produce less benefit (smaller diagnosis P change). (2) Grouping clinical data according to test or procedure necessary to investigate them. (3) Parameters. (4)

Abridged best cost-benefit files. These strategies are not mutually exclusive; they are applied simultaneously.

### 1. Best Cost-Benefit Clinical Data Superseding and Removing Less Beneficial Clinical Data

While our new program first displays all best cost-benefit clinical data, as soon as such a clinical datum is confirmed present or absent, all other clinical data that produce a smaller change of P in the same cost category and for the same diagnosis are removed from the recommended clinical data list, which reduces remarkably the number of clinical data to investigate in the patient.

2. Grouping Clinical Data According to Test or Procedure Necessary to Investigate them

The total amount of best cost-benefit clinical data recommended simultaneously may be large, but many of these diverse clinical data can be investigated by a single procedure (e.g., a colonoscopy investigates at a single session intestinal polyps, diverticula, ulcers, masses, etc.; a single blood draw provides a sample used to investigate multiple clinical data, such as complete blood count, erythrocyte sedimentation rate, chemistry, and others). Typically, an apparently great number of clinical data recommended can be investigated with only a few diverse procedures (e.g., laboratory tests, ECG, and chest X-rays). We realized the importance of the diagnostic program to display the recommended best cost-benefit clinical data grouped by procedures necessary to investigate and order them. In a modern, well organized medical environment these orders could be automatically transmitted, via email, to the corresponding facility: clinical laboratory, radiology, procedure specialist, suggesting at the same time the clinical data most important to confirm present or absent. What actually matters is the number of diverse types of tests involved; frequently, a great number of data can be investigated simultaneously with only a single blood draw, urine sample, imaging, or procedure. However, the cost of diverse tests or procedures may be additive. Our program displays recommended best cost-benefit clinical data sorted by cost and type of test or procedure in output files Data Cost Procedure Quantity, Abridged Data Cost Procedure Quantity, Global Overview, and Abridged Global Overview files, which facilitates requesting them simultaneously (these files will be explained later).

### 3. Parameter Strategies

An input file called Parameters enables the user to select empirical values for these parameters, which limit the number of best cost-benefit clinical data to investigate.

### Trim No Cost Parameters

Trim Present No Cost parameter removes only from no cost best cost-benefit clinical data present those recommended clinical data present unable to increase P of diagnosis more than the parameter empirical value, leaving those clinical data able to produce a change of P equal or greater than the value at which the parameter was set. We exempted from being affected and removed by this strategy only the clinical datum present that results in the greatest increase of P (upper exterior arrow of trichotomy tree), because we consider it of great diagnostic importance. The greater the Trim Present value is set, the less recommended best cost-benefit clinical data are displayed, but the more inaccurate the diagnostic quest may become. However, the clinical data removed are the ones at the bottom of the no cost best cost-benefit clinical data present list (PP value list) which produce little change in P of the diagnosis and will most likely be superseded by best cost-benefit clinical data producing a greater P change. Small values of this parameter and consequent small changes of diagnostic P do not affect diagnostic accuracy; we tentatively set this parameter at 100 (0.10). However, the need and magnitude of such empirical approach will be better evaluated when a database with all known diseases and clinical data will become available.

Trim Absent No Cost parameter removes only from no cost best cost-benefit clinical data absent those recommended clinical data absent unable to decrease P of diagnosis more than the parameter empirical value, leaving those clinical data able to produce a change of P equal or greater than the value at which the parameter was set. We exempted from being affected and removed by this strategy only the clinical datum absent that results in the greatest decrease of P (lower exterior arrow of trichotomy tree), because we consider it of great diagnostic importance. The greater the Trim Absent value is set, the less recommended best cost-benefit clinical data are displayed, but the more inaccurate the diagnostic quest may become. However, the clinical data removed are the ones at the bottom of the no cost best cost-benefit clinical data absent list (S list) which produce little change in P of the diagnosis and will most likely be superseded by best cost-benefit clinical data producing a greater P change. Small values of this parameter and consequent small changes of diagnostic P do not affect diagnostic accuracy; we tentatively set this parameter at 100 (0.10). However, the need and magnitude of such empirical approach will be better evaluated when a database with all known diseases and clinical data will become available.

The no cost best cost-benefit clinical data display compensates for an incomplete medical history and physical examination, prompting the examiner to do a better job. Investigating more initial no cost clinical data present during the first consultation evokes more diagnoses, brings P of diagnoses closer to their final values, which in turn reduces the number of best cost-benefit clinical data to be displayed in greater cost categories, and reduces the possibility of missing concurrent diagnoses, were the newly processed clinical data to introduce new potential diagnoses.

### Trim Greater Cost Parameters

When no cost clinical data do not conclude the diagnostic quest, best cost-benefit clinical data are selected from small and intermediate cost categories. The reason why we created a separate trim parameter of greater cost clinical data is because in this case, tests or procedures are required and presence or absence of selected clinical data cannot be immediately verified. Therefore, to preclude investigating the entire list of best cost-benefit clinical data recommended in these cost categories, alternative strategies are applied to select the most convenient ones.

Trim Present Greater Cost parameter deserves exactly the same comments as Trim Present No Cost (see above), with the only difference that it applies to small, intermediate, and great cost best cost-benefit clinical data present instead of no cost clinical data.

Trim Absent Greater Cost parameter deserves exactly the same comments as Trim Absent No Cost (see above), with the only difference that it applies to small, intermediate, and great cost best cost-benefit clinical data absent instead of no cost clinical data.

When the best cost-benefit clinical datum in small cost category yields little P change of the corresponding diagnosis, whereas the one in intermediate cost category yields a much greater P change, skip the former cost category and directly select the datum from the latter. Leave great cost clinical data to subsequent diagnostic rounds. Conversely, if a smaller cost category datum yields confirmation or deletion thresholds for the diagnosis, obviously greater cost category data will be disregarded.

### Difference Cost Parameters

Present Difference Cost compares P result of best cost-benefit clinical data present in great cost category with P results achieved by best cost-benefit clinical data in lower cost categories (no, small, and intermediate cost) and removes from great cost best cost-benefit clinical data present those recommended clinical data unable to increase P of diagnosis an additional value, equal or greater than the value set for this parameter. This precludes the selection of great cost clinical data when they produce no or only insignificant extra increase of P. If the selected smaller cost clinical datum happens to be absent, the removed great cost data will be redisplayed at next program iteration. When a smaller cost clinical datum results in the same P than a great cost clinical datum, setting the parameter at an even very small Present Difference Cost value, such as 001, is sufficient to remove the clinical datum in the great cost list.

Absent Difference Cost compares P result of best cost-benefit clinical data absent in great cost category with P results achieved by best cost-benefit clinical data in lower cost categories (no, small, and intermediate cost) and removes from great cost best cost-benefit clinical data absent those recommended clinical data unable to decrease P of diagnosis an additional value, set with this parameter. This precludes the selection of great cost clinical data when they produce no or only insignificant extra decrease of P. If the smaller cost clinical data happens to be present, the removed great cost data will be redisplayed at next program iteration. When a smaller cost clinical datum results in the same P than a great cost clinical datum, setting the parameter at an even very small Absent Difference Cost value, such as 001, is sufficient to remove the clinical datum in the great cost list.

When any diagnosis in the differential diagnosis list has not been confirmed or ruled out, especially when patient's condition is urgent or critical, select at once great cost best cost-benefit clinical data, unless cost is prohibitive in the context of medical-social circumstances.

### Confirmation Threshold and Deletion Threshold Parameters

Confirmation threshold for diagnoses enables the user to select an empirical P value for this parameter. Diagnoses that reach P equal or greater than this parameter are confirmed as final diagnoses. Our current tentative default level is P=900 (0.90).

Deletion threshold for diagnoses enables the user to select an empirical P value for this parameter. Diagnoses that reach P equal or smaller than this parameter are ruled out. Our current tentative default level is P=100 (0.10).

Confirmation and deletion thresholds reduce the number of best cost-benefit clinical data to investigate when judiciously setting their values. The lower the former and the higher the latter, the less clinical data are necessary to reach their levels and the less diagnoses remain to be processed, but also the greater the risk to improperly confirm or rule out diagnoses.

However, an advantage of our current diagnostic program is that diagnoses ruled out are not definitively deleted; when entering any new clinical datum, present or absent, our algorithm iterates from the beginning, reprocessing all steps. If some new clinical data increase the P of the ruled out diagnoses above the deletion threshold, the corresponding best cost-benefit clinical data will be redisplayed.

### Cutoff Parameters

Cutoff Present enables the user to select a P cutoff level that removes all recommended best cost-benefit clinical data from the data present list (considered not worth to be investigated) for diagnoses with P below this level. This cutoff point should be set at a level that reasonably separates the clinical data below, very unlikely to result in a P able to confirm the diagnosis, from the clinical data above that have the potential, in next iterations, to increase P to a confirmatory value. Our current tentative default level is P=200 (0.20). Cutoff Present level represents the lower limit that together with the confirmation threshold, representing the upper limit, define a zone or range that encompasses the best cost-benefit clinical data present recommended to be processed (see diagram in next page). Cutoff Present strategy implies the risk of ruling out potentially correct diagnoses with their P temporarily below this level. This is not critical because the removed diagnoses are not definitively deleted; they remain hidden and will be reprocessed at every new program iteration, and corresponding best cost-benefit clinical data will be redisplayed if some new supporting clinical data increase diagnoses P above the Cutoff Present level.

Cutoff Absent enables the user to select a P cutoff level that removes all recommended best cost-benefit clinical data from the data absent list (considered not worth to be investigated) for a diagnosis with a P above this level. This cut off point should be set at a level that reasonably separates the clinical data above, very unlikely to result in a P able to delete the diagnosis, from the clinical data below that have the potential, in next iterations, to decrease P to a rule out value. Our current tentative default level is P=500 (0.50). Cutoff Absent level represents the upper limit that together with the deletion threshold, representing the lower limit, define a zone or range that encompasses the best cost-benefit clinical data absent recommended to be processed. Both zones limited by cutoff present and cutoff absent tend to overlap partially. Cutoff Absent strategy implies the risk of ruling out potentially correct diagnoses with their P temporarily above this level. This is not critical because the removed diagnoses are not definitively deleted; they remain hidden and will be reprocessed at every new program iteration, and corresponding best cost-benefit clinical data will be redisplayed if some new clinical data decrease the diagnoses P below the Cutoff Absent level.

### 4. Abridged Output Files

This strategy to reduce the number of best cost-benefit clinical data necessary to investigate involves abridged output files, discussed in the next section while describing our diagnostic program.

### Programs Running on Microsoft Windows

Our computer diagnostic programs are written in C language, compiled with Metrowerks CodeWarrior, and open in Microsoft Windows.

Our diagnostic system comprises a Diagnostic Program, a Data Program, Input Files, and Output Files, as summarized in the table on pages 61 through 65. Programs impart instructions to the computer. Input files provide information to the computer; this information is stored in the database. Output files retrieve information from the computer.

### Programs

Diagnostic Program. Double clicking its shortcut icon on the desktop opens a window titled iomed.mcp. Pressing F5 key runs the program; a black background window shows “process terminated”. Iomed.mcp window and black window can now be closed. Each time a change is made in any of the input files, this program must be run again, to update output files.

Datum Program is an auxiliary program that creates clinical datum lists by reciprocating the information of Disease Models; instead of displaying for each disease model the corresponding clinical data, it displays for each clinical datum all the diagnoses able to manifest it. Clinical Datum Lists is the name of the created output file. After any change in Disease Models this program must be run to update Clinical Datum Lists.

### Index Files

Double clicking its shortcut icon on the desktop opens a window titled Index Files; it lists in alphabetical order all input and output files, differential diagnosis list with diagnoses sorted by decreasing P values, complex clinical presentation models, and interacting (masking) diagnoses. Index Files lists also some individual patient files with clinical data present and clinical data absent as examples to challenge the performing of our program. These examples have as title the name of the disease case from where they were obtained. This title is only for user's information; the program ignores these titles, and in actual cases the user does not know the diagnosis at the time he enters the clinical data in the computer. Double clicking any item in the Index Files opens the corresponding file.

All differential diagnoses supported by at least one clinical datum present are shown in Index Files with the format Rxxxx-xxx-Dxxxx-<DIAGNOSIS>. Rxxxx shows the ranking place this diagnosis occupies in the entire differential diagnosis list, from greatest (ordinal R0001) to the smallest (greatest ordinal Rxxxx). A three digit number (xxx) follows, which represents the P of the diagnosis expressed in per mill as an entire number (as opposed to decimal), sorted by decreasing P; because our current programs allows only these three digits, KKK represents a P=1000 per thousand (1.000). Dxxxx is an arbitrary number that identifies each diagnosis in the data base, followed by the name of this diagnosis. The entire differential diagnosis list is displayed in Index Files, including even diagnoses with P=0 as long as they have at least one supporting clinical datum. Once a deletion threshold for ruling out diagnoses is selected, it is easy to visualize the number of diagnoses remaining to be processed, equal to the ranking number that follows R of the last diagnosis, which P is equal or greater than this threshold. Double clicking any of these diagnoses opens a window showing the corresponding diagnoses with its title Rxxxx-xxx-Dxxxx-<DIAGNOSIS>. This title is followed by the PP value of the clinical datum that most favors this diagnosis and S of the clinical datum that most disfavors it (if clinical data absent were entered). Then, the determining clinical data pair in the mini-max table (not necessarily the same data previously mentioned), and the total P of this diagnosis in the differential diagnosis list are displayed. All this is followed by the recommended best cost-benefit clinical data assumed present with respective PP values and resultant P, and recommended best cost-benefit clinical data assumed absent with respective S values and resultant P. These clinical data are grouped by increasing cost categories and sorted by resulting P values. The format of each opened diagnosis is identical to the format of the same diagnosis in Comprehensive Differential Diagnosis List output file, the latter listing also all the other diagnoses in the entire differential diagnosis list (see below).

Complex clinical presentation models are listed with the format Lxxxx-Rxxxx-Dxxxx-<DIAGNOSIS>. L is followed by a four digit number (Lxxxx) identifying the model to which the diagnosis belongs (same number for each diagnoses belonging to same model) and these models are sorted by their decreasing P (each equal to the greatest P of the diagnoses in this model). R is followed by a four digit number (Rxxxx) that shows again the ranking place this diagnosis occupies in the entire differential diagnosis list, according to its decreasing P; R followed by capitalized RXXXX represents a diagnosis that belongs to the model but is not in the differential diagnosis list because so far it has no supporting clinical data present. D is followed by a four digit number (Dxxxx) which is the mentioned arbitrary number that identifies each diagnosis in the database, followed by the name of this diagnosis. When any of these diagnoses is opened, the window's title shows the complex disease model to which it belongs (name of the model and its arbitrary number, preceded by the letter M—for Mode—if it is a model of related diagnoses, or I—for Interactions—if it is a model of masking diagnoses). Next to the model title, the file repeats the diagnosis information Lxxxx-Rxxxx-Dxxxx-<DIAGNOSIS>; the remaining format is identical to the one described for R diagnoses. The format of each opened diagnosis is identical to the format of the same diagnosis in the Complex Comprehensive output file together with all the other diagnoses in the same model, and identical to Comprehensive Differential Diagnosis List output file together with all the other diagnoses in the entire differential diagnosis list (see below).

### Input Files

Disease Models list number and name of all diagnoses (Dxxxx <DIAGNOSIS>), each listing all clinical data that the corresponding disease can potentially manifest, grouped by cost categories. Here, for technical reasons, cost categories—no, small, intermediate, and great costs—are represented by E1, E2, E3, and E4 respectively. Each clinical datum is followed by its sensitivity (S0.00 . . . S1.00); PP values are not shown in this file, but can be found in the output file Clinical Datum Lists. Any disease model can be found in the input file Disease Models when searched with Find function.

Present Data is the input file for clinical data present in the patient.

Absent Data is the input file for clinical data absent in the patient.

Complex Presentation Models lists number and name of complex clinical presentation models (Mxxxx <MODEL NAME> or Ixxxx <MODEL NAME>), and number and name of the diagnoses (Dxxxx <DIAGNOSIS>) comprised by each model.

Data Procedures lists all known clinical data (Cxxxx <NAME OF CLINICAL DATUM>) grouped by procedure or test to obtain them (Gxxxx <NAME OF PROCEDURE>).

Parameters can be set to diverse empirical values to reduce the number of clinical data necessary to investigate in the patient, simplifying and shortening the diagnostic process. These 10 parameters, explained earlier and summarized in table pages 61 through 65, are: Trim Present No Cost, Trim Absent No Cost, Trim Present Greater Cost, Trim Absent Greater Cost, Present Difference Cost, Absent Difference Cost, Cutoff Present, Cutoff Absent, Confirmation Threshold, and Deletion Threshold.

### Output Files

Comprehensive Differential Diagnosis List displays all the differential diagnoses, sorted by decreasing P. Each diagnosis is separated from the next with a dashed line (----------) allowing fast navigation from one to the other with Find Next function (Find what: ----------). It shows the arbitrary number and name of each diagnosis (Dxxxx <DIAGNOSIS>) followed by the number and name of the clinical datum present with greatest PP value (Cxxxx <CLINICA DATUM> <PP value>)—most favoring it—and the number and name of the clinical datum absent with the greatest S—most disfavoring it (Cxxxx <CLINICA DATUM> <S>). Next, follows mini-max result showing the determining clinical data pair and the resulting total P. Notice that a few times, the greatest PP value is different from the clinical datum present in the determining clinical data pair, although this is more frequent with the clinical datum absent with greatest S being different from the clinical datum absent in the determining clinical data pair. This difference—irrelevantly small—when it occurs, is indicative of broken monotony (page 35) among clinical data processed at pervious program iterations. Sorted by increasing cost categories, a complete and typically quite long list of best cost-benefit clinical data present and absent able to change P of this diagnosis follows. For each best cost-benefit clinical datum the resultant P change, were this datum to be processed, is indicated, sorted by decreasing magnitude; for each datum present and for each datum absent, the respective PP value or S is also shown. For best cost-benefit clinical data assumed present, ordered by decreasing P, a rare broken monotony is detected when the expected parallel decreasing of respective PP values is disrupted. Similarly, for best cost-benefit clinical data assumed absent, ordered by increasing P, a more frequent broken monotony is detected when the expected opposite decreasing of respective S is disrupted. This broken monotony does not significantly affect the accuracy of diagnostic results, because the consequent difference in P values is small and because best cost-benefit clinical data are selected based on their resultant P, correctly sequenced and not by their disrupted PP values or S sequences.

Differential Diagnosis List is a partial display of Comprehensive Differential Diagnosis List. It displays all differential diagnoses sorted by descending P calculated with mini-max procedure, showing the determining clinical data pair. This file is not affected by Parameters and the differential diagnoses list is not abridged.

Global Overview recommends the best cost-benefit clinical data after parameter settings. It includes data that check for related diagnoses in complex clinical presentation models and potentially masking diagnoses and drugs. The best cost-benefit clinical data are grouped by cost, displaying their quantity in each category and total number recommended. In each cost category, the data are sub grouped by procedure to obtain them, diagnosis to which each refer, presence or absence of the datum to produce P change, and showing for each affected diagnosis its current P and the resulting P after processing the recommended datum. This file provides the most useful information the user needs to select a set of best cost-benefit clinical data.

Abridged Global Overview is an abridged version of Global Overview with similar grouping, but displaying only the best cost-benefit clinical datum with greatest PP value and S in each cost category (clinical data represented by the exterior arrows of the trichotomy tree, page 48), able to produce P change of corresponding diagnosis, after parameter settings. Because there are four cost categories. (no, small, intermediate, and great cost), and for each of them, 2 exterior arrows (clinical datum present with greatest PP value and clinical datum absent with greatest S), at most only 8 clinical data are recommended per diagnosis, but if some are not able to change P then these data will be even less. Abridged Global Overview achieves an important heuristic reduction in the number of recommended best cost-benefit clinical data, with program remaining highly efficient and accurate.

Data Cost Quantity recommends the best cost-benefit clinical data able to change P of diagnoses after parameter settings, grouped by cost categories and indicating partial quantity of these data in each, and total quantity. The limitations of Data Cost Quantity is that it does not indicate the procedure to obtain these data, diagnosis to which each refer, recommendation for presence or absence, nor resultant P after processing; if this information is sought, the user must resort to other files. However, if there is no need for this missing information, the advantage of this file is that the recommended best cost-benefit clinical data are not so dispersed as in other files, but in more compact groups. When they are numerous, it is easier and faster to copy them in blocks and paste them side by side from Data Cost Quantity into Present Data and Absent Data files, avoiding the need to “Find” them one by one.

Abridged Data Cost Quantity is an abridge version of Data Cost Quantity, displaying only the best cost-benefit clinical data with greatest PP value and S in each cost category, able to produce P change after parameter settings, indicating quantity of data in each cost category and total quantity. Same as Data Cost Quantity it facilitates coping and pasting of clinical data into Present Data and Absent Data files.

Data Cost Procedure Quantity recommends best cost-benefit clinical data able to change P of diagnoses after parameter settings, grouped by cost categories, displaying quantity in each category, sub grouped by procedure to obtain them. This file is similar to Data Cost Quantity, but adds procedure to obtain each clinical datum. There is no mention of diagnoses, presence or absence of clinical data, nor P. Same as Data Cost Quantity it facilitates coping and pasting of clinical data into Present Data and Absent Data files.

Abridged Data Cost Procedure Quantity is an abridge version of Data Cost Procedure Quantity, displaying only the best cost-benefit clinical data with greatest PP value and S in each cost category, able to produce P change after parameter settings, indicating quantity of data in each cost category, total quantity, and procedure to obtain them. Same as Data Cost Quantity it facilitates coping and pasting of clinical data into Present Data and Absent Data files.

Parameter Affected Differential is a differential diagnosis list with all diagnoses sorted by descending P, but displaying best cost benefit clinical data only for diagnoses with P above deletion threshold, after parameter settings.

Abridged Parameter Affected Differential is an abridged version of Parameter Affected Differential, displaying only the best cost-benefit clinical data with greatest PP value and S in each cost category, able to produce P change after parameters setting.

Clinical Datum Lists displays every clinical datum in the database with its arbitrary number and its cost; then, it lists all diagnoses that can manifests it, showing S and PP value of this datum for each listed diagnosis. It is the reciprocal of Disease Models, which list every disease in the database with all the clinical data potentially able to manifest.

Numbered Data lists all clinical data in the database sorted by their identifying numbers assigned arbitrarily.

Complex Comprehensive displays all complex clinical presentation models with corresponding name and arbitrary number preceded by M (for Model) if listing related diagnoses, and 1 (for Interaction) if listing potentially masked and masking diagnoses or drugs. Disease models are assigned a P equal to the greatest P of the diagnoses in the model and they are sorted by decreasing P. Diseases inside each model are also sorted by decreasing P, each listing all best cost-benefit clinical data able to change diagnoses P, sorted by cost category and whether present or absent, showing resultant P, were these clinical data to be processed. The format of each individual diagnosis in the complex clinical presentation models is identical to the display for the same diagnosis in L and R on Index Files, and in Comprehensive Diagnostic List. Some diagnoses neither show P nor mini-max result; this occurs when no supporting clinical data present exist for these diagnoses.

Complex Short is an abridged version of Complex Comprehensive, displaying related diagnoses and masking diagnoses and drugs, displaying only respective P for diagnoses supported by clinical data present, without further information. Diagnoses not supported by any clinical datum are listed, but without any further information.

The table on next pages, summarizes programs, input files, and output files with corresponding descriptions.

### Strategies Depending on Abridged Output Files

We call the resulting reduced output files after applying this strategy Abridged files, preceding the corresponding non abridged file name with this term: Abridged Data Cost Quantity, Abridged Data Cost Procedure Quantity, Abridged Parameter Affected Differential, and Abridged Global Overview.

| FILE TYPE | NAME | | DESCRIPTION |
|---|---|---|---|
| PROGRAMS | Diagnostic Program | | After a change in any INPUT FILES, |
| To ran these | | | running this program updates all output |
| programs, click | | | files except Clinical Datum Lists |
| corresponding | Datum Program | | After any change in the database this |
| shortcut on | | | program must be run to update |
| desktop and | | | Clinical Datum Lists |
| press F5 key | | | |
| FILE | Index Files | | Lists all input and output files; each |
| CONTENTS | | | opens when corresponding icon in |
| | | | NAME column is clicked |
| INPUT | | | Lists number and name of diagnoses (D), |
| FILES | | | cost category of clinical data (E), number |
| (continues) | | | and name of clinical data (C) with their |
| | | | sensitivities (S) for the corresponding |
| | | | diagnosis |
| | Present Data | | Clinical data present |
| | Absent Data | | Clinical data absent |
| | Complex Presentation Models | | Complex clinical presentation models (M |
| | | | or I), listing related diagnoses, masking |
| | | | diagnoses, and drugs (D) |
| | Data Procedures | | All clinical data grouped by procedure or |
| | | | test to obtain them |
| | Parameters | Trim Present No Cost | Removes only from no cost best |
| | (continues) | | cost-benefit clinical data present those |
| | These | | recommended clinical data present |
| | parameters | | unable to increase P of diagnosis more |
| | affect only: | | than the parameter empirical value |
| | Data Cost | Trim Absent No Cost | Removes only from no cost best |
| | Quantity, | | cost-benefit clinical data absent those |
| | Abridged | | recommended clinical data absent unable |
| | Data Cost | | to decrease P of diagnosis more than the |
| | Quantity, | | parameter empirical value |
| | Parameter | Trim Present Greater Cost | Same as Trim Present No Cost but |
| | Affected | | applies only to small, intermediate, and |
| | Differential, | | great cost best cost-benefit clinical data |
| | Abridged | | present instead of no cost clinical data |
| | Parameter | Trim Absent Greater Cost | Same as Trim Absent No Cost but applies |
| | Affected | | only to small, intermediate, and great |
| | Differential, | | cost best cost-benefit clinical data absent |
| | Global | | instead of no cost clinical data |
| | Overview, and | Present Difference Cost | Compares P of best cost-benefit clinical |
| | Abridged | | data present in great cost category with |
| | Global | | P results achieved by best cost-benefit |
| | Overview | | clinical data in lower cost categories |
| | | | (no, small, and intermediate cost) and |
| | | | removes from the great cost best |
| | | | cost-benefit clinical data present those |
| | | | recommended clinical data unable to |
| | | | increase P of diagnosis an additional |
| | | | value, equal or greater than the value set |
| | | | for this parameter |
| | | Absent Difference Cost | Compares P of best cost-benefit clinical |
| | | | data absent in great cost category with |
| | | | P results achieved by best cost-benefit |
| | | | clinical data in lower cost categories |
| | | | (no, small, and intermediate cost) and |
| | | | removes from the great cost best |
| | | | cost-benefit clinical data absent those |
| | | | recommended clinical data unable to |
| | | | decrease P of diagnosis an additional |
| | | | value, set with this parameter |
| | | Cutoff Present | Establishes a P cutoff level that removes |
| | | | all recommended best cost-benefit |
| | | | clinical data assumed present (considered |
| | | | not worth to be investigated) for |
| | | | diagnoses with P below this level |
| | | Cuttoff Absent | Establishes a P cutoff level that removes |
| | | | all recommended best cost-benefit |
| | | | clinical data assumed absent (considered |
| | | | not worth to be investigated) for |
| | | | diagnoses with P above this level |
| | | Confirmation Threshold | Establishes an empirical P that confirms |
| | | | any diagnosis that reaches this value |
| | | Deletion Threshold | Establishes an empirical P that rules out |
| | | | any diagnosis that reaches this value |

| | | |
|---|---|---|
| OUTPUT | Comprehensive Differential Diagnosis List | Displays all differential diagnoses sorted |
| FILES | | by descending P calculated with |
| (continues) | | mini-max procedure, and all best |
| | | cost-benefit clinical data able to change |
| | | these P, sorted by cost category and |
| | | whether present or absent, showing |
| | | resultant P, were these clinical data to be |
| | | processed |
| | Differential Diagnosis List | Displays all differential diagnoses sorted |
| | | by descending P calculated with |
| | | mini-max procedure, the clinical datum |
| | | present that most favors each diagnosis, |
| | | and the clinical datum absent that most |
| | | disfavors diagnosis. It is a partial display |
| | | of Comprehensive Differential |
| | | Diagnosis List |
| | Global Overview | Best cost-benefit clinical data able to |
| | | change P of diagnoses after parameters |
| | | setting; data are grouped by cost, |
| | | displaying quantity in each category, |
| | | sub grouped by procedure to obtain them, |
| | | diagnosis to which each refer, presence |
| | | or absence, and resulting P for each |
| | | clinical datum |
| | Abridged Global Overview | Abridged version of Global Overview |
| | | with similar grouping, but displaying |
| | | only the best cost-benefit clinical datum |
| | | with greatest PP value and S in each |
| | | cost category, able to produce P change |
| | | after parameters setting |
| | Data Cost Quantity | Best cost-benefit clinical data able to |
| | | change P of diagnoses after parameters |
| | | setting; data are grouped by cost, |
| | | displaying quantity in each category, |
| | | but without further information |
| | Abridged Data Cost Quantity | Abridged version of Data Cost Quantity, |
| | | displaying only the best cost-benefit |
| | | clinical data with greatest PP value and S |
| | | in each cost category, able to produce |
| | | P change after parameters setting, |
| | | indicating quantity of data in each |
| | | cost category |
| | Data Cost Procedure Quantity | Best cost-benefit clinical data able to |
| | | change P of diagnoses after parameters |
| | | setting; data are grouped by cost, |
| | | displaying quantity in each category, |
| | | sub grouped by procedure to obtain them |
| | Abridged Data Cost Procedure Quantity | Abridge version of |
| | | Data Cost Procedure Quantity, |
| | | displaying only the best cost-benefit |
| | | clinical data with greatest PP value and S |
| | | in each cost category, able to produce P |
| | | change after parameters setting, |
| | | indicating quantity of data in each cost |
| | | category and procedure to obtain theni |
| | Parameter Affected Differential | Differential diagnosis list with all |
| | | diagnoses sorted by descending P, |
| | | but displaying best cost benefit |
| | | clinical data only for diagnoses with P |
| | | above deletion threshold, after parameter |
| | | setting |
| | Abridged Parameter Affected Differential | Abridged version |
| | | of Parameter Affected Differential, |
| | | displaying only the best cost-benefit |
| | | clinical data with greatest PP value and S |
| | | in each cost category, able to produce |
| | | P change after parameters setting |
| | Clinical Datum Lists | For each clinical datum with its number |
| | | and its cost, it lists all diagnoses that can |
| | | manifests it, showing S and PP value |
| | | of this datum for each listed diagnosis |
| | Numbered Data | Clinical data list sorted by their |
| | | identifying numbers assigned arbitrarily |
| | Complex Comprehensive | Displays all complex clinical |
| | | presentation models listing related |
| | | diagnoses, masking diagnoses, and |
| | | masking drugs with all best cost-benefit |
| | | clinical data able to change diagnoses P. |
| | | Total P of diagnoses are calculated with |
| | | mini-max procedure; diagnoses are |
| | | sorted by decreasing P and each disease |
| | | model is assigned a P equal to the |
| | | greatest P of the diagnoses in the model. |
| | | Cost-benefit clinical data are sorted by |
| | | cost category and whether present or |
| | | absent, showing resultant P, were these |
| | | clinical data to be processed. |
| | | The display for each individual diagnosis |
| | | in the complex clinical presentation |
| | | models is identical to the display for |
| | | the same diagnosis in Comprehensive |
| | | Diagnostic List, L, and R. M or |
| | | I: models; D: diagnosis; C: clinical datum |
| | Complex Short | Short version of |
| | | Comprehensive Complex, displaying only |
| | | related diagnoses, masking diagnoses |
| | | and drugs with respective P shown only |
| | | for diagnoses supported by clinical data |
| | | present; diagnoses not supported are |
| | | listed without further information |
| | SET OF | Differential diagnosis list with all |
| | Rxxxx-xxx-Dxxxx--<DIAGNOSIS> | diagnoses brought up by some clinical |
| | | data present. Each diagnosis is labeled |
| | | Rxxxx-xxx-Dxxxx--<DIAGNOSIS> |
| | | indicating from left to right: P rank, from |
| | | greatest (ordinal R0001) to the smallest |
| | | (greatest ordinal Rxxxx)-P value- |
| | | arbitrary order number--name. |
| | | Rxxxx-xxx-Dxxxx--<DIAGNOSIS> |
| | | provides for each diagnosis identical |
| | | information as |
| | | Comprehensive Differential Diagnosis |
| | | List does for the entire |
| | | differential diagnosis list |
| | SET OF | Complex clinical presentation models. |
| | Lxxxx-Rxxxx-Dxxxx--<DIAGNOSIS> | Each diagnosis is labeled |
| | | Lxxxx-Rxxxx-Dxxxx--<DIAGNOSIS> |
| | | indicating from left to right: P ranking of |
| | | model to which it belongs (equal to |
| | | the P of the greatest diagnosis in the |
| | | model)-P ranking of diagnosis-arbitrary |
| | | order number of diagnosis--name |
| | | of diagnosis. |
| | | Lxxxx-Rxxxx-Dxxxx--<DIAGNOSIS> |
| | | provides for each diagnosis identical |
| | | information as Comprehensive Complex |
| | | does for the entire set of complex clinical |
| | | presentation models. LXXXX (with |
| | | capitalized Xs) represents a diagnosis |
| | | that belongs to the model but is not in the |
| | | differential diagnosis list because so far |
| | | it has no supporting clinical data present |

### CAUTION

DO NOT CHANGE AND SAVE accidentally any input file; change and save them only if you want to correct or update intentionally some data, like when you enter clinical data present and absent in Present Data and Absent Data respectively, or your want to change parameters CHANGING AND SAVING any output file has no consequences because these files are regenerated at each iteration of the diagnostic program.

The two output files we like best are Global Overview and Abridged Global Overview, because they provide the most complete information to rationally select best cost-benefit clinical data and order the corresponding tests or procedures to efficiently reach end of diagnostic quest. These files display, in a single document, best cost-benefit clinical data hierarchally grouped by cost category, total quantity and partial quantity of clinical data in each cost category, test or procedure to obtain them, diagnoses affected by these clinical data, current P of each diagnosis in the differential diagnosis list, and expected P change that each displayed clinical datum will produce when processed for presence or absence. Sometimes, Data Cost Procedure Quantity and Abridged Data Cost Procedure Quantity may become handy for easier and faster transfer of these data to the input files Present Data and Absent Data files or to request the corresponding tests or procedures. Some other output files are predecessors.

Comprehensive Differential Diagnosis List file is not affected by parameter settings and is not a heuristically abridged output file. It may be useful in particular cases where the user is interested in a deeper insight in the diagnostic process and results, with all possible choices for clinical data selection, especially when patient's medical condition is critical. However, to get a synthetic overview of the differential diagnosis list, it is more practical to open shorter output files such as Index Files or Differential Diagnosis List, the latter displaying diagnoses sorted by decreasing order of P, each with its determining clinical data pair.

At first thought, abridged strategies bring up the concern that a diagnosis might be incorrectly ruled out because of lack of support by a smaller PP value best cost-benefit clinical data not recommended, in case the greater value datum were absent. However, this would happen only in the current diagnostic round; iterating the same strategy at further rounds will reach the convenient clinical datum. This strategy to recommend the exterior arrows by successive layers of gradually smaller PP value and S could be called “onion strategy”. Although it reduces the number of best cost-benefit clinical data to investigate, it could require some extra diagnostic rounds, with corresponding extra patient-physician encounters, if the diagnosis is not confirmed or ruled out at previous round.

When ascending in cost categories, the recommended best cost-benefit clinical data become scarcer, more exclusive for specific diagnosis, more supportive or eliminatory of diagnoses. The diagnostic process becomes clearer and the difference between the number of recommended best cost-benefit clinical data in abridged and corresponding non-abridged files diminishes considerably and even becomes equal. This parallels human diagnostic reasoning.

For abridged files we decided to leave the recommended best cost-benefit clinical data resulting in the greatest or smallest P in each cost category (exterior arrows of trichotomy tree) unaffected by any Trim parameter (Trim Present No Cost, Trim Absent No Cost, Trim Present Greater Cost, and Trim Absent Greater Cost). Furthermore, as this brought incongruence between non-abridged and abridged files, because clinical data listed in the latter were not listed in the former, considered more extensive, we added this exemption also to the non-abridged files, to preserve comparability with the abridged files. All remaining recommended best cost-benefit clinical data, increasing or decreasing P less than the mentioned clinical data, are affected by Trim parameters.

Best cost-benefit clinical data next to investigate are displayed in several output files—comprehensive Differential Diagnosis List, Global Overview, Abridged Global Overview, Data Cost Quantity, Abridged Data Cost Quantity, Data Cost Procedure Quantity, Abridged Data Cost Procedure Quantity, Parameter Affected Differential, and Abridged Parameter Affected Differential—some affected by parameters and others not. Best cost-benefit clinical data are sorted by increasing cost categories. In each such category, best cost-benefit clinical data assumed present and able to increase the current total P of the diagnosis are shown in decreasing order of resulting P, until no clinical data exists able to increase the current P. These clinical data are the ones at the top of the PP value list. Best cost-benefit clinical data assumed absent and able to decrease the current total P of the diagnosis are shown in increasing P value order, until no clinical data exists able to decrease the current P. These clinical data are the ones at the top of the S list.

“None” means that the program could not find any best cost-benefit clinical datum in the corresponding cost category, able to increase or decrease total P.

Each iteration of our program starts from the beginning, reprocessing all clinical data previously processed and newly ones entered in the computer; this is very convenient because it gives a chance to diagnoses, previously ruled out due to their small P, to reenter the competition, if the new clinical data confer them a greater P. Recalculated P for all diagnoses selected by all clinical data present, and all best cost-benefit clinical data able to change these P, are displayed in Comprehensive Differential Diagnosis List.

Stratagem for Databases that Do Not Include All Known Diseases and Clinical Data

Our program confirmed the importance of the exhaustiveness condition for calculating P of diagnoses, which states that to obtain accurate results all known disease models must be included in the database. Because we were not able to integrate such an extensive database on our own, we resorted to a stratagem, creating a fictitious disease model that we called OTHER DISEASES in addition to the real 50 disease models that integrate our prototype model. This OTHER DISEASES model represents all other known diseases (estimated at several thousands). Without this artifact, the computer program interpreted some irrelevant clinical datum, for example faint heart sounds, as exclusive for pericarditis with effusion, simply because this clinical datum was not present in the remaining 49 diagnoses. Without OTHER DISEASES, equation 5 that calculates PP value of the mentioned clinical datum, had S=0.50 in the numerator and S=0.50 in the denominator being S=0 for all other diagnosis, yielding a PP value=1.00 resulting in an improperly confirmatory P=1.00 for the diagnosis of pericarditis. By creating OTHER DISEASES model and including in its long clinical data list the clinical datum “faint heart sounds” with a great S, we precluded this situation to occur. This great S added to the denominator of equation 5, reduces considerably the PP value of the mentioned clinical datum for pericarditis and P of this diagnosis to a non-confirmatory level.

However, at this point, another problem surfaced. In OTHER DISEASES, when assigning a great S to a clinical datum (e.g., S=1.00) that happens to be absent for other diagnoses, this S will integrate the corresponding terms in the denominator of equation 8 that calculates P of diagnoses. The corresponding term [PP value (1−S)]=[PP value (1-1)]=0. One or more terms equaling 0 in the denominator will incorrectly increase considerably P of the diagnosis being processed. To neutralize this untoward effect, we had to create an extra OTHER DISEASES SAME model in addition to the OTHER DISEASES model, repeating in both models the same clinical data but assigning to each corresponding S half of its original value. Because these S values are added in the denominator of equation 5 that calculates PP values, the resulting PP value of a specific clinical datum for a specific diagnosis with half S value in both models, will be the same as with only OTHER DISEASES with S equal the original entire value. However, an excessively great P is precluded by processing both mentioned models, because now the term [PP value (1−S)]=[PP value (1-0.50)] will yield a greater value and will appear twice in denominator of equation 8. Our program hides OTHER DISEASES and OTHER DISEASES SAME diagnoses form showing in the differential diagnosis list and other output files.

Applying principles similar to above paragraph, we can preclude diseases that are gender exclusive to be misdiagnosed for the opposite gender. For instance in the disease model ectopic pregnancy, we include the clinical datum female with S=1.00 and the same clinical datum with S=0.50 in both OTHER DISEASE and OTHER DISEASES SAME models. Initially, a certain P will be calculated for ectopic pregnancy, but at very next algorithm iteration, the no cost best cost-benefit clinical data female will be recommend in first place and as soon as entered in Absent Data (because the patient is male), the P of ectopic pregnancy will drop to 0 and the diagnosis ruled out. Similarly, for male diseases such as prostatic carcinoma, we include in its disease model the clinical datum male with S=1.00 and the same clinical datum with S=0.50 in both OTHER DISEASES and OTHER DISEASES SAME models.

### Complex Clinical Presentations and Their Models

The diagnostic process comprises several levels of complexity. Related clinical data cluster to a syndrome, simple syndromes comprising only a few clinical data coalesce to a complex syndrome or disease, and sometimes to a yet more complex clinical presentation (page 10), where the relation of clinical data becomes less obvious.

The algorithm thus far presented uses probabilistic calculations, with mini-max procedure, best cost-benefit clinical datum, and discrimination between competing diagnoses and concurrent diagnoses, to determine the P of a final diagnosis. It will work well with simple clinical entities, such as uncomplicated diseases or syndromes where clinical data typically are interrelated and linked to a single cause or lesion. Examples of such simple diseases or clinical entities include bronchitis, asthma, gastroenteritis, hyperthyroidism, obstructive jaundice, and renal failure. At this diagnostic stage, a single final diagnosis accounts for all manifested clinical data.

In an actual patient, the clinical picture might be more complicated; as a fact, severely ill intensive care unit patients often have multi-organ involvement, present multiple and proteiform clinical data, and may mandate consultation by several specialists. For example, coronary artery disease, acute myocardial infarction, congestive heart failure, shock, and thromboembolism in a single patient. A specific disease can manifest diverse clinical forms and clinical presentations, complicating the diagnostic process. This situation makes impossible to determine the S of each clinical datum for the entire complex clinical presentation because this would involve multiple clinical forms, concurrent diseases, and multiple pathogenic and pathophysiologic mechanisms. It would require analyzing a statistically significant number of cases with identical combinations of clinical entities; it also would take us into an exponential or NP-complete computational time and complexity. Accordingly, probabilistic methods are unsuitable for processing any complex clinical presentation; indeed, to my knowledge, no commercial diagnosis programs that can accomplish this exist. A categorical method for processing complex clinical presentations is simple and feasible.

For this reason our algorithm, with its heuristic principles and moderate use of probability, diagnoses first only relatively simple syndromes, clinical entities, or diseases. Let the diagnostic algorithm produce as many final diagnoses of simple concurrent diseases, syndromes, complications, etc. as the clinical data dictate. Our algorithm is able to diagnose satisfactorily these simple clinical entities and also to recognize concurrent diseases or clinical entities. Once we have these partial components (clinical entities), the database must offer categorical models (complex clinical presentation models; see below), one for each possible clinical interrelation or association of these entities: causal relations, evolutive stages, complications, severity, type, localization, etc. Such clinical presentation models, although numerous, are not excessive, and are described in any authoritative medical textbook. This diagnostic stage does not require a probabilistic approach, but a pure categorical one.

Categorically relating clinical entities based on their associated pathophysiologic links or statistical correlations, into a complex clinical presentation mandate creating a specific model for each possible combination. We call these models complex clinical presentation models.

A complex clinical presentation model comprises related clinical entities and diseases; clinical data are excluded from this definition because they are elements of a disease model.

Many prior art algorithms employ tree and network structures that extend from cause of disease to clinical data and vice versa, placing probabilities on nodes, branches, and leaves. Most such structures are complex and required years to assemble. We suspect that such structures are difficult to update and would need to be redesigned every few years. In contrast, our algorithm can relatively easily be updated at any time, by simply updating in disease models the S values of clinical data, adding or deleting clinical data when necessary, or adding or deleting disease models.

In summary, the entire diagnostic process is achieved in 2 steps:

Step 1. Probabilistic processing of clinical data matches patient clinical data with clinical data in disease models yielding a differential diagnosis list. Mini-Max Procedure, best cost-benefit clinical data next to investigate, and discrimination between competing diagnoses and concurrent diagnoses achieves as many concurrent final diagnoses of clinical entities as are required to account for all manifested clinical data. Then, the algorithm proceeds to Step 2.

Step 2. Categorical processing of clinical entities matches confirmed final diagnosis with all complex clinical presentation models in the database. If a match is found, all the related diagnoses in this model are included in the differential diagnosis list to be processed in the usual way by min-max procedure and recommended best cost-benefit clinical data, being confirmed or ruled out. The same complex clinical presentation models enable establishing whether concurrent diagnoses are related or unrelated, when respectively a linking model exist or not.

### Complex Clinical Presentations Managed By our New Program

Complex clinical presentation models are categorical combinations of clinical entities linked by pathophysiologic or statistically significant relations; they can be created, displayed, and modified with input file Complex Presentation Models, which are part of the database. These models have at least three functions: (1) preclude overlooking diagnoses; (2) manage interactions (masking) among diseases and drugs, and (3) distinguish related from unrelated concurrent diagnoses.

### 1. Associated Diagnoses

The purpose of this function is to preclude overlooking diagnoses, which might be of crucial importance for the global treatment of a patient, when no clinical data present supporting them have been entered so far in the computer. Such diagnoses are suggested by association with confirmed diagnoses in the complex clinical presentation model, and will be processed even if not included yet in the differential diagnosis list. This is achieved through the following steps:

1. Create complex clinical presentation models—Complex Presentation Models—listing in each, all diagnoses that present a possible pathophysiologic or statistically significant link (diseases, causes, complications, related diagnoses, etc.). Our program, assigns to each complex clinical presentation model a letter M, a number (Mxxxx), and an appropriate title (e.g., CARDIOVASCULAR). Each diagnosis in the model has its letter D, corresponding number (Dxxxx) and name (e.g., AORTIC DISSECTION, MYOCARDIAL INFARCTION, PULMONARY EMBOLISM . . . ).
2. Create in the database—Disease Models—disease models for the diagnoses mentioned in the previous paragraph, with their corresponding clinical data and sensitivities (S), if these models are not already included, and run Datum Program.
3. Enter patient's clinical data present and absent in respective Present Data and Absent Data files and run the Diagnostic Program.
4. After the necessary program iterations, for each confirmed final diagnosis, the algorithm searches all complex clinical presentation models for a match of this confirmed diagnosis with at least one similar diagnosis in the mentioned models. If such a match is established, all the linked diagnoses of the model are included in the differential diagnosis list, if not already included, to be processed for presence or absence. Best cost-benefit clinical data for these diagnoses will be recommended and once selected and investigated, enter them in respective Present Data and Absent Data files and run Diagnostic Program again.
5. The diagnostic program—Diagnostic Program—will calculate P of each diagnosis listed in the complex clinical presentation model. Diagnoses inside the model are sorted by decreasing P, the greatest of these P is assigned as P of the entire model, and models are also sorted by decreasing P. Each linked diagnosis is processed with the usual mini-max procedure, to become a confirmed concurrent diagnosis or to be ruled out. The result of the process is displayed in the output files Complex Short that shows linked diagnoses with their P, and Complex Comprehensive that shows linked diagnoses with their P and best cost-benefit clinical data recommended for further processing. Linked diagnoses that at the current diagnostic iteration have no supporting clinical data present, will not show P, because this value must be calculated based on such clinical data, but will show the recommended best cost-benefit clinical data in Complex Comprehensive. Index Files lists all these diagnoses, labeled as Lxxxx-Rxxx-Dxxxx-<DIAGNOSIS>. The format of these L files was discussed earlier.

The diagnostic quest requires processing only diagnoses related to confirmed diagnoses (final diagnoses); calculations of P for diagnoses related to non-confirmed diagnoses would be too numerous, cumbersome, and irrelevant. Nevertheless, our program offers both options: (1) Complex Comprehensive output file recommends best cost-benefit clinical data for diagnoses in complex clinical presentation models related to all the diagnoses in the differential diagnosis list, confirmed or not, leaving to the user decide to which level he wants to process such diagnoses. (2) Global Overview, Abridged Global Overview, and other parameter sensitive output files recommend best cost-benefit clinical data for diagnoses in complex clinical presentation models related only to diagnoses that reached confirmatory threshold.

Those diagnoses in a matched complex clinical presentation model that were not included yet in the differential diagnosis list by previously collected clinical data, must be included by their links to confirmed diagnoses. Their probabilities are calculated in the usual way with mini-max procedure, but not being supported by any previously collected clinical datum, this calculation must rely exclusively on best cost-benefit clinical data. Consequently, information of greatest PP value and S of nonexistent previous supporting clinical data, otherwise displayed between the diagnosis title and the best cost-benefit clinical data in Complex Comprehensive and L files (labeled RXXX), is missing for these diagnoses. This information will be displayed only after at least one of the recommended best cost-benefit clinical data is entered in Present Data input file, and the Diagnostic Program is run again. When some of these diagnoses reach a confirmatory P, they become concurrent diagnoses.

We confirmed that diagnoses, whose P is calculated probabilistically with mini-max procedure, must be kept simple and pure (not contaminated with causes, complications, etc.); when two or more diagnoses are confirmed, they must be combined categorically. An example of how violation of this rule affects results follows: acute aortic dissection sometimes produces the complication myocardial infarction; because of this fact, we erroneously included increased troponins as a clinical datum for acute aortic dissection, when it actually is an exclusive clinical datum for myocardial infarction. As a result, increased troponins incorrectly confirmed acute aortic dissection. Troponins should have been listed only as a clinical datum for myocardial infarction; acute aortic dissection and myocardial infarction should have been diagnosed as concurrent clinical entities, and then be linked categorically as cause and complication, by matching the corresponding complex clinical presentation model they share.

A match of a confirmed diagnosis (e.g., emphysema) with only one similar diagnosis in a complex clinical presentation model suffices to select this model and include all its related diagnoses in the differential diagnosis list, even if only one of them (e.g., pneumothorax) may be confirmed as a concurrent complication.

### 2. Disease and Drug Interactions (Masking)

Drugs often interact, one enhancing or reducing the effects of another. Drugs also may adversely alter clinical data of a disease. In a somewhat similar manner, concurrent diseases may interact, one reducing (masking) or less frequently enhancing a clinical datum of another. Let's consider some examples:
Chest pain of acute myocardial infarction may be masked by concurrent diabetes, strong analgesics, or advanced age.
A positive tuberculin reaction may be rendered negative by a concurrent immunosuppressive disease (AIDS) or a drug (e.g., a corticosteroid).
A systolic hypertension may be reduced by concurrent acute myocardial infarction or shock.
Inflammatory symptoms of rheumatic diseases or appendicitis may be suppressed by corticosteroids or antibiotics.
Diseases that affect liver function are able to produce a false negative cholecystogram, even with a normal gallbladder, because of the incapacity of the liver to concentrate the contrast media. This case should be considered a masking situation, where a liver disease masks or cancels a clinical datum for a normal gallbladder, and should be processed accordingly.
Typical hypophosphatemia of primary hyperparathyroidism is masked by a concurrent renal failure, produced by this disease, raising phosphate to a false normal level.

Disease and drug interactions are dangerous, because they can mask important clinical data and result in misdiagnosis. This is especially important for diagnosis of life threatening diseases.

The affected clinical datum in general is diminished in intensity or completely masked, as in the above examples; we are dealing with a clinical datum absent that would otherwise be present in the disease. In our diagnostic algorithm, the absence of an expected clinical datum tends to rule out the disease in direct proportion to the S of the datum. In the first example, chest pain in acute myocardial infarction has a great S (occurs frequently). With the Mini-Max Procedure, absence of chest pain, a consequence of concurrent diabetic neuropathy, would greatly reduce the P of myocardial infarction and could have dismal consequences. Accordingly, if a concurrent disease cancels a clinical datum of the primary disease, the S of this clinical datum must be proportionally reduced, to diminish its rule-out power. A practical solution is to consider chest pain S=0 whenever myocardial infarction is suspected in a diabetic patient; this is equivalent to elimination of chest pain from diagnostic consideration. In this case the diagnosis of myocardial infarction must be achieved with other clinical data present such as an ECG and cardiac enzymes.

Masking occurs infrequently; therefore only those diagnoses and clinical data known to be susceptible to masking are processed for interaction. Clinical data present, either initially collected or recommended as best cost-benefit clinical data, obviously are not masked. Masking refers to a clinical datum absent, posing a dilemma whether it is genuinely absent or masked by a concurrent disease or drug. Each clinical datum susceptible to be masked has associated a list of drugs and diseases able to mask it; if recommended as best cost-benefit clinical datum assumed absent, it comes from the S list. Only clinical data absent with great S are relevant because only they significantly reduce P of a diagnosis. Summarizing, a clinical datum potentially masked must be detected, have a great S for the corresponding diagnosis, and found absent. When a clinical datum absent of great S is processed, the algorithm checks whether it is susceptible to be masked. If so, potentially interacting diagnoses are added to the differential diagnosis list to be confirmed or ruled out, and the user is asked whether the patient is receiving specific drugs capable of interaction. If any of these drugs or diagnoses is confirmed, S of the clinical datum susceptible to be masked is reduced to zero, which is equivalent to delete it from the corresponding diagnosis, and total P of the diagnosis is calculated with other clinical data present, but a new column for the datum absent should be generated in all mini-max tables because the same clinical datum may not be masked in other diagnoses. When a clinical datum assumed absent is found present, it is disregarded and no new column is generated in mini-max tables.

Because only certain specific clinical data of specific diseases are susceptible to be masked by specific concurrent diseases or drugs, prior algorithm of our main patent flags such clinical data in the corresponding disease models and lists the potential masking diseases and drugs.

Our new algorithm handles the problem in a way that is similar to the complex clinical presentations described above, because some related concurrent diseases or drugs with masking property must be processed for presence or absence. With our new diagnostic program, they are included in specific complex clinical data models together with the diagnosis that comprises the clinical datum susceptible to be masked, managing masking through the following steps:

1. Create a complex clinical presentation model—in Complex Presentation Models input file—for each diagnosis comprising a clinical datum susceptible to be masked (e.g., chest pain of myocardial infarction, which can be masked by concurrent diabetes, potent analgesics, or advanced age). The model, numbered Ixxx, is given an appropriate title (e.g., MYOCARDIAL INFARCTION WITH MASKED CHEST PAIN) and includes the following items processed like potentially concurrent diagnoses, numbered Dxxxx.: (1) Diagnosis with a potentially masked clinical datum (e.g., MYOCARDIAL INFARCTION WITH MASKED CHEST PAIN), (2) The potentially concurrent diagnoses (DIABETES, MASKING DRUGS, ADVANCED AGE) that, if confirmed, can mask the clinical datum (chest pain). Creating these complex clinical presentation models including the diagnoses with clinical data susceptible to be masked, is equivalent to flagging these diagnoses and clinical data.
2. Include, if not already included, in the database—Disease Models—disease models for the diagnoses mentioned in the previous paragraph, with their corresponding clinical data: MYOCARDIAL INFARCTION WITH MASKED CHEST PAIN, DIABETES, ADVANCED AGE, and MASKING DRUGS, the latter considered a diagnosis; a single drug is sufficient to confirm this clinical datum and diagnosis. Now, the database includes the original myocardial infarction without masked chest pain and the added myocardial infarction with masked chest pain, the latter disease model omitting this clinical datum (chest pain).
3. Run the diagnostic program—Diagnostic Program. When supporting clinical data present bring up a diagnosis with a clinical datum susceptible to be masked, the differential diagnosis list—Comprehensive Differential Diagnosis List—will display two similar competing diagnoses: <DIAGNOSIS WITHOUT MASKING> and <DIAGNOSIS WITH MASKING>. However, a problem results at this point: the confirmatory clinical datum (e.g., increased troponins with S=1.00) of acute myocardial infarction without masking competes with the same confirmatory clinical datum of myocardial infarction with masked chest pain, and this yields a total P of only 0.50 for each, instead of 1.00 for one of them. To preclude this from occurring, we resort to an artifact, adding in the original disease model in the database (MYOCARDIAL INFARCTION WITHOUT MASKED CHEST PAIN) the confirmatory clinical datum without masking (increased troponins without masked chest pain) to the already existing confirmatory clinical datum (increased troponins). Similarly, we add in the disease model with masking (MYOCARDIAL INFARCTION WITH MASKED CHEST PAIN) the confirmatory clinical datum with masking (increased troponins with masked chest pain) to the already existing confirmatory clinical datum (increased troponins). All these clinical data have the same S. For clinical data with small S it is not necessary to repeat these clinical data with and without masking, because they have no relevance in confirming or ruling out diagnoses. Conversely, every clinical datum with great S, having greater ruling out power (P reaching deletion threshold), must be duplicated the way mentioned above.

Once both competing diagnoses-DIAGNOSIS WITHOUT MASKING> and <DIAGNOSIS WITH MASKING—are in the differential diagnosis list, Global Overview, Abridged Global Overview, Data Cost Procedure Quantity, and Abridged Data Cost Procedure Quantity will recommend both best cost-benefit clinical data: increased confirming clinical datum with masked clinical datum (increased troponins with masked chest pain) and increased confirming clinical datum without masked clinical datum (increased troponins without masked chest pain). Which of these two recommended data must be selected and entered in Present Data depends on whether at least one of the masking diagnoses was confirmed final or not.

4. Now, it is necessary to establish whether a masking diagnosis can be confirmed. If a diagnosis able to mask a clinical datum of another diagnosis—as establish by Complex Presentation Models—is already included in the differential diagnosis list, supported by at least one clinical datum present, it will automatically be processed. If not included in the differential diagnosis list, to process the potentially masking diagnosis requires opening Complex Comprehensive or Complex Short and investigating the recommended best cost-benefit clinical data. However, if no supporting clinical data for this diagnosis were collected so far, all its clinical data (listed in the corresponding disease model) will be listed as recommended best cost-benefit clinical data sorted by cost category and are expected to be quite numerous. These clinical data are entered in Present Data and Absent Data respectively and saved; the program—Diagnostic Program—is run again. Masking diagnosis and its P will be displayed now in the differential diagnosis list.
5. If any masking diagnosis is confirmed (DIABETES, MASKING DRUGS, or ADVANCED AGE), we enter the confirmatory datum with masking (increased troponins with masked chest pain) in the list of clinical data present—Present Data. If no masking diagnosis is confirmed, we enter the confirmatory datum without masking (increased troponins without masked chest pain) in the list of clinical data present—Present Data.
6. The diagnostic program—Diagnostic Program—is run again and the result—in Comprehensive Differential Diagnosis List, Complex Comprehensive, and several other output files—will confirm one (MYOCARDIAL INFARCTION WITHOUT MASKED CHEST PAIN) or the other (MYOCARDIAL INFARCTION WITH MASKED CHEST PAIN) of the two competing diagnoses.

Without flagging, how do we know which clinical data are susceptible to be masked and which diagnoses include them? There are three clues:
1. Two similar diagnoses competing in the differential diagnosis list.
2. Their denomination (with and without masking).
3. Complex clinical presentation models Complex Presentation Models, Complex Comprehensive, and Complex Short list the mask and masking diagnoses.

For associated diagnoses we process all related diagnoses only to confirmed diagnoses (those diagnoses with P equal to or greater than the Confirmation Threshold); the best cost-benefit clinical data of the associated diagnoses are recommended in the corresponding output files, together with the ones for all other differential diagnoses. Instead, for disease and drug interactions (masking) we process all masking diagnoses only for those potentially masked diagnoses with P equal to or greater than the Cutoff Present parameter (with reasonable chance to become confirmed by other supporting clinical data). The best cost-benefit clinical data of the masking diagnoses are recommended in the corresponding output files, together with the ones for all other differential diagnoses. The confirmation of a masking diagnosis and the deletion of the masked clinical datum will increase P of the masked diagnosis, precluding the latter from being ruled out, were its P unduly reduced by the masking diagnosis.

### 3. Related and Unrelated Concurrent Diagnoses

Complex clinical presentation models have still another function: enabling to distinguish related from unrelated concurrent diagnoses. When two or more concurrent diagnoses are included in a single complex clinical presentation model, by definition these diagnoses are related. Conversely, if no single model exists that includes (relates) the concurrent diagnoses, they are unrelated.

### Diagnosis by Exclusion

When clinical data of great PP value that strongly support a diagnosis are unavailable or too costly, the diagnostic process comes to a standstill. The user then can resort to diagnosis by exclusion where, from a group of competing diagnoses, all but one are excluded. The single remaining diagnosis is accepted as a final diagnosis, even though its P is relatively small. All other diagnoses would have been eliminated because the great S of corresponding clinical data absent would have reduced their P below the elimination threshold. For example the diagnosis of acute appendicitis is acceptable when all other causes of acute right lower quadrant abdominal pain and tenderness have been excluded.

### Empiric Treatment

If two or more diagnoses remain in the differential diagnosis list, all of which are likely to respond to a single treatment, an empiric treatment may be warranted. If such treatment is successful, a more accurate but costlier final diagnosis will be unnecessary. For example, when only a few rheumatic diagnoses remain in the differential diagnosis list, empiric treatment with corticosteroids may be justifiable.

### Comparison of Two Algorithms

Comparing prior algorithm of main patent application with current alternative algorithm and program of this continuation-in-part, we noticed some tradeoff existing between them.

### Current Program:

Current program does not strictly follow prior algorithm; it has the following characteristics:

1. It reprocesses from start, after each new clinical data input, the entire gamut of functions of our diagnostic process. This includes P recalculation of single diagnoses or concurrent diagnoses, recommendation of sets of best cost-benefit clinical data, complex clinical presentations, associated diagnoses, and masking. This proved to work efficiently. Before that, we had some doubts from which reentry point each specific iteration had to start.
2. It has the advantage to calculate and display at once all the best cost-benefit clinical data able to change P of diagnoses, were these clinical data found present or absent. Before that, we had some doubts about how many of these clinical data from the top of the PP value list and S list should be displayed. The comprehensive display has the advantage to provide a total overview of best cost-benefit clinical data to consider; restrictive strategies—Parameters and Abridged output files—as described earlier, facilitate selection of a set of them. This program showed that the number of clinical data displayed is not unmanageable (at least for the present limited database).
3. It processes masking in a somewhat cumbersome way, but works efficiently and takes advantage of the general structure of the algorithm, without the need to write specific routines for this function.
4. It works nicely and fast with our current database including only 50 diseases. Some uncertainty remains regarding duration of computational time once the database will be integrated with thousands of diseases and clinical data. However, intuition tells us that this computational time will not increase proportionally to the number of diseases and clinical data, because the degree of shared clinical data, which burdens the diagnostic process, does not increase proportionally to the number of diagnoses.

### Prior Algorithm:

Proposes more specific routines for each function, being more sequential, intuitive, and didactic, without iterating from the very start for each change in the input data; it might be more efficient and computer time saving once the database will be integrated with thousands of diseases and clinical data.

### Steps of our Diagnostic Program

1. Before processing a new clinical case or creating one, all previous clinical data in file Present Data and in file Absent Data must be deleted and the empty files saved. If the previous case with its clinical data is desired to be preserved unmodified, it has to be Saved As with an appropriate name; the new case must be given a different name to avoid overwriting previous files.
2. Initially collected clinical data, present or absent, must be searched with Find function in any of the files-Data Procedures, Disease Models, Clinical Datum Lists, or Numbered Data—that list all the clinical data with their respective arbitrary number (Cxxxx), copied and pasted, side by side, into the respective input files. Because our program processes clinical data based only on the corresponding Ccxxxx number, when copying and pasting clinical data, always include this number. This also enables processing synonyms, which rely on the clinical data numbers. Our recommendation, at this step, is to enter only initially collected clinical data present in Present Data, and none of the absent ones. Clinical data present initially collected through medical history, are quite reliable because the patient is aware of them, and so are clinical data obtained through physical examination by a responsible physician. Clinical data absent obtained from medical history are (1) subjective and unreliable. Frequently, patients asked for specific symptoms tend to answer affirmatively, when actually absent; some apprehensive patients even tend to answer affirmatively to all symptoms asked. The opposite also happens as in the following example: a patient with a pancreatic carcinoma presented with a frank obstructive jaundice at physical examination, confirmed with further tests; however, at initial interrogation, he denied dark urine and light colored stools. These statements were erroneous simply because he had not paid attention to these obvious signs. Processing these clinical data as absent creates a contradiction that may result in an excessive reduction of diagnosis P. (2) Clinical data absent do not select new diagnoses. (3) Too many clinical data absent are documented with the clinician's traditional and detailed review of symptoms by body regions, organs, or systems. (4) Many data absent do not correspond to diagnosis included in the differential diagnosis list; processing them is a futile cost and effort. (5) in further diagnostic rounds more appropriate clinical data absent to investigate will be recommended by the best cost-benefit function, (6) clinical data absent are more cumbersome at initial collection, to be searched with Find function, copied and pasted into Absent Data, whereas at further rounds this process will be much easier because recommended clinical data will already be listed in a single file and need only to be copied and pasted in Absent Data. After this initial diagnostic round, recommended best cost-benefit clinical data absent must be investigated and entered in the computer to further reduce P of competing diagnoses or rule them out.
3. Run program (each time a change is made in the input files). Two kinds of clinical data are processed differently by our program: (1) Clinical data investigated and confirmed present or absent in the patient, entered respectively in Present Data and Absent Data; these data are processed calculating partial P for all the cells of the mini-max tables. (2) Best cost-benefit clinical data, recommended but not yet investigated; these data are processed with the shorter and simpler method to calculate only one significant cell for each diagnosis—the determining partial P—equal to total expected P of the diagnosis, were this datum investigated. The shorter method to calculate P disregards broken monotony, not possible to visualize in the mini-max table showing only one cell (partial P), although it is detected in the recommended best cost-benefit clinical data present and absent lists by the disrupted progressive reduction of respective PP values and S (page 58). The heuristic simplification of the shorter method has no major impact on the accuracy of the resultant P because it applies only to recommended clinical data next to investigate; once investigated and entered in the computer, the following program iteration, which calculates all partial P, will yield a more accurate diagnosis P. Furthermore, the difference of P calculated with all cells (involving broken monotony) or only one (not involving broken monotony) is of small magnitude.
4. Re-interrogate and reexamine the patient for no cost best cost-benefit clinical data present and absent recommended by Abridged Global Overview, and enter results in Present Data and Absent Data respectively. Here, the technique of copying and pasting, to transfer these data from recommending output list to input files also applies. Abridged Global Overview output file is shorter than Global Overview; the result of ordering only the clinical data in the shorter file apparently yields the same accuracy as ordering the ones in the longer file.
5. Run program. All the steps mentioned up to here can be done at the initial patient-physician encounter. After this second iteration, if diagnostic quest is not concluded yet, the remaining recommended no cost best cost-benefit clinical data typically can do too little for changing significantly P. It is now time to resort to greater cost category clinical data and order recommended small cost and/or even intermediate cost clinical data if necessary to reach a significant P of diagnoses. Select and order tests or procedure recommended in mentioned cost categories by Abridged Global Overview and schedule next patient-physician encounter.
6. At next patient-physician encounter enter results of tests and/or procedure in corresponding Present Data or Absent Data.
7. Run program. If diagnostic quest is not concluded yet, select and order tests or procedures from great cost clinical data recommended by Abridged Global Overview or Global Overview and schedule next patient-physician encounter.
8. At next patient-physician encounter enter results of tests and/or procedure in corresponding Present Data or Absent Data.
9. Run program. If diagnostic quest is not concluded yet (unusual), step 9 can be repeated with next great cost clinical data now recommended by Global Overview. This process is iterated until final diagnoses are achieved, the cost of confirming new best cost-benefit clinical data becomes prohibitive, or all clinical data are exhausted. If final diagnoses are not reached, resort to diagnosis by exclusion. When a diagnosis does not reach confirmation threshold, but qualifies for diagnosis by exclusion (all other diagnoses in the differential diagnosis list have been ruled out), this confirmation threshold can be lowered to the P value of this diagnosis in order to convert it into a confirmed diagnosis. This will activate complex diseases models function and display of best cost-benefit clinical data corresponding to associated diagnoses.

At this point Complex Presentation Models have been automatically processed and corresponding best cost-benefit clinical data have been recommended. If associated diagnoses or masking diagnoses were applicable, they were already processed; if masking drugs were questioned, the user was already presented with a list of such drugs.

Typically, not all recommended greater cost clinical data are selected and ordered at once; there is a tradeoff between cost of reaching a sooner conclusion of the diagnostic quest and the number of necessary patient-physician encounters to complete the diagnostic quest. The decision of how many and type of tests to order at each encounter depends on socio-economic and personal situation of each patient.

While our above description contains many specificities, these should not be construed as limiting the scope of the invention but as merely providing illustrations of some of the presently preferred embodiments of this invention.

Accordingly, the scope of the invention should be determined by the appended claims and their legal equivalents, rather than by the examples given.

What is New in this Continuation-in-Part?

In addition to the several novel inventions of the main patent application, this continuation-in-part application adds but is not limited to:
A new alternative diagnostic algorithm, tested with our computer program, described with corresponding flow chart; this algorithm is based on the same general principles of the prior main application and performs the following novel functions in addition to the previous ones:
Complete display of recommended best cost-benefit clinical data capable to change P of diagnoses; after each program iteration ample information for analysis of diagnostic process is offered to the user.
Entering in Present Data or Absent Data any best cost-benefit clinical datum supersedes and removes from the same cost category all other recommended data that produce a smaller change of P, reducing considerably the number of data to investigate.
Grouping clinical data according to test or procedure necessary to investigate them, facilitating their request.
Parameters that provide diverse strategies to reduce number of best cost-benefit clinical data to investigate, without compromising accuracy of diagnostic process.
Abridged output files that reduce number of recommended best cost-benefit clinical data to investigate, without compromising accuracy of diagnostic process.
OTHER DISEASES and OTHER DISEASES SAME that enable to create an efficient program with a limited number of diseases and clinical data in the database, overcoming the condition of exhaustiveness stating that all known diseases and clinical data must be included in the database.
Diverse output files that facilitate selection of a set of best cost-benefit clinical data to investigate simultaneously, by recommending these data organized by cost category, procedure to obtain them, quantity, diagnosis, and P of diagnoses before and after processing these data whether present or absent.
Complex clinical presentation models for associated diagnoses and interaction (masking) diagnoses, precluding overlooking of diagnoses
Iteration of the entire program from start, each time a new clinical datum present or absent is entered in the computer, providing a chance to diagnoses ruled out at previous iterations to reenter the competition when new supporting clinical data present increase P of such diagnoses.
A novel and simple method to handle synonyms of diseases and clinical data.
Distinguishing related and unrelated concurrent diagnoses, based on complex clinical presentation models.

1. A medical diagnostic algorithm excluding disease prevalence and subjective qualities of clinical data, said algorithm comprising the steps of:
a) computing for each clinical datum a sensitivity S based on disease cases to derive sensitivities as follows:
$$S = \frac{\begin{matrix}number of disease cases \\ manifesting said clinical datum\end{matrix}}{total number of diseases cases} ;$$
b) computing for each clinical datum present a positive predictive value PP valueᵢ supporting a disease diagnosis i, thereby obtaining PP value₁ through PP valueₙ:
$$PP {value}_{i} = \frac{{S}_{i}}{{S}_{1} + … + {S}_{i} + … + {S}_{n}} ,$$
where Sᵢ denotes sensitivity of said clinical datum present for said disease diagnosis i and S₁ . . . Sₙ denote sensitivities of said clinical datum for corresponding disease diagnoses 1 . . . n;
c) storing said sensitivities and said positive predictive values in a database linked to each said clinical datum and to a corresponding disease model;
d) ruling in disease diagnoses into a differential diagnosis list when clinical data present in a patient match at least one clinical datum present for said corresponding disease model;
e) establishing a cost of collecting a clinical datum not yet investigated in said patient, said cost of collecting being the maximum value of expense, risk and discomfort to said patient:
Cost=max(expense,risk,discomfort);
f) establishing a benefit of collecting said clinical datum not yet investigated in said patient based on its impact on a probability P of at least one of said disease diagnoses in said differential diagnosis list;
wherein said clinical datum not yet investigated is chosen for investigation in said patient in an analysis comprising a best cost-benefit consideration of said cost of collecting and said benefit of collecting.

2. The algorithm of claim 1, further comprising grouping said clinical datum not yet investigated with additional clinical data not yet investigated based on said cost of collecting into cost categories.

3. The algorithm of claim 1, further comprising grouping said clinical datum not yet investigated with additional clinical data not yet investigated based on clinical procedures associated with collecting said clinical datum not yet investigated and said additional clinical data not yet investigated.

4. The algorithm of claim 1, wherein said probability P for each of said disease diagnoses for said patient equals a maximum value among PP value₁ through PP valueₙ for each of said disease diagnoses:
P=max(PP value₁,PP value₂, . . . , PP valueₙ).

5. The algorithm of claim 4, further comprising:
a) determining probabilities Pᵢ for select clinical data, said select clinical data comprising said clinical datum not yet investigated and additional clinical data not yet investigated;
b) retaining among said select clinical data only those clinical data producing a predetermined change in probability P of corresponding diagnosis, thereby reducing the total number of clinical data not yet investigated to be included in said best cost-benefit consideration.

6. The algorithm of claim 1, further comprising parametrizing said clinical datum not yet investigated in view of additional clinical data not yet investigated.

7. The algorithm of claim 6, wherein said parametrizing comprises selecting at least one parameter for said clinical datum not yet investigated and said additional clinical data not yet investigated, and assigning a value to said at least one parameter.

8. The algorithm of claim 7, wherein said at least one parameter is selected from the group of parameters consisting of trim present no cost, trim absent no cost, trim present greater cost, trim absent greater cost, present difference cost, absent difference cost, confirmation threshold, deletion threshold, cutoff present, and cutoff absent.

9. The algorithm of claim 7, wherein said value of said at least one parameter is used to limit the number of said clinical data not yet investigated included in said best cost benefit consideration.

10. The algorithm of claim 1, further comprising creating output files consisting of abridged data cost quantity, abridged data cost procedure quantity, abridged parameter affected differential, and abridged global overview.

11. The algorithm of claim 10, wherein at least one of said output files is selected to limit the number of said clinical data not yet investigated included in said best cost benefit consideration.

12. The algorithm of claim 1, further comprising creating a plurality of complex clinical presentation models each comprising predetermined related diagnoses.

13. The algorithm of claim 12, wherein said related diagnoses are processed for concurrence with diagnoses in said differential diagnosis list having a probability P that reached said confirmation threshold parameter, said algorithm comprising the steps of:
a) matching each said diagnosis having a probability P that reached said confirmation threshold with at least one of said diagnoses comprised by at least one of said complex clinical presentation models;
b) selecting all said diagnoses comprised in said matched complex clinical presentation model and computing their probability P with a mini-max procedure; and
c) displaying those of said selected diagnoses that reach said confirmation threshold as concurrent diagnoses;
thereby precluding overlooking related diagnoses.

14. The algorithm of claim 1, further comprising creating a plurality of complex clinical presentation models each comprising a predetermined susceptible diagnosis with a susceptible clinical datum susceptible to be masked and predetermined masking diagnoses able to mask said susceptible clinical datum.

15. The algorithm of claim 14, wherein said masking diagnoses, said susceptible diagnosis, and said susceptible clinical datum are processed, said algorithm comprising the steps of:
a) matching each said susceptible diagnosis in said differential diagnosis reaching a predetermined cutoff present parameter and including said susceptible clinical datum absent with similar said susceptible diagnosis comprised by said complex clinical presentation model;
b) selecting all said masking diagnoses in said matched complex clinical presentation model and computing their probabilities P with a mini-max procedure;
c) displaying those of said selected masking diagnoses that reach said confirmation threshold parameter as concurrent diagnoses; and
d) computing again probability P of said susceptible diagnosis without considering said absent susceptible clinical datum if at least one of said masking diagnosis reaches said confirmation threshold parameter;
thereby precluding excessive reduction of probability P of said susceptible diagnosis by said masked clinical datum.

16. The algorithm of claim 1, further comprising creating input files for said diseases models, data present, data absent, complex presentation models, said clinical procedures, and said parameters.

17. The algorithm of claim 1, further comprising applying a mini-max procedure to said disease diagnoses in said differential diagnosis list, said mini-max procedure comprising:
a) creating predetermined clinical data pairs consisting of one of said clinical datum present in said patient and one clinical datum absent from said patient, said clinical datum absent being selected for sensitivity Sᵢ;
b) computing for each of said predetermined clinical data pairs a partial probability Pi in accordance with:
$$partial {P}_{i} = \frac{PP {value}_{i} ( 1 - {S}_{i} )}{\begin{matrix}PP {value}_{1} ( 1 - {S}_{1} ) + … PP {value}_{i} ( 1 - {S}_{i} ) + … \\ PP {value}_{n} ( 1 - {S}_{n} )\end{matrix}} ,$$
whereby said partial probabilities P₁ . . . Pₙ satisfy a normalization condition P₁+ . . . +Pₙ=1.

18. The algorithm of claim 17, wherein said mini-max procedure further comprises creating a mini-max table for each of said predetermined number of said disease diagnoses retained in said differential diagnosis list, whereby a first column of each said mini-max table comprises said PP value₁ through PP valueₙ for each said disease diagnosis and a first row of each said mini-max table comprises for each said predetermined data pair said sensitivity Sᵢ of said clinical datum absent.

19. The algorithm of claim 18, further comprising the steps of:
a) transferring each said partial probability Pᵢ into cells of said mini-max table where said PP valueᵢ for each said clinical datum present and said sensitivity Sᵢ for each said clinical datum absent converge; and
b) selecting from among said partial probabilities P₁ . . . Pₙ in said cells a determining partial probability P_{d} having the smallest value in its row and the greatest value in its column.

20. The algorithm of claim 19, wherein from each said mini-max table, said determining partial probability P_{d} is selected as a total probability Pₜ for said disease diagnosis for which said mini-max table was created.

21. The algorithm of claim 20, further comprising applying at least one evaluation function from the group consisting of said deletion threshold and said confirmation threshold to said total probability Pₜ for said disease diagnosis.

22. The algorithm of claim 21, further comprising determining a magnitude of change in said total probability Pₜ produced by a presence and by an absence of at least one clinical datum, thereby guiding a process of collection of said clinical data.

23. The algorithm of claim 1, wherein said clinical datum and said diagnoses employ an anti-aliasing scheme.

24. The algorithm of claim 13, wherein said anti-aliasing scheme comprises an alphanumeric identifier for synonymous diagnoses and synonymous clinical data.

25. The computer program of claim 1, further comprising applying a method enabling accurate processing of a limited number of diseases, without compromising accuracy of medical diagnosis process, creating models named other diseases and other diseases same representing all other diseases not included in the database with corresponding said disease models, thereby overcoming the condition that for accuracy all known diseases must exhaustively be included in the database with corresponding disease models.

26. An auxiliary medical diagnostic algorithm, said algorithm, named datum program, creating clinical datum lists, comprising for each clinical datum all the diagnoses able to manifest said clinical datum and displaying for each said diagnosis and said clinical datum the corresponding positive predicted value and sensitivity.
